(19) United States
(12) Patent Application Publication
(10) Pub. No.: US 2005/0201995 A1
(43) Pub. Date: Sep. 15, 2005

(54) Methods and compositions related to neuronal differentiation

(75) Inventor: Sadhan Majumder, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 11/051,668

(22) Filed: Feb. 4, 2005

Related U.S. Application Data
(60) Provisional application No. 60/582,586, filed on Feb. 5, 2004.

(51) Int. Cl. A61K 48/00; C12N 5/08

(52) U.S. Cl. 424/93.21; 424/93.7; 435/368

(57) ABSTRACT

Compositions and methods of the invention use a novel transactivator methodology for manipulation of the molecular mechanisms of cell determination for the production of a cell with a neuronal phentoype. A recombinant transcription factor or transactivator that binds the RE1 promoter element, REST-transactivator, was constructed by replacing the repressor domains of the transcriptional repressor REST with a transcriptional activation domain. The RE1 binding transactivator was designed to induce or manipulate the neuronal differentiation process.

### BACKGROUND OF THE INVENTION

This application claims priority to U.S. Provisional Patent Application Ser. No. 60/582,586, filed Feb. 5, 2004, which is incorporated herein by reference in its entirety.

The government owns rights in the present invention pursuant to grant number CA81255 from the National Cancer Institute.

### TECHNICAL FIELD

The present invention relates generally to the fields of molecular biology, neurobiology, and cell biology. More particularly, it concerns compositions and methods for differentiating a cell into a cell with a neuronal phenotype.

### DESCRIPTION OF RELATED ART

Neuroregeneration is a subject of intense study because of its potential to repair and restore damaged or diseased neurons. One approach to neuroregeneration is to manipulate various factors that control the ability of neuronal stem cells (NSCs) in the adult brain to regenerate (Galli et al., 2003; Goldman, 2003; Goldman and Nedergaard, 2002; Homer and Gage, 2002; Panchision and McKay, 2002). Another approach is to convert non-neural cells into the neuronal phenotype through differentiation or “transdifferentiation.” For this purpose, embryonic stem (ES) cells and bone marrow stem cells (BMSCs) have been used (Brivanlou, 2003; Frisen, 2002; Tsai et al., 2002). Such conversion of heterologous stem cells other than ES cells to neurons would have tremendous impact, overcoming most of the political and ethical concerns about using human ES cells for therapeutic purposes (Wurmser and Gage, 2002). However, it is not clear from these studies whether ES cell fusion rather than actual transdifferentiation is the mechanism for the formation of neurons from the BMSCs (Wummser and Gage, 2002), thus highlighting the importance of using a homogenous stem cell population as a starting material for transdifferentiation experiments.

C2C12 myoblasts represent an excellent, well-characterized mouse model for muscle differentiation (Walsh and Perlman, 1997). A homogeneous C2C12 myoblast cell culture, when cultured with high growth factors, proliferate as mononucleated progenitor cells. In contrast, when cultured with low growth factors, they enter the differentiation pathway, express muscle differentiation proteins, and fuse to form terminally differentiated, multinucleated myotubes. The molecular mechanisms responsible for this differentiation process have been studied extensively. Although less examined, C2C12 myoblasts are capable, under certain conditions, of differentiating into adipocyte-like cells (Teboul et al., 1995) or into an osteoblast lineage (Katagiri et al., 1994). The dedifferentiation of terminally differentiated C2C12 myotubes into proliferating mononucleated cells and their subsequent redifferentiation into cells expressing myogenic, chondrogenic, adipogenic, and osteogenic markers (McGann et al., 2001; Odelberg et al., 2000) further contribute to making C2C12 myoblasts an excellent yet simple system to study various principles of stem cell plasticity. Although these cells can be differentiated and dedifferentiated into various cell types, C2C12 myoblasts or dedifferentiated myotubes do not result in neuronal cells, suggesting the absence of positive factors, the presence of negative factors (including the intrinsic inability of these cells to become neurons), or both.

The differentiation of neural stem cells into neurons is generally believed to take place in four steps characterized by the expression and action of specific gene products (Immaneni et al., 2000; Lawinger et al., 2000). Thus, NSCs, which can multiply and make their own kind under one set of conditions and differentiate under other conditions, express p75 and nestin. The neuronal determination step is characterized by the action of the basic helix-loop-helix (bHLH) proteins, such as MASH, MATH, and NeuroD3/neurogenin. This is followed by the commitment step, in which genes such as neuroD1/2, Mytl, and neurofilament 150 are expressed. The terminal differentiation step is characterized by the expression of genes such as SCG10, sodium channel type II, synapsin, glutamate receptor, and acetylcholine receptor. It is mostly this group of terminal differentiation genes that are direct targets of the repressor element 1 (RE1)-silencing transcription factor (REST) otherwise known as neuron-restrictive silencer factor (NRSF) (REST/NRSF) (Chong et al., 1995; Schoenherr and Anderson, 1995).

REST/NRSF is expressed mostly in normeuronal cells and contains a DNA binding domain and two repressor domains. It blocks transcription of its target genes by binding to a specific consensus RE1 binding site/neuron-restrictive silencer element (RE1/NRSE) present in the genes' regulatory regions. REST/NRSF is downregulated for the induction and maintenance of the neuronal phenotype (Ballas et al., 2001) and overexpression of REST/NRSF in differentiating neurons disrupts neuronal gene expression and causes axon guidance errors (Paquette et al., 2000).

There remains a need for rapid and efficient methods and related compositions for differentiating cells into neuronal cells, in particular for the study and treatment of damaged or diseased neurons.

### SUMMARY OF THE INVENTION

Embodiments of the invention include expression vectors comprising a nucleic acid encoding a REST-transactivator, in certain aspects a REST-transactivator polypeptide. The REST-transactivator or REST-transactivator polypeptide may include a REST/NRSF element (RE1) binding domain and/or RE1 element binding domain and a transactivation domain. A REST-transactivator may be a small molecule, a peptide, a fusion protein or a combination thereof. In certain embodiments, an expression vector is a retrovirus. The transactivation domain may be choosen from various transactivator domains, both natural and synthetic, that are known to one of skill in the art. Transactivation domains include, but are not limited to a steroid/thyroid hormone nuclear receptor activation domain, a synthetic or chimeric activation domain, a polyglutamine activation domain, basic or acidic amino acid activation domain, a viral activation domain, VP16 activation domain, Tat activation domain, GAL4 activation domain, NF-kB activation domain, BP64 activation domain, TAF-1 activation domain, TAF-2 activation domain, TAU-1 activation domain, TAU-2 activation domain, a modified activation domain, or a fragment of any activation domain and/or modifications thereof. In cetain aspects the transactivator domain of a REST-transactivator may be all or part of a VP16 transactivation domain. The nucleic acid encoding the REST-transactivator will typically include a heterologous promoter. In certain aspects of the invention the heterologous promoter is a CMV promoter. In another aspect, the nucleic acid encoding the REST-transactivator may be operatively coupled to a regulatable promoter element, for example, a tetracycline regulated promoter element. The REST-transactivator may be comprised in a retrovirus. In certain aspects of the invention the retrovirus is a lentivirus. The retrovirus may be an Avian Leukosis Virus, a Bovine Leukemia Virus, a Murine Leukemia Virus, a Mink-Cell Focus-inducing Virus, a Murine Sarcoma Virus, a Reticuloendotheliosis virus or a Rous Sarcoma Virus.

In further embodiments, the invention includes a neuronal cell comprising a REST-transactivator. A REST-transactivator is a small molecule, peptide, and/or fusion protein that when introduced into a cell activates transcription of a gene having a RE1 repressor element. The RE1 element is typically situated in a genomic location so as to repress transcription from various genes, however, when a REST-transactivator is introduced it activates transcription, instead of repressing transcription, so when a transactivation moiety is localized to the RE1 element REST/NRSF repressed genes are transcribed. In certain aspects the REST-transactivator comprises a RE1 binding domain (recombinant, natural or synthetic) operably coupled to a transactivation domain (Recombinant, natural or synthetic). The transactivation domain may be choosen from the various transactivation domains, either recombinant, natural, or synthetic, that are known to one of skill in the art. In certain aspects the transactivation domain of a REST-transactivator may be all or part of a VP16 transactivation domain. The REST-transactivator may be encoded by an expression cassette. The expression cassette may be integrated into the genome of the cell or maintained episomally. The cell can be derived from a cell line, a primary cell, a stem cell or a progenitor cell. Stem cells or progenitor cells may be isolated from, but limited to muscle, cord blood, bone marrow, embryonic tissue or reproductive tissues. In partcular aspects of the invention a progenitor may be derived from a myoblast. In certain aspects of the invention, the neuronal cell is capable of forming spheroids, extensions of long axons, a synaptic network, and/or neuronal connections with other neuronal cells in vitro or in vivo.

Still further embodiments of the invention include methods for differentiating a cell into a neuronal cell comprising contacting the cell with an effective amount of a REST-transactivator or nucleic acid encoding a REST-transactivator. The REST-transactivator may be encoded by an expression cassette, as described above. The expression cassette may be comprised in a viral or non-viral expression vector. In certain aspects of the invention, the viral vector is an adenoviral or retroviral vector. The expression cassette may be integrated into the genome of the cell or maintained episomally in the cell. A REST-transactivator may be expressed ex vivo, in vitro or in vivo. The cell may be derived from a cell line, a primary cell, a progenitor cell and/or a stem cell. The cell may be isolated from muscle, cord blood, bone marrow, embryonic tissue or reproductive tissues. In certain aspects, the progenitor cell is a myoblast. The neuronal cell will typically be capable of forming spheroids, extensions of long axons, a synaptic network, and/or neuronal connections with a second neuronal cell in vitro.

Certain embodiments of the invention include methods of manufacturing a neuronal cell, as described herein, comprising the steps of: a) obtaining a cell; b) contacting the cell with an effective amount of a REST-transactivator, or a nucleic acid sequence encoding a REST-transactivator, to produce a neuronal cell; and c) culturing the neuronal cell. In certain aspects, the methods include deriving a cell from a first animal and administering the neuronal cell to a second animal. In a paritcular aspect of the invention the first and second animal are the same animal. The cell may be derived from a cell line, a primary cell, a progenitor cell and/or a stem cell. The cell may be isolated from, but not limited to muscle, cord blood, bone marrow, embryonic tissue or reproductive tissues. Progenitor cell may be isolated from muscle, bone marrow or neuronal tissue. In a particular aspect of the invention, the progenitor cell is a myoblast.

Yet further embodiments of the invention include methods of providing a therapeutic cell to animal comprising the steps of: a) obtaining a cell or neuronal cell in accordance with the compositions or methods described herein, and b) administering the cell or neuronal cell to the animal. Neuronal damage, loss or disease may be treated, abrogated, or reduced by administering to an animal in need thereof a cell comprising an effective amount of a REST-transactivator. Typically the cell will differentiate into a neuron or a neuronal-like cell before, during or after administration to a patient. The cell or neuronal cell may be administered by implantation, injection or perfusion. In certain aspects of the invention, the cell or neuronal cell is administered by, but not limited to intramuscular, intradural or intracranial injection or perfusion. In still further aspects the cells may be implanted in to the brain, spinal cord, muscle, or other neurologic or nerve containing tissue. In a particular aspect of the invention, the source of the cell or neuronal cell is a second animal. The first animal and the second animal may be the same animal. The cell or neuronal cell need not be differentiated prior to administration. The cell or neuronal cell may further differentiate once administered to the patient or animal. Aspects of the invention include providing a therapeutic cell to an animal, including but not limited to human, diagnosed with Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum defoimans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, ophthalmoplegia, brain trauma or injury, or spinal cord trauma or injury. In still further embodiments, REST-transactivator may be used in treating neoplasia, hyperplasia, tumors, neurologic tumors, and brain tumors. In certain embodiments administration of a REST-transactivator to a neoplastic or cancer may result in the cell terminal differentiating or under going apoptosis. Methods of treating cancer with REST-transactivators is contemplated. Treating may entail slowing growth, stopping growth, modulating growth or killing cancer cells.

Embodiments of the invention include, methods of abrogating or reducing neuronal damage or loss comprising administering to a neuronal cell or a neuronal progenitor cell an effective amount of a REST-transactivator. The neuronal damage or loss may be due to brain or spinal cord injury or trauma, or neurodegenerative disease or condition. In cetain aspects, the REST-transactivator is a REST-VP16 transactivator. The REST-transactivator may be encoded by an expression cassette and may be administered in vivo, in vitro, or ex vivo.

In still further embodiments of the invention includes one or more cells stably expressing a REST-transactivator produced by transforming a cell with an expression cassette encoding the REST-transactivator and selecting a cell that expresses the REST-transactivator. In certain aspects, the REST-transactivator is a REST-VP16 transactivator. The cell may be comprised in a pharmaceutically acceptable formulation and may be administered by injection, perfusion or implantation of the cell.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Neuronal differentiation of mammalian neural stem cells schematic.

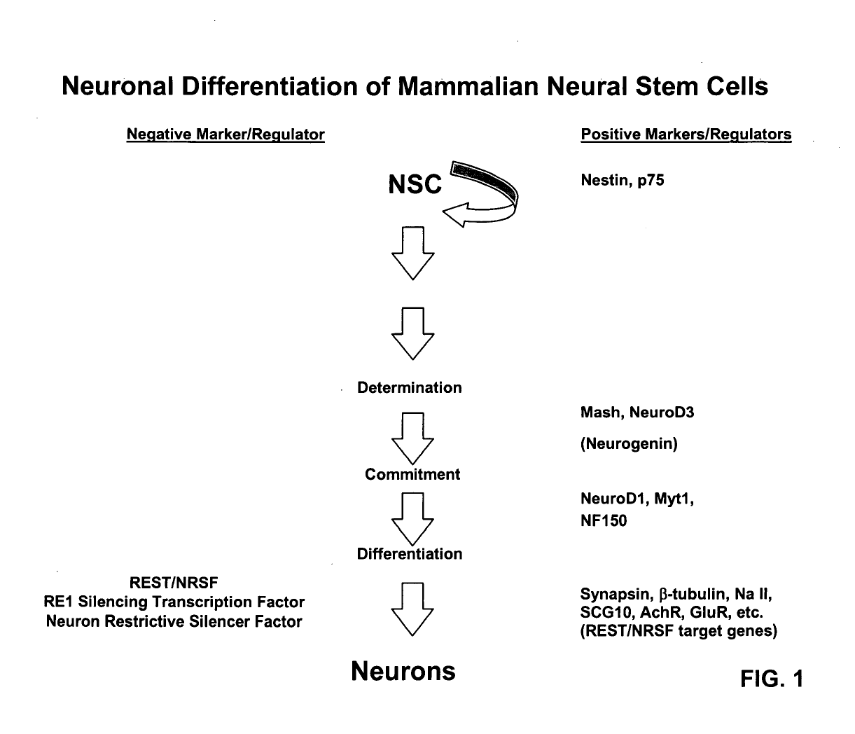

FIG. 2 Schematic of REST/NRSF transcriptional regulation.

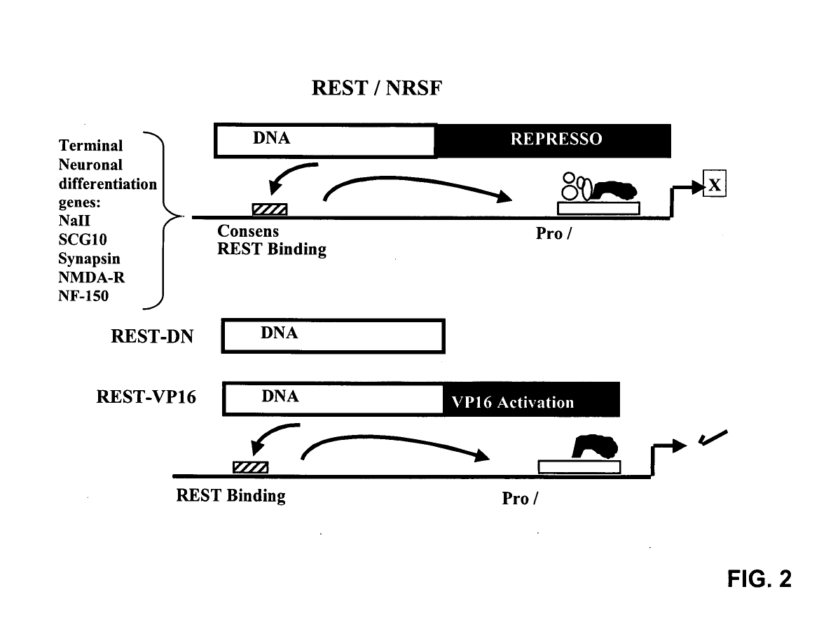

FIG. 3 Schematic of REST and REST transactivator proteins.

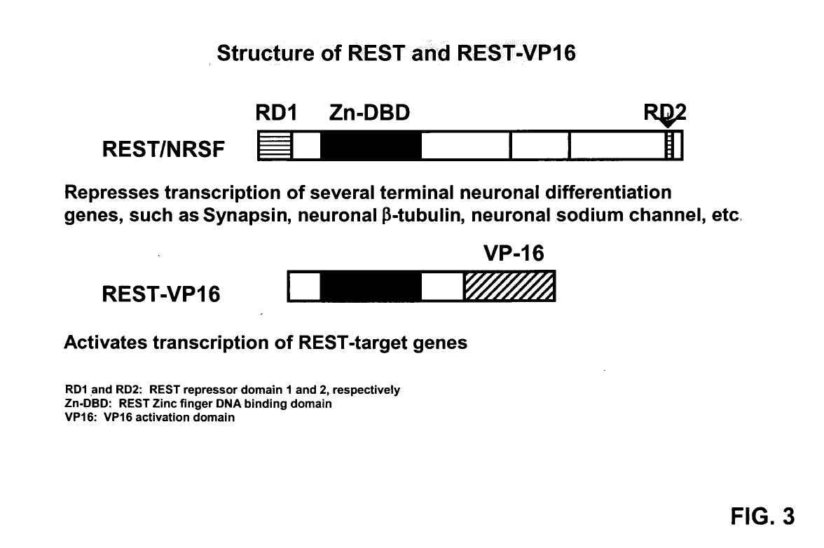

FIGS. 4A-4B. Myoblasts express REST/NRSF activity, which can be countered by R EST/NRSF-VP16. (FIG. 4A) C2C12 cells express endogenous REST/NRSF activity. Endogenous REST/NRSF activity was determined in C2C12 cells, PC12 cells (negative control) and HeLa cells (positive control) by transfection of a reporter plasmid containing the CAT reporter gene under the control of endogenous NaCh promoter with (+RE) or without (−RE) the REST/NRSF binding site. In these studies, cotransfection of an internal control plasmid, pCMV-O-gal, was used. The average CAT values, normalized to β-galactosidase activity, from triplicate studies are shown as a percent of NaCh (−RE) promoter activity. (FIG. 4B) The endogenous REST/NRSF activity in C2C12 cells can be countered by REST-VP16. Expression vector (Vector) and expression vector encoding REST/NRSF (pREST), Gal4-VP16 (pGal4-VP16) and REST-VP16 (pREST-VP16) were cotransfected with NaCh (+RE) or NaCh (−RE) reporter plasmids in C2C12 cells and the promoter activity was determined as in (FIG. 4A). The value for NaCh (−RE) in the presence of vector DNA was denoted as 100%.

FIGS. 5A-5B. Stable REST-VP16 expression in proliferating myoblasts causes activation of neuronal differentiation genes. (FIG. 5A) C2C12-REST-VP16 clonal cells (C2C12-RV) produced the REST-VP16 protein in the absence of doxycycline (−Dox) as assayed by western blotting using anti-VP16 antibodies. Such REST-VP16 production was not observed in the parental C2C12 cells or C2C12-RV cells in the presence of doxycycline (+Dox). When the same blot was probed with anti-synapsin antibody, expression of synapsin was observed in C2C12-RV(−Dox) cells but not in C2C12 cells. A low level of synapsin signal was also observed in C2C12-RV (+Dox) clones, presumably as a result of the leakiness of the doxycycline-regulated system. Expression of actin was measured as an internal control by using anti-actin antibodies. (FIG. 5B) The C2C12-RV (−Dox) cells producing the REST-VP16 protein can activate the basal TATA-box promoter through the RE1-binding sites. The cells were transfected with the reporter plasmid pTluc or pRE.Tluc along with an internal control plasmid, pβ-gal, and analyzed as described in the methods section. REST-VP16-dependent stimulation was calculated as pRE.Tluc expression/pTluc expression, and shown as relative luciferase activity.

FIGS. 6A-6D. Stable expression of functional REST-VP16 in myoblasts growing under muscle differentiation conditions blocks entry into muscle differentiation pathway and subsequent muscle formation and the MHC gene expression, causes the expression of neuronal differentiation genes and formation of neurite-like structures, but does not cause expression of glial differentiation genes. (FIG. 6A) C2C12, C2C12-RV (+Dox), and C2C12-RV (−Dox) cells were cultured in Dulbecco's Modified Eagle's Medium with 2% horse serum for 6 days (C2C12(D), C2C12-RV(D) (+Dox), and C2C12-RV(D) (−Dox), respectively) and examined by light microscopy, (FIG. 6B) western blot analysis, and (FIG. 6C) fluorescent microscopy. Anti-MAP2, anti-myosin heavy chain, and anti-synapsin antibodies were used. (FIG. 6D) Cell extracts prepared from C2C12 and C2C12-RV (−Dox) cells at various days in differentiation medium (day) were assayed by western blotting with anti-GFAP and anti-myogenin antibodies. Anti-actin was used as an internal control. Mouse brain extract was used as a positive control for GFAP expression.

FIGS. 7A-7D. Stable REST-VP16 expression in myoblasts enables them to survive mitotic inhibitors and exhibit neuronal phenotype. (FIG. 7A) C2C12-RV cells were cultured with and without Dox in differentiation medium without mitotic inhibitors for 6 days and then with mitotic inhibitors for another 14 days. These cells were then examined by immunofluorescence imaging with anti-MAP2 and anti-neurofilament (NF) antibodies. (FIG. 7B) Expression of secretogranin II in C2C12-RV(D) (−Dox) cells. Cell extracts from C2C12, C2C12(D), C2C12-RV(D) (+Dox) and C2C12-RV(D) (−Dox) cells were subjected to a western blot analysis with anti-secretogranin II antobody. Anti-actin was used as an internal control. (FIG. 7C) Expression of neuronal markers, such as neuronal β-tubulin, SCG10, synapsin (syn), BDNF, and Lrrnl in C2C12-RV(D) (−Dox) cells but not in C2C12 (D) (−Dox) cells as assayed by RT-PCR. Actin was used as an internal control. (FIG. 7D) Expression of synaptophysin and synaptotagmin in C2C12-RV(D) (−Dox) cells. Immunofluorescence analysis was performed with C2C12(D), C2C12-RV(D) (−Dox) cells using anti-synaptophysin (Syp) and anti-synaptotagmin (Syt) antibodies. Cell nuclei were stained with DAPI.

FIG. 8. Stable Expression of REST-VP16 in myoblasts growing under muscle differentiation conditions produced neurons with physiological properties. Differentiated C2C12 and C2C12-RV cells show depolarization-dependent calcium influx. C2C12, C2C12(D) or C2C12-RV(D) (−Dox) cells were loaded with 25 μM Fluo-3 AM for 60 min at room temperature. Fluo-3 AM solution was replaced with high KCl Ringer solution and image was taken at every 0.25 sec for a total of 5 seconds after depolarization. The results of three independent studies with C2C12-RV(D) (−Dox) cells are shown. Fluorescence intensity for experiment is shown at the bottom panel.

FIGS. 9A-9B. Stable Expression of REST-VP16 in myoblasts growing under muscle differentiation conditions produced neurons with physiological properties. (FIG. 9A) Uptake of FM1-43 by C2C12-RV(D) (−Dox) shows synaptic vesicle cycling with KCl depolarization. C2C12, C2C12(D) or C2C12-RV(D) (−Dox) cells were loaded with 15 μM FM1-43 with high KCl Ringer solution for 5 min at room temperature. Cells were washed with Ringer solution without a dye for 15 min to remove superficial FM1-43 on plasma membrane. The results of two independent experiments are shown. (FIG. 9B) Differentiated C2C12-RV cells show glutamate-induced calcium influx. C2C12 or C2C12-RV(D) (−Dox) cells were loaded with 25 μM Fluo-3 AM for 60 min at room temperature and the cells were treated as described in FIG. 8, except that 10 μM glutamate was added instead of KCl. The fluorescent signal returned to baseline upon wash.

FIG. 10. In vitro differentiated neurons produced by REST-VP16-expressing myoblasts incorporate into adult brain without forming tumors. C2C12 and C2C12-RV(D) cells were inoculated into the cerebellum of adult nude mice, sacrificed after 8 weeks and their brain tissues were formalin fixed, embedded in paraffin and used for light microscopic evaluation after staining by hematoxylin-eosine (H&E) or by immunohistochemical analysis with anti-VP16 antibody followed by anti-neuronal β-tubulin (Tuj1) antibody. The magnified view of C2C12-RV(D) cells show nuclear localization of VP16 signal and cytoplasmic localization of Tuj1 signal. Endogenous neuronal cells show cytoplasmic localization of Tuj 1 signal alone.

FIG. 11. Coculture of differentiated C2C12-REST-VP16 neuronal cells with differentiated C2C12 muscle fibers causes functional neuromuscular junction on muscle cells. C2C12 and C2C12-REST-VP16 cells were differentiated and assayed for acetylcholine receptor aggregation and detected by alexa647 conjugated-bungarotoxin staining. Arrows show muscle fiber boundary.

FIG. 12. REST-VP16 causes expression of neuronal differentiation marker in human mesenchymal stem cells. Human mesenchymal stem cells were transfected with pcDNA 3.1 vector DNA or pcDNA 3.1 vector DNA containing REST-VP16, and subjected to immunocytochemistry to detect the production of neuronal differentiation marker, neuronal β-tubulin, using Tuj-1 antibody (green). The cell-nuclei were stained with DAPI.

FIGS. 13A-13B. Expression of functional REST-VP16 in stable neural stem cell (NSC) clones. (FIG. 13A) C17.2 cells (NSC) and stable C17.2 clones encoding the vector (NSC-V), or REST-VP16 (NSC-RV) were grown in growth medium (Materials and Methods) with and without doxycycline (Dox) for 2 days, and the cell extracts (20 μg) were analyzed by western blotting with anti-VP16 and anti-α-tubulin antibodies. (FIG. 13B) The NSC-RVS (−Dox) cells producing the REST-VP16 protein can activate the basal TATA-box promoter through the RE1-binding sites. To determine whether the REST-VP16 protein was transcriptionally active, the inventors constructed synthetic promoters containing a luciferase reporter gene under the control of a TATA box alone (pT.luc) or a TATA box plus two copies of the wild-type RE1/neuron-restrictive silencing factor (NRSE) sequence (pRE.T.luc). The cells were grown without doxycycline and were transfected with the reporter plasmid pT.luc or pRE.T.luc along with an internal control plasmid (pp-gal), and analyzed as described herein. Luciferase expression is shown for pT.luc and p.RE.T.luc.

FIGS. 14A-14B. REST-VP16 causes neuronal differentiation of NSCs in vitro. (FIG. 14A) NSC-RVs show neuronal β-tubulin expression. NSCs, NSC-Vs, or NSC-RVs were plated on polylysine-laminin-coated dishes and were grown in growth medium with and without doxycycline. After the indicated times in culture, the cellular nuclei were stained with Hoechst dye, and the cells were then analyzed by immunofluorescence microscopy (60× magnification) with anti-Tuj1 antibodies to measure expression of neuronal β-tubulin. The magnified views of the 16-day images (−Dox) show the cellular morphology. (FIG. 14B) NSC-RVs show synaptotagmin I expression. Cell extracts (20 μg) from NSC-Vs and NSC-RVs cultured for 16 days as described in FIG. 14A were subjected to western blot analysis using anti-VP16 (VP16), anti-synaptotagmin I (syt), and anti-actin antibodies. Mouse brain extract was used as a positive control for synaptotagmin I expression.

FIG. 15. REST-VP16 caused expression of neuronal β-tubulin in NSCs. NSCs, NSC-Vs, or NSC-RVs were grown with retinoic acid and mitotic inhibitors in the presence or absence of doxycycline (Dox), and the cellular nuclei were stained with Hoechst dye. The cells were then analyzed by immunofluorescence microscopy (60× magnification) for neuronal β-tubulin (Tuj1).

FIG. 16. REST-VP16 caused expression of MAP2 in NSCs. NSCs, NSC-Vs, or NSC-RVs were grown with retinoic acid and mitotic inhibitors in the presence or absence of doxycycline (Dox), and the cellular nuclei were stained with Hoechst dye. The cells were then analyzed by immunofluorescence microscopy (60× magnification) for MAP2.

FIG. 17. NSC-RVs show depolariiation-dependent calcium influx. NSCs, NSC-Vs, or NSC-RVs (−Dox) from the cells shown in FIG. 16 were loaded with 25 μM Fluo-3 AM for 60 min at room temperature. The Fluo-3 AM solution was replaced with high-KCl Ringer solution, and an image was taken every 0.25 s for a total of 12 s after depolarization. Fluorescence intensity for experiments is shown in the bottom panel. The scale bar represents 60 μm.

FIG. 18. NSC-RVs show glutamate-induced calcium influx. NSCs, NSC-Vs, or NSC-RVs (−Dox) cells were loaded with 25 μM Fluo-3 AM for 60 min at room temperature, and the cells were treated as described in FIG. 17, except that 10 μM glutamate was added instead of KCl, and an image was taken every 0.25 s for a total of 12 s. Fluorescence intensity for the study is shown in the bottom panel. The scale bar represents 60 μm.

FIGS. 19A-19C. Histologic analysis of normal human brain tissue sections (FIG. 19A) and medulloblastoma tumor specimens (FIG. 19B). Paraffin-embedded tissue sections were processed, stained with H&E, and analyzed. Primary medulloblastoma specimens overexpress REST/NRSF (FIG. 19C). Immunohistochemical analysis was performed using paraffin-embedded HeLa cytospin preparation, normal cerebellum tissue, and medulloblastoma primary tumor specimens with preimmune serum and an anti-REST antibody. HeLa cells have highlevels of REST/NRSF and served here as a positive control. Magnified versions of medulloblastoma specimens in the presence of the preimmune serum and anti-REST antibody are also shown to exhibit nuclear localization of REST/NRSF.

FIG. 20. REST-VP16 blocks the tumorigenic potential of medulloblastoma cells at intracranial locations in nude mice. Medulloblastoma cell lines (D283 or Daoy) infected with Ad or Ad.REST-VP16 were inoculated intracranially into nude mice using a guidescrew system, and their brains were examined histologically after 5 weeks, as described in Materials and Methods. The needle mark produced during the inoculation of cells can be seen in the REST-VP16 specimens.

FIG. 21. Adenovirus-mediated delivery of REST-VP16 inhibits growth of medulloblastoma cells at intracranial locations in nude mice. Medulloblastoma cell lines (D283 or Daoy) were inoculated intracranially into nude mice using a guide-screw system and after a two-week period, the implanted cells were injected with Ad.GFP or Ad.REST-VP16 and their brains were examined by MRI (left panel; T1, T1+Gd, and T2) and by histological methods (right panel; H&E). The example shows the largest tumors observed for each group (arrow). Spherical dark MRI susceptibility artifacts due to the presence of contaminating metal from the guidescrews in the shape of a circle are present in some of the images.

FIG. 22. Adenovirus-mediated delivery of REST-VP16 causes expression of synaptophysin in medulloblastoma cells at intracranial locations in nude mice. Preformed medulloblastoma tumors injected with either Ad.GFP or Ad.REST-VP16, as described in FIG. 21, were sectioned and subjected to immunohistochemical analysis using anti-synaptophysin antiobody.

FIG. 23. Adenovirus-mediated delivery of REST-VP16 causes apoptosis of medulloblastoma cells at intracranial locations in nude mice. Preformed medulloblastoma tumors injected with either Ad.GFP or Ad.REST-VP16, as described in FIG. 21 were sectioned and subjected to an in situ apoptosis detection assay. The brown nuclear signal indicates apoptosis.

FIG. 24. A role of REST/NRSF in medulloblastoma. REST-VP16 can counter REST/NRSF's function in human medulloblastoma cells and block the cells' tumorigenic potential.

### DESCRIPTION OF ILLUSTRATIVE EMBODIMENTS

Regeneration of neurons is being studied as a way to repair and restore damaged or diseased neurons. Embodiments of the invention address various deficiencies in the compositions and methods related to generation or regeneration of neuorns, such as production, differentiation and utilization of cells with a neuronal phenotype. Compositions and methods of the invention use a transactivator methodology for manipulation of the molecular mechanisms of cell determination for the production of a cell with a neuronal phentoype. An exemplary recombinant transcription factor or transactivator, REST-VP16, was constructed by replacing the repressor domains of the transcriptional repressor REST with the activation domain of the herpes simplex virus protein VP16. The RE1 binding transactivator was designed to induce or manipulate the neuronal differentiation process. Because REST-VP16 still contains the DNA binding domain of REST/NRSF the REST-VP16 protein operates through the RE1/NRSE, competes with endogenous REST/NRSF for DNA binding, and activates the normally repressed REST/NRSF target genes (Immaneni et al., 2000; Lawinger et al., 2000). For an exemplary description of the RE1/NRSE see Wood et al., 2003, Bai et al., 2003, Garcia-Sanchez et al., 2003, Mbikay et al., 2002, Mieda et al., 1997, Eggen and Mandel, 1997, or Wood et al., 1996, each of which is incorporated herein by reference. The inventors contemplate that a polypeptide domain that binds the RE1/NRSE element may be fused with a variety of transcriptional activation domains known in the art, thus the invention is not limited to a REST-VP16 fusion protein and may include various other transactivators that are exemplified by the REST-VP16 polypeptide. In addition, a variety of modified REST/NRSF DNA binding domains are contemplated as long as a minimal binding affinity or transcriptional activation activity of the fusion protein is maintained. In still further embodiments, it is contemplated that artificial transactivators, wherein the DNA binding domain is replaced by a minimal DNA binding peptide or petidomimetic compound and the transactivation domain is a minimal peptide or peptidomimetic transactivation domain, which may be used in the methods described herein.

The studies of Immaneni et al. and Lawinger et al. have demonstrated the ability of a transiently expressed REST-VP16 transactivator to activate transcription of various genes typically repressed by endogenous REST/NRSF, i.e., REST-VP16 counters REST/NRSF activity. However, neuronal differentiation was not achieved by these methods. Embodiments of the present invention include compositions and methods for the transcriptional activation of REST/NRSF target genes by a RE1/NRSE binding/transactivator fusion protein, e.g., REST-VP16, for the conversion, induction, or promotion of a cell to a neuronal phenotype. Certain embodiments include contacting a cell with REST-VP16, or a polypeptide with similar characteristics, by administration of a polypeptide or by introduction of an expression cassette that encodes such a polypeptide to effect a neuronal cell phenotype.

Neuronal differentiation is typically defined as the acquisition of specific biochemical, physiological, and morphological properties of a neuron, i.e., acquiring and typically maintaining a neuronal phenotype. In various embodiments of the invention the molecular mechanisms of neuronal differentiation may be modulated or manipulated by a fusion protein or an artificial transactivator that binds to repressor elements controlling and/or regulating expression, e.g., transactivation, of various genes required for the expression of a neuronal phenotype.

Various cells can be induced to differentiate into a variety of cell types by biochemical manipulation of their environment. Mammalian neuronal stem cells have been isolated that can be converted over time into neurons and other cell types by maintaining the cells under various growth conditions. The neuronal differentiation pathways were previously thought to be regulated primarily through positive regulators such as growth factors and the like. Several genes encoding such regulators and their cellular interactions were identified through analysis of mammalian and non-mammalian embryogenesis, regeneration, repair and disease (Mandel and McKinnon, 1993; St-Jacques and McMahon, 1996; Rowitch et al., 1997; Brunet et al., 1998; Mariani and Harland, 1998; McMahon et al., 1998). A Variety of cells, such as cell lines, primary cells, progenitor cells, stem cells, myoblasts, and neuronal stem cells, can be manipulated by the methods described herein to produce a neuronal cell using methods that accelerate the formation of and that differentiate diverse cell types into neuronal cells.

An exemplary cell source, pluripotent stem cells, can be derived from various somatic and embryonic sources. In the mouse, one type of pluripotent stem cell can be isolated from cells of the inner cell mass of a pre-implantation embryo and are termed embryonic stem (ES) cells (Evans and Kaufman, 1981). A second type of pluripotent stem cell can be isolated from primordial germ cells (PGCs) located in the genital ridges of post coitum embryos (day 8.5 post coitum mouse embryos). Post coitum cells have been referred to as embryonic germ cell (EG) (Matsui et al., 1991; Resnick et al., 1992; and U.S. Pat. No. 5,453,357). Other stem cells include, but are not limited to bone marrow stem cells, mesenchymal stem cells and the like.

I. REST/NRSF and REST/NRSF Related Polypeptides

Schoenherr and Anderson (1995) reported the cloning of a transcription factor that binds the neuron-restrictive silencer element (NRSE) in the 5-prime regulatory region of the neuron specific gene SCG10. The transcription factor was referred to as the neuron-restrictive silencer factor (NRSF). The NRSF cDNA was cloned from a HeLa cell library and the expressed protein was shown to bind the NRSE DNA sequence. Expression of NRSF mRNA was detected in most normeuronal progenitor cells but was absent in differentiated neurons. Schoenherr and Anderson (1995) proposed that NRSF may function as a master negative-regulator of neurogenesis. Chong et al. (1995) also discovered this protein and named it REST. The International Radiation Hybrid Mapping Consortium mapped the REST gene to chromosome 4. Hence the repressor may be referred to as REST, NRSF or REST/NRSF.

Thiel et al. (1998) determined that REST/NRSF contains 2 repressor domains, one located at the N-terminus and the other at the C-terminus, and an N-terminal zinc finger cluster which functions as the DNA-binding domain for neuronal genes. Subsequently, Palm et al. (1999) identified several REST/NRSF variants that arise from alternative splicing of an exon that they designated exon N. The splice variants produce insertions that generate in-frame stop codons and encode truncated proteins with an N-terminal repressor domain and weakened DNA-binding activity.

Thiel et al. (1998) noted that the REST/NRSF binding sites of several neuron-specific genes, such as those encoding synapsin I, SCG10, A1-glycine receptor, the B2 subunit of the nicotinic acetylcholine receptor, and the M4-subunit of the muscarinic acetylcholine receptor, are found at various positions within the sequence. By transfecting these sequences in reporter constructs together with REST/NRSF, Thiel et al. (1998) found that REST/NRSF blocks transcription of a gene irrespective of whether the NRSE is located upstream or downstream of the open reading frame in either orientation and in both a distance- and a gene-independent manner.

Using indexing-based differential display PCR on neuronal precursor cells to study gene expression in Down syndrome, Bahn et al. (2002) found that genes regulated by the REST/NRSF transcription factor were selectively repressed. One of these genes, SCG10, was almost undetectable. In cell culture, the Down syndrome cells showed a reduction of neurogenesis, as well as decreased neurite length and abnormal changes in neuron morphology. In addition, Zuccato et al. (2003) have shown that REST/NRSF interacts with the Huntingtin protein and modulates the transcription of RE1/NRSE containing neuronal genes. Thus, the transactivation of REST/NRSF may have a therapeutic effect in patients with Hunington's disease.

A. RE1-Binding Transactivator Polypeptides

Embodiments of the invention include transactivators and/or transactivator polypeptides that bind to repressor element 1 (RE1 elements) present in the transcription regulatory regions of various genes. Examples of the RE1 element can be found in Wood et al. (1996). The RE1 binding transactivators include a RE1 element binding domain, preferably a REST/NRSF DNA binding domain or variant thereof, fused to a transactivation domain. Transactivation domains include, but are not limited to a steroid/thyroid hormone nuclear receptor activation domain, a synthetic or chimeric activation domain, a polyglutamine activation domain, basic or acidic amino acid activation domain, a viral activation domain, VP16 activation domain, Tat activation domain, GAL4 activation domain, NF-kB activation domain, BP64 activation domain, TAF-1 activation domain, TAF-2 activation domain, TAU-1 activation domain, TAU-2 activation domain, a modified activation domain, or a fragment of any activation domain and/or modifications thereof. In preferred embodiments the transactivation domain is a VP16 transactivation domain. However, the inventors contemplate that other transactivation domains known in the art may used, such as a transactivation domain from any naturally occurring or synthetic transcriptional activator. For a general discussion of transcription factors and particularly activation domains see Locker (2001).

In certain embodiments, the present invention concerns compositions comprising a REST-VP16 fusion protein. One example of a REST-VP16 fusion polypeptide is presented as SEQ ID NO:2 or SEQ ID NO:4, as encoded by the exemplary nucleic acid of SEQ ID NO:1 or SEQ ID NO:3, respectively. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 5 to about 100 amino acids. All the “proteinaceous” terms described above may be used interchangeably herein.

In certain embodiments the size of the at least one proteinaceous molecule may comprise, but is not limited to about 50, about 60, about 70, about 80, about 90, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues, and any range derivable therebetween.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino acid molecule sequences comprising at least one of the 20 common amino acids in naturally synthesized proteins, or at least one modified or unusual amino acid, as known in the art.

In certain embodiments the proteinaceous composition comprises at least one protein, polypeptide or peptide. In further embodiments the proteinaceous composition comprises a biocompatible protein, polypeptide, or peptide. As used herein, the term “biocompatible” refers to a substance which produces no significant untoward effects when applied to, or administered to, a given cell, tissue, or organism according to the methods and amounts described herein or known to those of skill in the art. In preferred embodiments, biocompatible protein, polypeptide or peptide containing compositions will generally be natural proteins, mammalian proteins, fusion proteins, peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes, such as REST/NRSF (genbank accession numbers U22314 and NM_{—}005612) and VP16 activation domain (e.g., nucleotide 451-837 of genbank accession number AY136632 or amino acids 150-278 of genbank accession number AAN86074), have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or as would be know to those of ordinary skill in the art.

In certain embodiments, a proteinaceous compound may be purified. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In still further embodiments, a proteinaceous composition may be comprised in therapeutic compositions. In particular embodiments, humans are desired patients for treatment with proteins or other active ingredients of the present invention. The dosage regimen of a protein-containing pharmaceutical composition to be used in neuronal regeneration or differentiation will be determined by the attending physician. The physician may consider various factors which modify the action of the pharmaceutical composition, e.g., amount of tissue weight or number of cells in which a neuronal phentoype is desired, the site of damage or deficiency, the condition of the damaged tissue, type of damaged tissue (e.g., brain or spinal cord), the patient's age, time of administration and other clinical factors. The dosage may vary with the type of target tissue or cell, and with inclusion of other proteins in the pharmaceutical composition. For example, the addition of known growth factors to the final composition, may also effect the dosage. Effective dosages of the composition can be determined by the attending physician and may include dosages in the range of about 0.1. 1, 2, 3, 4, 5, 6, 7, 8, 9, 10, 20, 30, 40, or 50 mg/kg to about 40, 50, 60, 70, 80, 90, or 100 mg/kg of patient body weight or any range between or within. For example about 0.1 mg/kg to about 50 mg/kg of patient body weight, or about 10 mg/kg to about 50 mg/kg of patient body weight.

1. Fusion Proteins

In certain embodiments, a polypeptide may be a fusion protein that comprises multiple polypeptides, or peptide or peptidomimetic domains. A fusion partner may, for example, assist in providing transcriptional activation functions to a RE1 binding domain or polypeptide. Certain preferred fusion partners are fusion partners that enhance the expression of REST/NRSF regulated genes. Fusion proteins may also include affinity tags, which facilitate purification or identification of the protein or cellular uptake sequences that enhance the transport of a polypeptide across a cellular membrane.

Fusion proteins may generally be prepared using standard techniques, including genetic fusion or chemical conjugation. A fusion protein can be expressed as a recombinant protein, allowing the production of increased levels in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase or frame. This permits translation into a single fusion protein that retains all or part of the biological activity of both component polypeptides.

A peptide or non-peptide linker may be employed to separate the first and second polypeptide components by a distance sufficient to ensure that each polypeptide folds into its secondary and tertiary structures. Such a peptide linker sequence may be incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional domains on the first and second polypeptides; and (3) the lack of hydrophobic or charged residues that might react with the polypeptide functional domains. Preferred peptide linker sequences contain Gly, Asn, and/or Ser residues. Other near neutral amino acids, such as Thr and Ala may also be used in the linker sequence. Amino acid sequences which may be usefully employed as linkers include those disclosed in Maratea et al., 1985; Murphy et al., 1986; U.S. Pat. Nos. 4,935,233 and 4,751,180, each incorporated herein by reference. The linker sequence may generally be from 1 to about 50 amino acids in length, including but not limited to the various lengths therebetween. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are typically located only 5′ to the DNA sequence encoding the first polypeptides. Similarly, stop codons required to end translation and transcription termination signals are present 3′ to the DNA sequence encoding the second polypeptide.

2. Polypeptide Variants

Conservative amino acid substitutions generally are based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as equivalent.

The importance of the hydropathic amino acid index in conferring interactive biological function on a protein is generally understood in the art (Kyte and Doolittle, 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within +1 are particularly preferred, and those within +0.5 are even more particularly preferred.

The use of hydrophilicity values are described, for example in U.S. Pat. No. 4,554,101. In making changes based upon similar hydrophilicity values, the substitution of amino acids whose hydrophilicity values are within ±2 is preferred, those which are within +1 are particularly preferred, and those within ±0.5 are even more particularly preferred.

II. REST-Transactivator Nucleic Acids

Embodiments of the invention include nucleic acids encoding fusion proteins comprising a RE-1 DNA binding domain fused with a transactivation domain. Certain aspects of the invention may include nucleic acids encoding REST-transactivators and in ceratin aspects REST-VP16 transactivators. Certain embodiments of the present invention concern all or part of the nucleic acid of SEQ ID NO:1 or SEQ ID NO:3. In certain aspects, both wild-type and mutant versions of REST-transactivator sequences are employed. In other aspects, a nucleic acid comprises a nucleic acid segment of SEQ ID NO:1 or SEQ ID NO:3, or a biologically functional equivalent thereof.

In certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this functional term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, and/or mutants.

A nucleic acid may encompass a double-stranded molecule or a triple-stranded molecule that comprises one or more complementary strand(s) or “complement(s)” of a particular sequence. As used herein, a single stranded nucleic acid may be denoted by the prefix “ss,” a double stranded nucleic acid by the prefix “ds,” and a triple stranded nucleic acid by the prefix “ts.”

“Isolated substantially away from other coding sequences” means that the gene of interest forms a part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

In certain aspects, the present invention concerns a nucleic acid encoding a REST-transactivator that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

A nucleic acid of the invention may be purified on polyacrylamide gels, cesium chloride centrifugation gradients, or by any other means known to one of ordinary skill in the art (see for example, Sambrook et al., 2001, incorporated herein by reference).

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used and/or coupled together to form a larger coding region. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

B. Nucleic Acid Segments

In certain embodiments, the nucleic acid is a nucleic acid segment. As used herein, the term “nucleic acid segment,” are smaller fragments of a nucleic acid, such as the non-limiting example of SEQ ID NO:1 or SEQ ID NO:3. Thus, a “nucleic acid segment” may comprise any part of a gene sequence, of from about 8 nucleotides to the full length of a nucleic acid, e.g., SEQ ID NO:1 or SEQ ID NO:3.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
   where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm would be applied to each of SEQ ID NO:1 or SEQ ID NO:3. As used herein, a “probe” generally refers to a nucleic acid used in a detection method or composition. As used herein, a “primer” generally refers to a nucleic acid used in an extension or amplification method or composition.

In a non-limiting example, one or more nucleic acid constructs may be prepared that include a contiguous stretch of nucleotides identical to or complementary to SEQ ID NO:1 or SEQ ID NO:3. A nucleic acid construct may be about 50, about 60, about 70, about 80, about 90, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 14, about 15, about 16, about 17, about 18, about 19, about, 20, about 21, about 22, about 23, about 24, about 25, about 26, about 27, about 28, about 29, about 30, about 35, about 40, about 125, about 150, about 175, about 750, about 1,500, about 12,000, about 55,000, about 175,000, about 275,00, about 1,250,000 or more bases.

C. Nucleic Acid Complements

The present invention also encompasses a nucleic acid that is complementary to a REST-transactivator, including, but not limted to SEQ ID NO:1 or SEQ ID NO:3. A nucleic acid “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule of a REST-transactivator, e.g., SEQ ID NO:1 or SEQ ID NO:3, during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

D. Genetic Degeneracy

The term “functionally equivalent codon” is used herein to refer to codons that encode the same amino acid, such as the six codons for arginine and serine, and also refers to codons that encode biologically equivalent amino acids. For optimization of expression in human cells codons may be used that are preferred codons in human cells, for example the most preferred codon for alanine is thus “GCC”, and the least is “GCG.”. Codon usage for various organisms and organelles can be found at the website www.kazusa.orjp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences described or disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:1 or SEQ ID NO:3 will be nucleic acid sequences that are “essentially as set forth in SEQ ID NO:1 or SEQ ID NO:3, respectively.”

III. Vectors and Expression Constructs

The term “expression vector” is used to refer to a carrier nucleic acid molecule into which a nucleic acid sequence can be inserted for introduction into a cell where it can be replicated and/or expressed. A nucleic acid sequence can be “exogenous,” which means that it is foreign to the cell into which the expression vector is being introduced or that the sequence is homologous to a sequence in the cell but in a position within the host cell in which the sequence is ordinarily not found. Expression vectors include nucleic acids sucha as plasmids, cosmids, viruses (bacteriophage, animal viruses, and plant viruses), and artificial chromosomes (e.g., YACs). One of skill in the art would be well equipped to construct an expression vector through standard recombinant techniques (see, for example, Sambrook et al., 2001 and Ausubel et al., 1996, both incorporated herein by reference). In certain embodiments of the invention the expression vector or parts thereof will be maintained in a cell for a sufficient period time to induce, produce, or result in neuronal differentiation of the cell that contains all or part of the expression vector. In certain embodiments the REST-transactivator is stably expressed in a host cell. Stable transformation of a cell with an expression vector is well known in the art, examples of which can be found in Sambrook et al. 2001.

The term “expression cassette” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression cassettes can contain a variety of “control sequences,” which refer to nucleic acid sequences necessary for the transcription and possibly translation of an operable linked coding sequence in a particular host cell. In addition to control sequences that govern transcription and translation, expression vectors and expression cassettes may contain nucleic acid sequences that serve other functions as well and are described herein.

In order to express a REST-transactivator polypeptide it is necessary to provide a REST-transactivator gene in an expression vector or expression cassette. The appropriate nucleic acid can be inserted into an expression vector or cassette by standard subcloning techniques. For example, an E. coli or baculovirus expression vector is used to produce recombinant polypeptide in vitro. The manipulation of these expression vectors is well known in the art. In one embodiment, the protein is expressed as a fusion protein allowing rapid affinity purification of the protein. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these fusion systems produce recombinant protein bearing only a small number of additional amino acids, which are unlikely to affect the functional capacity of the recombinant protein. Other fusion systems produce proteins where it is desirable to excise the fusion partner from the desired protein. In another embodiment, the fusion partner is linked to the recombinant protein by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

Recombinant bacterial cells, for example E. coli, are grown in any of a number of suitable media, for example LB, and the expression of the recombinant polypeptide induced by adding IPTG to the media or switching incubation to a higher temperature. After culturing the bacteria, the cells are collected by centrifugation and washed to remove residual media. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are enriched by incorporation of sugars such as sucrose into the buffer and centrifugation at a selective speed.

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.).

There also are a variety of eukaryotic vectors that provide a suitable vehicle in which recombinant polypeptide can be produced. HSV has been used in tissue culture to express a large number of exogenous genes as well as for high level expression of its endogenous genes (Herz and Roizman (1983)). The lacZ gene also has been expressed under a variety of HSV promoters.

In preferred embodiments, the nucleic acid is under transcriptional control of a promoter. A “promoter” refers to a DNA sequence recognized by the synthetic machinery of the cell required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Promoters also include those promoters that can be regulated by, but not limited to, temperature, small molecules, cellular environment or by other regulatory means known in the art, see Miller and Whelan, 1997; Haviv and Curiel, 2001 and similar publications. One preferred embodiment is the tetracycline controlled promoter (for example see Haberman and McCown, 2002). In various other embodiments, the human cytomegalovirus (CMV) immediate early gene promoter, the SV40 early promoter and the Rous sarcoma virus long terminal repeat can be used to obtain high-level expression of transgenes. The use of other viral, mammalian, or bacterial promoters which are well-known in the art to achieve expression of a transgene is contemplated as well, provided that the levels of expression are sufficient for a given purpose. Table 1 lists several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more element that directs initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct. Promoters may include, but are not limited to Immunoglobulin Heavy Chain, Imunoglobulin Light Chain, T-Cell Receptor, HLA DQ α and DQ β, β-Interferon, Interleukin-2, Interleukin-2 Receptor, MHC Class II 5, MHC Class II HLA-DRα, β-Actin, Muscle Creatine Kinase, Prealbumin (Transthyretin), Elastase I, Metallothionein, Collagenase, Albumin Gene, α-Fetoprotein, τ-Globin, β-Globin, c-fos, c-HA-ras, Insulin, Neural Cell Adhesion Molecule (NCAM), α_{1-Antitrypsin}, H₂B (TH2B) Histone, Mouse or Type I Collagen, Glucose-Regulated Proteins (GRP94 and GRP78), Rat Growth Hormone, Human Serum Amyloid A (SAA), Troponin I (TN I), Platelet-Derived Growth Factor, Duchenne Muscular Dystrophy, SV40, Polyoma, Retroviruses, Papilloma Virus, Hepatitis B Virus, Human Immunodeficiency Virus, Cytomegalovirus, Gibbon Ape Leukemia Virus promoters. Use of the baculovirus system will involve high level expression from the powerful polyhedron promoter.

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

TABLE 1

| Element | Inducer |
|---|---|
| MT II | Phorbol Ester (TPA) |
| | Heavy metals |
| MMTV (mouse mammary tumor | Glucocorticoids |
| virus) | |
| β-Interferon | Poly(rI)X |
| | Poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| □-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone □ | Thyroid Hormone |
| Gene | |

In various embodiments of the invention, the expression vector or expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

A. Cell Transformation

Various methods of gene transfer for transient transfection, stable transfection, and cotransfection include a variety of expression vectors. Expression vectors include, but are not limited to viral, plasmid, linear expression elements, and circular expression elements. Cells may be transfected by electroporation (Gene Pulser, BIO RAD, Hercules, Calif.) with a recombinant plasmid or other expression vector that may contain a selectable marker. Cells can be cultured in selective media, for example a media containing G418 (Geneticin; GIBCO). The surviving cells can be used for further characterization or in therapeutic methods or processes. Success of stable transformation can be confirmed by Northern blot analysis as well as various phenotypic assays.

To achieve stable expression of a REST-transactivator, a transfected expression vector or expression cassette can be integrated into the host cell genome. Alternatively, the transfected expression vector or expression cassette can be maintained as a stable episome. For long-term production of recombinant proteins, stable expression is generally preferred. For example, cell lines which stably express a polynucleotide of interest may be transformed using expression vectors or expression cassettes which may contain viral origins of replication and/or endogenous expression elements and/or a selectable marker gene on the same or on a separate vector or cassette. Following the introduction of the expression vector, cells may be allowed to grow for a number of days, typically 1 to 2 days, in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., 1977) and adenine phosphoribosyltransferase (Lowy et al., 1990)) genes which can be employed, in tk⁻ or aprt⁻ cells, respectively. Also, antimetabolite or antibiotic resistance can be used as the basis for selection; for example, dhfr which confers resistance to methotrexate (Wigler et al., 1980); and npt, which confers resistance to the aminoglycosides, neomycin and G-418 (Colbere-Garapin et al., 1981). Additional selectable genes have been described, for example, trpB, which allows cells to utilize indole in place of tryptophan, or hisD, which allows cells to utilize histinol in place of histidine (Hartman and Mulligan. 1988). Visible markers may also be used, such as anthocyanins, beta-glucuronidase and its substrate GUS, and luciferase and its substrate luciferin, green fluorescent protein (GFP) and the like being widely used not only to identify transformants, but also to quantify the amount of transient or stable protein expression attributable to a specific vector system (Rhodes et al., 1995).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter, typically by including an IRES between the genes to be translated. Expression of the marker gene in response to induction or selection usually indicates expression of the tandem gene as well.

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein or the cell expressing the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Alternatively, the signal sequence may localize the protein to the nucleus, where for example a REST-VP16 will activate neuronal differentiation genes. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor Xa or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath et al. (1992) while the enterokinase cleavage site provides a means for purifying the desired polypeptide from the fusion protein. A discussion of vectors which contain fusion proteins is provided in Kroll et al. (1993).

1. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). The term “vector” is used to describe these nucleic acid delivery vehicles. Vector components of the present invention may be a viral vector that encode one or more candidate substance. Non-limiting examples of virus vectors that may be used to deliver a nucleic acid of the present invention are described below.

a. Adenoviral Vectors

A particular method for delivery of the nucleic acid involves the use of an adenovirus vector. Although adenovirus expression vectors are known to have a low capacity for integration into genomic DNA, this feature is counterbalanced by the high efficiency of gene transfer afforded by these vectors. “Adenovirus expression vector” is meant to include those constructs containing adenovirus nucleic acid sequences sufficient to (a) support packaging of the construct and (b) to ultimately express a tissue or cell-specific construct that has been cloned therein. Knowledge of the genetic organization of adenovirus, a 36 kb, linear, double-stranded DNA virus, allows substitution of large pieces of adenoviral DNA with foreign sequences (e.g., Grunhaus and Horwitz, 1992).

b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) vectors are an attractive vector system for use in the present invention as it has a high frequency of integration of the AAV expression vector and the vector can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

c. Retroviral Vectors

Retroviruses have promise as vectors due to their ability to integrate their genes into the host genome, transferring a large amount of foreign genetic material, infecting a broad spectrum of species and cell types and of being packaged in special cell-lines (Miller, 1992). Examples of retroviral vectors are described in Federico, 1999, which is incorporated herein by reference.

In order to construct a retroviral vector, a nucleic acid (e.g., one encoding a REST-transactivator) is inserted into the viral genome in the place of certain viral sequences to produce a virus that is replication-defective. In order to produce vinons, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

The retroviral vectors of the present invention may be readily derived from a wide variety of retroviruses, including for example, B, C, and D type retroviruses as well as spumaviruses and lentiviruses (see RNA Tumor Viruses, 1985). An example of a retrovirus suitable for use in the compositions and methods of the present invention includes, but is not limited to, lentivirus. Other retroviruses suitable for use in the compositions and methods of the present invention include, but are not limited to, Avian Leukosis Virus, Bovine Leukemia Virus, Murine Leukemia Virus, Mink-Cell Focus-inducing Virus, Murine Sarcoma Virus, Reticuloendotheliosis virus and Rous Sarcoma Virus. Particularly preferred Murine Leukemia Viruses include 4070A and 1504A (Hartley and Rowe, 1976), Abelson (ATCC No. VR-999), Friend (ATCC No. VR-245), Graffi, Gross (ATCC No. VR-590), Kirsten, Harvey Sarcoma Virus and Rauscher (ATCC No. VR-998), and Moloney Murine Leukemia Virus (ATCC No. VR-190). Such retroviruses may be readily obtained from depositories or collections such as the American Type Culture Collection (“ATCC”; Rockville, Md.), or isolated from known sources using commonly available techniques. The various genera and strains of retroviruses suitable for use in the compositions and methods are well known in the art (see, e.g., Fields (1996), see e.g., Chapter 58, Retroviridae: The Viruses and Their Replication, Classification, including Table 1, incorporated herein by reference).

Recombinant lentiviral vectors are capable of infecting non-dividing cells and can be used for both in vivo and ex vivo gene transfer and expression of nucleic acid sequences. For example, recombinant lentivirus is capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific. Lentiviral vectors are well known in the art (see, for example, Naldini et al., 1996; Zufferey et al., 1997; Blomer et al., 1997; U.S. Pat. Nos. 6,013,516 and 5,994,136).

d. Other Viral Vectors

Other viral vectors may be employed in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

e. Delivery Using Modified Viruses

A nucleic acid to be delivered may be housed within an infective virus, i.e. a vector, that has been engineered to express a specific binding ligand. The virus particle will thus bind specifically or preferentially to the cognate receptors of the target cell and deliver the contents to the cell.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Vector Delivery and Cell Transformation

Suitable methods for nucleic acid delivery for transformation of an organelle, a cell, a tissue or an organism for use with the current invention include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); receptor-mediated transfection (Wu and Wu, 1987; Wu and Wu, 1988); by microprojectile bombardment (WO 94/09699 and WO 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); and any combination of such methods. Through the application of techniques such as these, organelle(s), cell(s), tissue(s) or organism(s) may be stably or transiently transformed.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, an expression vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in at least one organism, such as a human. In certain embodiments, the organism may be, but is not limited to, a prokaryote (e.g., a eubacteria, an archaea) or an eukaryote, as would be understood by one of ordinary skill in the art (see, for example, webpage http://phylogeny.arizona.-edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli×1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas species, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, primary cells, cell lines, stem cells, progenitor cells, neural stem cells, neural progenitor cells, myoblasts, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using all or part of SEQ ID NO: 1 or SEQ ID NO:3. In still further embodiments, an expression vector may comprise an internal ribosome entry site whereas multiple transcripts or protein encoding nucleic acids may be delivered in one expression vector. The nucleic acid may encode a second therapeutic gene, marker, reproter, or a selectable marker. Altenatively, multiple expression vectors may introduced into a cell sequentially or concomitantly to provide additional beneficial characteristics to a cell. For example, a nucleic acid encoding the Nurrl protein may be used to produce a dopaminergic cell (Kim et al., 2003).

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell, e.g., visible on a gel.

In some embodiments, the expressed proteinaceous sequence forms an inclusion body which may be isolated as described above.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or by any technique that would be know to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

IV. Therapeutic Methods

In various embodiments compositions and methods as described herein are used for therapeutic purposes. In certain embodiments expression vectors or cells comprising expression vectors for a REST-transactivator can be used to supply neuronal cells to an organ in which neurons have been damaged or have been reduced in number. In certain aspects of the invention compositions of the invention may be administered as cellular therapies where an engineered cell is administered to a patient or as gene therapies where an expression cassette, when administered to a patient, transfects a cell(s) in a patients body. In other embodiments a therapeutic target, e.g., a cell, tissue or organ, may be contacted with a REST-transactivator protein.

A. Cellular Therapy

Cells expressing a REST-transactivator of the invention can also be used for tissue reconstitution or regeneration in a human patient in need thereof. The cells are administered in a manner that permits them to graft to the intended tissue site and reconstitute or regenerate the functionally deficient area.

In one example, engineered neural stem cells are transplanted directly into parenchymal or intrathecal sites of the central nervous system, according to the disease being treated. Grafts may be done using single cell suspension or small aggregates at a density of 25,000-500,000 cells per μL (U.S. Pat. No. 5,968,829). The number of cells administered may include about 1, about 10, about 100, about 1,000, about 5,000, about 10,000, about 15,000, about 20,000, about 25,000, about 50,000, about 100,000, about 200,000, about 300,000, about 400,000, about 500,000 cells/patient or more and includes any range therebetween.

Certain neural progenitor cells embodied in this invention are designed for treatment of acute or chronic damage to the nervous system. For example, excitotoxicity has been implicated in a variety of conditions including epilepsy, stroke, ischemia, Huntington's disease, Parkinson's disease and Alzheimer's disease. Certain REST-transactivator expressing cells of this invention may also be appropriate for treating dysmyelinating disorders, such as Pelizaeus-Merzbacher disease, multiple sclerosis, leukodystrophies, neuritis and neuropathies.

In certain embodiments, additional drugs such as hormones, neorotransmitters, other cell types, or small molecules that enahance or modulate the differentiation process may be used. For example, dopamine, dopamine producing cells, or dopamine secretagogues may be used in conjunction with cell therapy to modulate the phenotype of the resulting neuron.

B. Gene Therapy

The polypeptides and/or the polynucleotides that encode them, may also be employed in accordance with the present invention by expression of such polypeptides in vivo by means of gene therapy.

Thus, for example, cells from a patient may be engineered with a polynucleotide (DNA or RNA) encoding a polypeptide ex vivo or in vivo. Such methods are well-known in the art. For example, cells may be engineered by procedures known in the art by use of a viral, adenoviral, or retroviral particle containing nucleic acid(s) encoding a REST-transactivator polypeptide.

Gene therapy vectors may be administered at a dose of about 10², about 10³, about 10⁴, about 10⁵, about 10⁶, about 10⁷, about 10⁸, about 10⁹, about 10¹⁰, about 10¹¹ pfu or more including ranges therebetween.

In certain aspects, tumor cells are targets for therapeutic administration a REST-transactivator. A tumor may be derived from, but not limited to a neuron or a neuronal progenitor cell or tissue. For example, a non-limiting example is a medulloblastoma, which is believed to originate mostly from the undifferentiated neuroectodermal stem cells in the cerebellum (Eberhart and Burger, 2003; Pomeroy and Sturla, 2003; Marino et al., 2000; MacDonald et al., 2001; Pomeroy et al., 2002). Although most medulloblastomas represent undifferentiated phenotypes, others express markers representing various levels of differentiation in various cell lineages, such as neuronal and glial cells, and undifferentiated medulloblastoma cells can be further differentiated when exposed to various reagents (Eberhart and Burger, 2003; Pomeroy and Sturla, 2003; Marino et al., 2000; Li et al., 2004; Wang et al., 2003). A fraction (˜15%) of patients with sporadic medulloblastoma harbor genetic defects in the patched (PTCH) gene (Xie et al., 1997). In addition, some mutant ptc+/− mice produce medulloblastomas (Goodrich et al., 1997). The PTCH gene is an integral part of the hedgehog-hip-patched-smoothened pathway (McMahon et al., 2000), and a mutation in any of those genes may give rise to medulloblastoma (Wechsler-Reya and Scott, 2001). Mutations in the p53 and Rb pathways may also be involved in this disease (Marino et al., 2000; Wetmore et al., 2001; Krynska et al., 2000; Lee et al., 2003). Other mechanisms involving JC-virus and various other pathways regulated by Wnt, platelet derived growth factor-receptor-A, insulinlike growth factor I receptor, β-catenin, or Trk receptor tyrosine kinase, etc., also have been correlated with medulloblastoma (MacDonald et al., 2001; Lee et al, 2003; Wang et al., 2001; Nakagawara, 2001). Perhaps these observations reflect the fact that different mechanisms under different genetic backgrounds trigger medulloblastoma tumorigenesis. In certain aspects gene therapy with a REST-transactivator will abrogate tumor growth and/or induce apoptosis in tumors of the nervous system.

One challenge facing medulloblastoma research is understanding the mechanism or mechanisms that regulate most of these tumors. The inventors previous work indicated that several medulloblastoma cell lines, compared with either neuronal progenitor cells or fully differentiated neurons (Lawinger et al., 2000; Immaneni et al., 2000) overexpress REST/NRSF. REST/NRSF is a global transcriptional repressor (Shimojo and Hersh, 2004; Lunyak et al., 2004) responsible for silencing the transcription of most neuronal differentiation genes by binding to a 23-bp consensus DNA sequence, the RE1 binding site/neuron restrictive silencer (RE1/NRSE), which is present in these genes' regulatory regions. REST/NRSF is expressed in most if not all normeuronal cells in vivo (Shimojo and Hersh, 2004; Lunyak et al., 2004), but it is not expressed at high levels in differentiated neurons during embryogenesis. However, studies showed that it is expressed in certain mature neurons in adults (Shimojo and Hersh, 2004; Lunyak et al., 2004), suggesting that REST/NRSF may play a complex role that in turn may depend on its cellular and physiological environment. REST/NRSFdependent promoter repression requires interaction with several cellular cofactors, including Co-REST, mSin3A, and histone deacetylase complex (HDAC), and it requires histone deacetylase activity (Shimojo and Hersh, 2004; Lunyak et al., 2004; Ballas et al., 2001).

The inventors have shown that in transient transfection experiments, REST-VP16 operates through RE1/NRSE, competes with endogenous REST/NRSF for DNA binding, activates plasmid-encoded neuronal promoters in medulloblastoma cells and other mammalian cell types, and activates cellular REST/NRSF target genes, even in the absence of factors otherwise required to activate such genes. Furthermore, high efficiency expression of REST-VP16 mediated by Ad.REST-VP16 in medulloblastoma cells countered endogenous REST/NRSF-mediated repression of neuronal promoters, stimulated the expression of endogenous neuronal genes, and triggered apoptosis through the activation of caspase cascades. In addition, intratumoral injection of Ad.REST-VP16 in established subcutaneous medulloblastoma tumors in nude mice inhibited tumor growth. Using immunohistochemical analysis, here the inventors show that a majority of the 21 human medulloblastoma tumor specimens examined overexpressed REST/NRSF (6 strongly and 11 weakly). In contrast, adjacent normal cerebellum tissue sections were negative for REST/NRSF expression, indicating that overexpression of REST/NRSF is a new and major marker for human medulloblastoma tumors. Furthermore, Ad.REST-VP16 was found to block the intracranial tumorigenic potential of medulloblastoma cell lines and inhibited growth of established tumors in nude mice, suggesting that expression of neuronal differentiation genes in medulloblastoma cells can interfere with the cells' tumorigenic potential. Based on these studies, overexpression of REST/NRSF in neuronal cells inhibits the transcription of multiple neuronal differentiation genes, blocks these cells at a predifferentiation stage and contributes to medulloblastoma tumorigenesis.

V. Pharmaceutical Formulation

Aqueous compositions of the present invention will have an effective amount of a REST-transactivator, a nucleic acid encoding a REST-transactivator and/or a cell expressing a REST-transactivator. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for polypeptide, nucleic acid or cellular therapeutics are well known in the art. Except insofar as any conventional media or agent is incompatible with the active ingredients, its use in the therapeutic compositions is contemplated. Supplementary active ingredients, such as growtn promoters or anti-cancer agents, can also be incorporated into the compositions.

A. Parenteral Administration

The active compositions will often be formulated for parenteral administration, e.g., formulated for injection via the intravenous, intramuscular, intracranial, or even intraperitoneal routes. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

VI. Small Molecule Modulators of REST/NRSF

In other aspects, the invention includes compositions and methods for identifying REST/NRSF modulating compounds, such as, but not limited to small molecule modulators of REST/NRSF. Assays are described that can be used to identify compounds that modulate REST/NRSF. Molecules identified by the methods may be used to modulate REST/NRSF repression of neuronal genes. The modulation of REST/NRSF may be used, much like the introduction of a REST-transactivator, to regulate the differentiation of various cells into a neuron or a cell with a neuronal phenotype.

REST/NRSF modulating compounds include compounds which interact with REST/NRSF such that the activity of the REST/NRSF is modulated, e.g., enhanced or inhibited. In one embodiment, the REST/NRSF modulating compounds modulate the activity of a REST/NRSF as measured by assays known in the art or LANCE assays (e.g., see U.S. Patent Application 20030229065, which is incorporated herein by reference), such as those described herein.

The term “activity of a REST/NRSF” includes the ability of a REST/NRSF to interact with DNA, e.g., to bind to a REST/NRSF element, or to repress transcription from such a element. In one embodiment, the REST/NRSF modulating compound inhibits a particular REST/NRSF by about 10% or greater, about 40% or greater, about 50% or greater, about 60% or greater, about 70% or greater, about 80% or greater, about 90% or greater, about 95% or greater, or about 100% as compared to the activity of the REST/NRSF without the REST/NRSF modulating compound. Since REST/NRSF is a transcriptional repressor the inhibition of its repressor activity results in the de-repression or activation of a REST/NRSF regulated gene. RE1 elements may be engineered into a variety of expression constructs and used to regulate various marker genes. In one particular embodiment, the REST/NRSF containing expression cassette may be used in a bacterial or eukaryotic expression system to express a REST/NRSF protein. Screening for modulators of REST/NRSF activity may be done using REST/NRSF expressing cells or bacteria containing a REST/NRSF reporter cassette.

As used herein, the term “REST/NRSF responsive element” includes a nucleic acid sequence which can interact with promoters or enhancers and is involved in repressing transcription of a REST/NRSF regulated gene or an operon in a microbe.

A used herein, the term “microbe” includes microorganisms that may express or may be made to express a REST/NRSF protein. Preferably microbes are unicellular and include bacteria, fungi, or protozoa.

Test compounds that can be tested in the subject assays include various small molecules or small molecule libraries. The term “test compound” includes any reagent or test agent which is employed in the assays of the invention and assayed for its ability to influence the activity of a REST/NRSF protein, by for example, binding to the REST/NRSF polypeptide and/or to a molecule with which it interacts. More than one compound, e.g., a plurality of compounds, can be tested at the same time for their ability to modulate the activity of REST/NRSF. Exemplary test compounds which can be screened for activity include, but are not limited to, peptides, non-peptidic compounds, nucleic acids, carbohydrates, small organic molecules (e.g., polyketides), and natural product extract libraries. The term “non-peptidic test compound” includes compounds that are comprised, at least in part, of molecular structures different from naturally-occurring L-amino acid residues linked by natural peptide bonds. However, “non-peptidic test compounds” also include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds. “Non-peptidic test compounds” also are intended to include natural products. The term “antagonist” includes REST/NRSF modulating compounds, which inhibit the activity of REST/NRSF by binding to and inactivating REST/NRSF, by binding to a nucleic acid target with which the REST/NRSF interacts (e.g., RE1/NRSE element), and/or by disrupting a critical protein-protein interaction. REST/NRSF modulators may include, for example, naturally or chemically synthesized compounds such as small cell permeable organic molecules, nucleic acid interchelators, peptides, and the like.

Nucleic acids encoding REST/NRSF can be expressed in cells using vectors. Almost any conventional delivery vector can be used. Such vectors are widely available commercially and it is within the knowledge and discretion of one of ordinary skill in the art to choose a vector which is appropriate for use with a given expression system. The sequences encoding these domains can be introduced into a cell on a self-replicating vector or may be introduced into the chromosome of a cell by recombination or by an insertion element such as a transposon. These nucleic acids can be introduced into cells using standard techniques, for example, by transformation using calcium chloride or electroporation. Such techniques for the introduction of DNA into cells are well known in the art.

In one embodiment, the invention provides for methods of identifying a test compound which modulates the activity of a REST/NRSF protein by contacting a cell expressing a REST/NRSF protein (or portion thereof) with a test compound under conditions which allow interaction of the test compound with the cell. The ability of the test compound to modulate an activity of REST/NRSF can be determined in a variety of ways.

Various assays may be used to assess or determine the modulatory characteristics of test compounds. In one embodiment, the expression of a selectable marker that confers a selective growth advantage or disadvantage, or lethality is placed under the control of a RE1 element in a cell expressing REST/NRSF. A REST/NRSF plasmid may be encoded in a cell's genome or be episomal. In one embodiment, the genetic background of the host is manipulated. REST/NRSF may be expressed endogenously, exogenously and/or constitutively in a cell or system used for screening.

In one embodiment, a selective marker is a cytotoxic gene product (e.g., ccdb). In another embodiment, a selective marker is a gene that confers antibiotic resistance (e.g., kan, cat, or bla). In still another embodiment, a selective marker is a gene that confers a selective growth disadvantage in the presence of a particular metabolic substrate (e.g., the expression of URA3 in the presence of 5-fluoroorotic acid (5-FOA) in yeast).

Compounds that modulate REST/NRSF may be identified using a one-hybrid screening assay. As used herein, the term “one-hybrid screen” includes assays that detect the disruption of protein-nucleic acid interactions. These assays will identify agents that interfere with the binding of a REST/NRSF to a particular target by binding to the target and preventing the REST/NRSF from interacting with or binding to this site.

In another embodiment, compounds of the invention may be identified using a two-hybrid screening assay. As used herein the term “two-hybrid screen” as used herein includes assays that detect the disruption of protein-protein interactions. Such two hybrid assays can be used to interfere with crucial protein-REST/NRSF interactions.

In one embodiment of the assay, the expression of a selective marker (e.g., ccdb, cat, bla, kan, guaB, URA3) is put under the direct control of a REST/NRSF repressed promoter. In the absence of REST/NRSF or the presence of an inhibitor of REST/NRSF activity, the selective marker will be expressed. For example, in the case of regulation of the cytotoxic gene ccdb, the gene would be expressed and the cells would not survive. Addition of REST/NRSF would result in the repression of the promoter and the reduced expression of the selective marker. In the case of ccdb, the gene would not be expressed and result in cell survival. Compounds that inhibit REST/NRSF would be identified as those that induce cell death in the presence of REST/NRSF.

In another embodiment, for example, where the promoter regulates a gene such as URA3, a different result could be obtained. In this case, in the absence of REST/NRSF activity and thus, in the presence of URA3 expression, cells would be growth inhibited following the conversion of 5-FOA to a toxic metabolite by URA3. Upon repression of expression and thus reduction of URA3 synthesis, cells would grow. Compounds that inhibit REST/NRSF would be identified as those that induce cell growth inhibition in the presence of REST/NRSF.

Candidate compounds for testing in the instant methods can be derived from a variety of different sources and can be known or can be novel. In one embodiment, libraries of compounds are tested in the instant methods to identify REST/NRSF modulating compounds.

A recent trend in medicinal chemistry includes the production of mixtures of compounds, referred to as libraries. While the use of libraries of peptides is well established in the art, new techniques have been developed which have allowed the production of mixtures of other compounds, such as benzodiazepines (Bunin et al., 1992; DeWitt et al., 1993), peptoids (Zuckennann, 1994), oligocarbamates (Cho et al., 1993), and hydantoins (DeWitt et al., 1993). The synthesis of molecular libraries of small organic molecules with a diversity of 10⁴-10⁵ have been described (Carell et al. 1994).

The compounds of the present invention may be obtained using any of the numerous approaches in combinatorial library methods known in the art, including: biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, 1997).

Exemplary compounds which can be screened for activity include, but are not limited to, peptides, nucleic acids, carbohydrates, small organic molecules, and natural product extract libraries. In one embodiment, the test compound is a peptide or peptidomimetic. In another, preferred embodiment, the compounds are small, organic non-peptidic compounds. Other exemplary methods for the synthesis of molecular libraries can be found in the art, for example in: Erb et al. (1994); Horwell et al. (1996); and in Gallop et al. (1994).

Libraries of compounds may be presented in solution (e.g., Houghten, 1992), or on beads (Lam, 1991), chips (Fodor, 1993), bacteria (Ladner, U.S. Pat. No. 5,223,409), spores (Ladner U.S. patent '409), plasmids (Cull et al., 1992), or on phage (Scott and Smith, 1990); (Devlin, 1990); (Cwirla et al., 1990); (Felici, (1991). Other types of peptide libraries may also be expressed, see, for example, U.S. Pat. Nos. 5,270,181 and 5,292,646, which are incorporated herein by reference. In still another embodiment, combinatorial polypeptides can be produced from a cDNA library.

In yet another embodiment, computer programs can be used to identify individual compounds or classes of compounds with an increased likelihood of modulating a REST/NRSF activity. Such programs can screen for compounds with the proper molecular and chemical complementarities with a chosen REST/NRSF. In this manner, the efficiency of screening for REST/NRSF modulating compounds in the assays described above can be enhanced.

Computer modeling techniques may be utilized for identifying REST/NRSF modulating compounds. Molecular design techniques may be used to design REST/NRSF modulating compounds, which are capable of binding or interacting with one or more REST/NRSF polypeptides.

In an embodiment, the invention pertains to a method of identifying REST/NRSF modulating compounds. The method includes obtaining or modeling the structure of REST/NRSF or a variant thereof, and using GLIDE to identify a scaffold which has an interaction energy score of −20 or less (e.g., −40 or less, e.g., −60 or less) with a portion of REST/NRSF.

The invention pertains, at least in part, to a computational screening of small molecule databases for chemical entities or compounds that can bind in whole, or in part, to REST/NRSF. In this screening, the quality of fit of such entities or compounds to the binding site may be judged either by shape complementarity or by estimated interaction energy (Meng et al., 1992). Such a procedure allows for the screening of a very large library of potential REST/NRSF modulating compounds for the proper molecular and chemical complementarities with a selected protein or class or proteins. REST/NRSF modulating compounds identified through computational screening can later be passed through in vitro and in vivo assays as further screens.

A variety of crystal structures are available in the Protein Data Bank (www.rcsb.org/pdb/). These structures may be used as model structures to identify sites on the proteins that could be targeted by small molecule chemical inhibiting compounds.

The GLIDE docking method may then be used to fit combinatorial chemistry scaffolds into these sites and an interaction energy was calculated for each. These scaffolds may then be used to search, for example, the CambridgeSoft ACX-SC database of over 600,000 non-proprietary chemical structures and the number of chemicals similar to the scaffolds. The term “scaffold” includes the compounds identified by the computer modeling program. These compounds may or may not be themselves REST/NRSF modulating compounds. An ordinarily skilled artisan will be able to analyze a scaffold obtained from the computer modeling program and modify the scaffold such that the resulting compounds have enhanced chemical properties over the initial scaffold compound, e.g., are more stable for administration, less toxic, have enhanced affinity for a particular REST/NRSF. The invention pertains not only to the scaffolds identified, but also the REST/NRSF modulating compounds which are developed using the scaffolds.

A REST/NRSF modulating compound or other binding compound may be computationally evaluated and designed by screening and selecting chemical entities or fragments for their ability to associate with the individual small molecule binding sites or other areas of REST/NRSF or variant thereof.

Specialized computer programs may also assist in the process of selecting molecules that bind to REST/NRSF. The programs include, but are not limited to GRID (Goodford, 1985); AUTODOCK (Goodsell and Olsen, 1990); MCSS (Miranker and Karplus, 1991); MACCS-3D (Martin, 1992); DOCK (Kuntz et al., 1982); and/or MCDLNG (Monte Carlo De Novo Ligand Generator) (Gehlhaar et al., 1995).

Once suitable chemical fragments have been selected, they can be assembled into a single compound or inhibiting compound. Assembly may be proceed by visual inspection of the relationship of the fragments to each other on a three-dimensional image display on a computer screen in relation to the structure coordinates of a particular REST/NRSF or REST/NRSF model. This may be followed by manual model building using software such as Quanta or Sybyl.

Useful programs to aid one of skill in the art in connecting the individual chemical fragments include: 3D Database systems such as MACCS-3D (MDL Information Systems, San Leandro, Calif.; reviewed in Martin (1992); CAVEAT (Bartlett et al., 1989); and/or HOOK (Molecular Simulations, Burlington, Mass.).

In another embodiment, REST/NRSF modulating compounds may be designed as a whole or “de novo” using either an empty active site or optionally including some portion(s) of a known inhibiting compound(s). These methods include: LUDI (Bohm, 1992); Biosym Technologies, San Diego, Calif.; LEGEND (Nishibata and Itai, 1991); Molecular Simulations, Burlington, Mass.; CoMFA (Conformational Molecular Field Analysis) (Kaminski. 1994); LeapFrog (Tripos Associates, St. Louis, Mo.); and/or FlexX (D 1993-2002 GMD German National Research Center for Information Technology; Rarey et al., 1999). Other molecular modeling techniques may also be employed in accordance with this invention. See, e.g., Cohen et al. (1990) and Navia and Murcko (1992).

Once a compound has been designed and selected by the above methods, the efficiency with which that compound may bind to a particular REST/NRSF may be tested and optimized by computational evaluation.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa. © 1992); AMBER, version 4.0 (P. A. Kollman, University of California at San Francisco, ©1994); QUANTA/CHARMM (Molecular Simulations, Inc., Burlington, Mass. ©1994); and Insight II/Discover (Biosysm Technologies Inc., San Diego, Calif. ©1994). These programs may be implemented, for instance, using a Silicon Graphics workstation, IRIS 4D/35 or IBM RISC/6000 workstation model 550. Other hardware systems and software packages will be known to those skilled in the art.

Once a REST/NRSF modulating compound has been optimally selected or designed, as described above, substitutions may then be made in some of its atoms or side groups in order to improve or modify its binding properties. Initial substitutions are preferable conservative, i.e., the replacement group will have approximately the same size, shape, hydrophobicity and charge as the original group. Such substituted chemical compounds may then be analyzed for efficiency of fit to REST/NRSF by the same computer methods described above. VII. KITS

All the essential materials and reagents required for administering a protein therapy, a gene therapy or a cellular therapy by a particular method discussed above may be assembled together in a kit. When the components of the kit are provided in one or more liquid solutions, the liquid solution preferably is an aqueous solution, with a sterile aqueous solution being particularly preferred.

Various components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet outlining suggested polypeptide, nucleic acid or cell dosages when a particular disease state is identified in a patient.

The kits of the present invention also will typically include a means for containing the vials in close confinement for commercial sale such as, e.g., injection or blow-molded plastic containers into which the desired vials are retained.

### EXAMPLES

Demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Conversion of Myoblasts to Physiolocally Active Neuronal Phenotype

A. Materials and Methods

Plasmids. The NheI/Xho I fragment of pcDNA3.1-REST-VP16 (Immaneni et al., 2000) was subcloned into the NheI/Xho I digested plasmid pBig2r (Starthdee et al., 1999). The clone obtained was confirmed by sequencing the junction region. Construction of pNaCh, pNaChARE1, pREST/NRSF, pGal4-VP16, pREST-VP16pTLuc and pRE.TLuc has been described elsewhere (Immaneni et al., 2000; Lawniger et al., 1999; Watanabe et al., 2004). The pCMV-pGAL was purchased from Stratagene Co. (La Jolla, Calif.)

Cell culture. The mouse myoblast C2C12 cells were purchased from American Type Culture Collection (Manalssas, Va.). C2C12 cells were maintained in Minimal Essential Medium (Invitrogen Co., Carlsbad, Calif.) containing 10% fetal calf serum (FCS) at 37° C. in a humidified atmosphere of 5% CO₂ in air. The cells were not grown to confluence. To induce muscle differentiation, the cells were grown to subconfluency in 10% fetal calf serum-Dulbeccos Modified Eagle's Medium (DMEM), and then the medium was changed to DMBM containing 2% horse serum (Invitrogen). The medium was changed at least every 2 days. Culture vessels coated with poly-1-lysine (final concentration of 20 pg/ml in distilled water) (Sigma, St. Louis, Mo.) were used in the assays. For both calcium imaging and FM1-43 staining cells were differentiated for 10 days on glass cover slip coated with 0.005% poly-L-lysine and 20 μg/ml laminin.

Stable transfection. C2C12 cells were plated into 60-mm plates at a density of 5×10 per dish the day before the transfection. Two milligrams of pBig2r-REST-VP16 was transfected with 6 μl of Fugene 6 (Roche Diagnostics Co., Indianapolis, Ind.) according to the manufacturer's instructions. Doxycycline (2 μg/ml) (Sigma) was added to the culture and refreshed every 2 days. After 10 days of hygromycin B (750 μg/ml) (Roche) selection, colonies were picked and expanded. Doxycycline-inducible clones were selected by induction of REST-VP16 two days after the removal of doxycycline and identified by Western blot analysis. Two clones (clones 2 and 7) expressed high levels of REST-VP16. After selection, clones were maintained in medium containing 500 μg/ml hygromycin B. During the assays, the concentration of hygromycin B was reduced to 250 μg/ml to avoid the removal of the transgene.

SDS-polyacrylamide gel electrophoresis (PAGE) and Western blotting. Cells were treated, rinsed, and lysed directly in Laemmli sample buffer (Bio-Rad, Hercules, Calif.). The lysates were mixed vigorously in a vortex mixer, boiled for 5 minutes and subjected to SDS-PAGE. Western blotting was performed using Hybond-P PVDF membranes (Amersham Biosciences, Buckinghamshire, UK) and ECL-plus reagent (Amersham).

Antibodies. The antibodies used were anti-VP16 (1:1000) (Clontech, Palo Alto, Calif.); anti-synapsin I (1:1000) (MAB355; Chemicon, Temecula, Calif.); anti-actin (1:2000) (1-19; Santa Cruz Biotech., Inc., Santa Cruz, Calif.); anti-MAP2 (1:10,000 for IF, 1:1000 for Western) (HM-2; Sigma); anti-unique β-tubulin (βIII) (1:1000) (Tujl; Covance Research Products, Inc.); anti-α-tubulin (1:10,000) (B-5-1-2; Sigma); anti-myosin heavy chain (1:10) (MF20; Developmental Studies Hybridoma Bank, Iowa City, Iowa); antisecretogranin II antibody (1:100) (QED Bioscience); anti-synaptophysin I (1:200) (Chemicon, Cat #mab368); anti-synaptotagmin (1:200) (Chemicon, Cat # AB5600); anti-bromodeoxyuridine (BrdU) (6 μg/ml) (BMC9318; Roche); horseradish peroxidase-conjugated anti-mouse, anti-rabbit IgG (H+L) (1:20,000) (Amersham); HRP-conjugated anti-goat IgG (1:20,000) (Santa Cruz); fluorescein anti-rabbit IgG (1:250) (Vector Lab., Inc., Burlingame, Calif.); and Cy2-labelled anti-mouse IgG (H+L) (1:1000) (Amersharn). For Western blotting with neurofilament, anti-neurofilament-I 601200 kD (1:50 dilution, RMdO-20, Zymed); Nti-GFAP. (DAKO, 1:500); anti-myogenin (Santa Cruz, 1:200) antibodies were used.

Reporter gene assays. C2C12 cells and C2C12-RV clone cells were plated into poly-1-lysine-coated 24-well plates at a density of 8×10⁴ cells per well and 1×10⁵ cells per well, respectively, the day before transfection. A combination of 100 ng of pTLuc, pRE.Tluc, or pcDNA3.1 and 10 ng of pCMV-PGAL was transfected with Lipofectamine 2000 reagent (Invitrogen) according to the manufacturer's instructions. During the transfection, doxycycline was removed from the culture. Six hours after the transfection, the cells were trypsinized, divided into 2 wells of a 6-well plate with or without doxycycline, and cultured for 42 hours. The harvested cells were assayed for luciferase and β-galactosidase as described previously (Immaneni et al., 2000; Lawinger et al., 2000).

Immunofluorescence analysis. Cells were fixed for 20 to 30 min at room temperature with either 4% paraformaldehyde in phosphate-buffered saline (PBS) or 10% buffered formalin, rinsed in PBS, permeabilized, and blocked in blocking buffer (5% nonfat dry milk (Nestle), 0.3% Triton X-100 (Sigma), and PBS) for 30 minutes at room temperature. Primary antibodies were diluted in the blocking buffer and incubated overnight at 4° C. in humidified conditions. Secondary antibodies were diluted in PBS and incubated for 1 hour at RT. For nuclear staining, Hoechst 33328 (Molecular Probes) was used at 0.1 μg/ml in H₂O. Photographs were taken with a Hamamatsu 5880 color CCD camera with a Leica DMR microscope. Double-label images were assembled in Adobe Photoshop. Alternatively, stained cells were covered with a coverslip. Staining of cells was observed using a Leica Epifluorescence microscope.

Mitotic inhibition studies. Cells were cultured for 6 days in 2% horse serum-DMEM and then for 14 days in inhibitor medium (2% horse serum-DMEM and mitotic inhibitors). The final concentrations of inhibitors were 10 μM 5-fluoro-2′-deoxyuridine, 10 μM uridine, and 1 μM cytosine β-d-arabinofuranoside (Sigma).

Gene profiling assay. Total RNA was extracted from C2C12 myoblast cells, C2C12(D) muscle cells and C2C12-RV(D) neuronal cells using a Qiagen RNA extraction kit (Valencia, Calif.) and was subjected to Affymetrix U74Av2 mouse chips. The data was quantified using Affymetrix Microarray Analysis Suite (MAS) 5.0. Estimates of signal intensity and detection p-values were loaded into S-Plus for further analysis. A detection filter was applied to determine which genes should be retained for further analysis. Genes were retained that had a detection p-value less than 0.05 on at least one array. Of the 12,488 probe sets on the array, 7,496 passed this filter. The remaining 4,992 probe sets were dropped. The data was log transformed and a hierarchical cluster analysis was performed to determine which pair of samples was most similar. Average linkage and a similarity measure based on the Pearson correlation coefficient was used. By relying on the values from individual probe pairs, MAS 5.0 to was used to perform a Wilcoxon signed rank test to compare genes from two chips. Pair-wise comparisons were carried out in this manner for each of the three desired comparisons. A summary of the to upregulated genes in C2C12-RV(D) cells compared to C2C12(D) cells and the rop upregulated genes in C2C12(D) cells compared to C2C12-RV(D) cell are shown in Table 2 and Table 3, respectively.

TABLE 2
List of top upregulated genes in C2C12-RV(D) compared to
C2C12(D)

| | Fold |
|---|---|
| | difference |
| Name of Gene | (x2) |
| chromogranin C (Secretogranin II) | 5.75 |
| chromogranin A | 3.09 |
| chromogranin B | 2.88 |
| synaptosomal-associated protein, 25 kDa | 2.83 |
| neuronal tyrosine/threonine phosphatase 1 | 2.60 |
| leucine rich repeat protein 1, neuronal | 2.14 |
| potassium voltage gated channel, Shaw-related subfamily, | 1.80 |
| member 1 | |
| microtubule-associated protein tau | 1.74 |
| nerve growth factor, beta | 1.63 |
| piccolo (presynaptic cytomatrix protein) | 1.60 |
| POU domain, class 2, transcription factor 1 | 1.48 |
| Neuromedin | 1.46 |
| myosin Va | 1.33 |
| tubulin, beta 3 | 1.29 |
| solute carrier family 6 (neurotransmitter transporter, taurine), | 1.28 |
| member 6 | |
| v-maf musculoaponeurotic fibrosarcoma oncogene family, | 1.23 |
| protein B (avian) | |
| Calcium channel, voltage-dependent, gamma subunit 6 | 1.14 |
| synaptotagmin 10 | 1.13 |
| tubulin, beta 5 | 1.11 |
| melatonin receptor 1A | 1.10 |
| solute carrier family 12, member 7 | 1.08 |
| inositol 1,4,5-triphosphate receptor 2 | 1.01 |
| ectodermal-neural cortex 1 | 0.91 |
| glycine receptor, alpha 1 subunit | 0.86 |
| glutamate receptor, ionotropic, NMDA2C (epsilon 3) | 0.85 |
| Chloride channel calcium activated 1 | 0.84 |
| POU domain, class 5, transcription factor 1 | 0.83 |
| synaptosomal-associated protein, 23 kD | 0.78 |
| adrenergic receptor, alpha 2c | 0.76 |
| potassium voltage-gated channel, shaker-related subfamily, | 0.73 |
| beta member 1 | |
| neuropeptide Y receptor Y2 | 0.72 |
| sodium channel, voltage-gated, type VI, alpha polypeptide | 0.70 |
| homer, neuronal immediate early gene, 1 | 0.63 |
| potassium voltage-gated channel, shaker-related subfamily, | 0.56 |
| beta member 2 | |
| 5-hydroxytryptamine (serotonin) receptor 3A | 0.54 |
| cholinergic receptor, nicotinic, alpha polypeptide 1 (muscle) | 0.52 |
| retinoblastoma 1 | 0.50 |
| cholinergic receptor, muscarinic 1, CNS | 0.46 |
| glutamate receptor, ionotropic, kainate 1 | 0.41 |
| calbindin 2 | 0.40 |
| synaptobrevin like 1 | 0.39 |
| potassium voltage-gated channel, shaker-related subfamily, | 0.34 |
| member 2 | |
| solute carrier family 12, member 4 | 0.32 |
| sonic hedgehog homolog, (Drosophila) | 0.31 |
| myosin VI | 0.27 |
| dopamine receptor 4 | 0.21 |

TABLE 3
List of top upregulated genes in C2C12(D) compared to C2C12-
RV(D)

| | Fold |
|---|---|
| | difference |
| Name of Gene | (x2) |
| procollagen, type I, alpha 1 | 6.58 |
| myosin, heavy polypeptide 1, skeletal muscle, adult | 4.00 |
| myosin heavy chain 11, smooth muscle | 2.95 |
| calsequestrin 1 | 2.79 |
| procollagen, type I, alpha 1 | 2.72 |
| Inhibitor of DNA binding 2 | 2.68 |
| Troponin C, fast skeletal | 2.32 |
| myosin, heavy polypeptide 8, skeletal muscle, perinatal | 2.25 |
| myosin light chain, alkali, cardiac ventricles | 2.08 |
| Inhibitor of DNA binding 1 | 1.96 |
| procollagen, type VI, alpha 1 | 1.90 |
| procollagen, type VI, alpha 2 | 1.85 |
| cofilin 2, muscle | 1.82 |
| myosin heavy chain, cardiac muscle, fetal | 1.72 |
| procollagen, type III, alpha 1 | 1.70 |
| Inhibitor of DNA binding 3 | 1.62 |
| selenoprotein W, muscle 1 | 1.58 |
| MyoD family inhibitor | 1.57 |
| Myoglobin | 1.43 |
| procollagen, type I, alpha 2 | 1.30 |
| ATPase, Ca++ transporting, cardiac muscle, fast twitch 1 | 1.29 |
| MyoD family inhibitor | 1.27 |
| actin, alpha, cardiac | 1.21 |
| procollagen, type VII, alpha 1 | 0.78 |
| Osteoglycin | 0.69 |
| transforming growth factor, beta induced, 68 kDa | 0.64 |
| procollagen, type XI, alpha 2 | 0.58 |
| myosin light chain, phosphorylatable, fast skeletal muscle | 0.53 |
| Troponin I, skeletal, fast 2 | 0.45 |
| Vimentin | 0.29 |
| procollagen, type XI, alpha 2 | 0.28 |
| procollagen, type VI, alpha 3 | 0.27 |
| myosin heavy chain, cardiac muscle, adult | 0.23 |
| procollagen, type I, alpha 2 | 0.14 |
| procollagen, type V, alpha 1 | 0.13 |

RT-PCR assay. C2C12 or C2C12RV cells were seded at a density of 5×10⁵ cells per 10 cm petridish coated with 0.1 mg/ml ploylysine and 0.1 μg/ml of laminin. The cells were grown in DMEM containing 2% horse serum at 37° and 5% CO₂ tension. At the end of 10 days, cells wre harvested using a cell scraper and washed with 1×PBS. Total RNA was preparted from these cells using the RNeasy Kit from Quiagen. RNAs were quantitated specrphotometically and 100 ng of total RNA per sample was used for RT-PCR. Primers used in these reactions had the folloeing sequence: Mouse LRNN1-S585-604 5′-CCGGAARRCTTTTCCAGTGA-3′ (SEQ ID NO:6); mouse LRRN1-AS815-796: 5′-AAACACAAAGCTGGGGACAC-3′ (SEQ ID NO:7); mouse SCG10-S1009-1029: 5′-TCCAACCGAAAAATGAGGTC-3′ (SEQ ID NO:8); mouse SCG10-AS1177-1158: 5′-GGCAGGAAGCAGATTACGAG-3′ (SEQ ID NO:9); mouse BDNF-S3612-3631: 5′-TTGTTTTGTGCCGTTTACCA-3′ (SEQ ID NO:10); mouse BDNF-AS3842-3722: 5′-GGTAAGAGAGCCAGCCACTG-3′ (SEQ ID NO:11); mouse SYN1-S1127-1146: 5′-TGTCGGGTAACTGGAAGACC-3′ (SEQ ID NO:12); mouse SYN1-AS1360-1341: 5′-AGTTCCACGATGAGCTGCTT-3′ (SEQ ID NO:13); mouse P-Actin AS-841: 5′-CGCTCGTTGCCAATAGTGATGACCTG-3′ (SEQ ID NO:14); and mouse β-actin S-57: 5′-GTCCACACCCGCCACCAGTT-3′ (SEQ ID NO:15). The initial reverse transcription reaction was carried out using the RT-PCR kit as per manufacturer's instructions (Qiagen). Thereafter, the cDNA was amplified for 25 cycles under the following conditions: Melting at 94° C. for 1 minute, annealing at 55° C. for 1 minute and extension at 72° C. for 1 minute. The reaction products were analyzed with 2% agarose gel electrophoresis.

Calcium Imaging. Living-cell microscopy to detect depolarization-dependent calcium influx was carried out according as described (Pincus et al., 1998; Roy et al., 2000; Bischofberger and Schild, 1995). A confocal laser system (Olympus Fluoview FV500) and the fluorescent calcium indicator Fluo-3 (Molecular Probe) were used to measure the intracellular free calcium concentration. Cell was loaded with Fluo-3 and depolarized with High KCl (100 mM) Ringer solution as previously described (Bischofberger and Schild, 1995). Fluorescence was measured every 0.25s for a total of 5s. For the glutamate experiment, 10 μM glutamate was included in Ringer solution.

FM1-43 staining. Cells were loaded with 15 μM FMI-43 (Molecular Probe, Oreg.) dissolved in High KCl (100 mM) Ringer (external [Na⁺] was reduced by an equivalent amount) for 5 min at room temperature, and washed in Ringer solution without a dye for 15 min to remove superficial FM1-43. A dye was excited at 470 nm and the emitted fluorescence was collected at 540 nm.

Cerebellar inoculation of cells into mice and assay of neuronal phenotype. These studies were performed following M.D. Anderson Institutional Animal Care and Use guidelines. C2C12 and C2C12-RV(D) (−Dox) cells were centrifuged and 2×10⁵ cells present in 5 μl cell growth medium were inoculated in a group of 6 mice for each condition using an implantable guide-screw system described elsewhere (Lal et al., 2000). This method, which uses a guide screw, a stylet, a modified Hamilton syringe with a 26-gauge needle, and an infusion pump, allows delivery of cells to a very specific location in the brain. Several days before the inoculation, the guide screws were placed in the dorsal cranium of mice. The animals were anesthetized by intraperitoneal injection of 1.2% avertin solution made in sterile PBS per gram of body weight (100% avertin solution: 10 g tribromoethylalcohol+10 ml tertiary amyl alcohol) at a dosage of 15-17 μl per gram of body weight. The guide screw entry site was marked at a point 2.5 mm lateral and 1 mm anterior to the bregma, which is located directly above the caudate nucleus. A small, hand-controlled twist drill was used to make the hole, and a specially devised screwdriver was used to thread the screw into the hole and secure it. After a recovery period of 5 or 7-10 days after the guide screws were placed, the cells were inoculated. On the day of implantation, the cells were harvested and resuspended in Ca⁺- and Mg⁺-free PBS. The cells were kept on ice until implantation. Mice with the guide screws in place were reanesthetized as described above. The tumor cell suspension was drawn into a Hamilton syringe, which was fitted with a cuff to control the depth of injection. The needle of the Hamilton syringe was slowly lowered into the center of the guide screw until the cuff rested on the screw surface. Up to 10 mice attached to 10 syringes were positioned in a row in an Harvard Apparatus (Holliston, Mass.) infusion pump that provided continuously controlled slow injection of the cells into the caudate nucleus at a rate of 1 μl/min. After the entire volume of the cell suspension was injected, the needles were manually removed, and with a fine forceps, the stylets were positioned in the screw holes to close the system and prevent the injected cells from leaking after inoculation.

Immunohistochemical double staining of mouse brain paraffin sections. The mice were sacrificed by CO₂ inhalation and further procedures were performed as previously described (Simmons et al., 2001; Lawinger et al., 2000). Briefly, mouse brains were fixed in neutral buffered formalin, dehydrated and embedded in paraffin. Five-micron sections were cut onto charged glass slides and used for light microscopic evaluation after staining by hematoxylin-eosine (H&E). For immunostaining, sections were deparaffinized and rehydrated in graded ethanols to phosphate buffered saline (PBS). Endogenous peroxidase activity was blocked using 3% hydrogen peroxide for 30 min at room temperature. Blocking buffer (10% bovine serum in PBS) was applied for 30 min at room temperature in a humidified chamber. Anti-VP-16 antibody (Clonetech) was used as a 1:50 dilution in blocking buffer overnight at 4° C. Sections were washed 3 times in PBS the following day. Conjugated anti-rabbit antibody (Envision kit, Dako) as applied for 30 min at room temperature. After washing, the staining was visualized using DAB as a chromogenic substrate (Dako) according to the manufacturer's instructions. The sections were mounted for visualization of staining. The cover slip was them removed and the sections rehydrated. Antigen retrieval was performed in 10 mM sodium citrate, pH 6.0 using a microwave-pressure cooker (Kmart) for 10 min at high power, followed by 12 min at 20% power. The slides were cooled for 30 min, washed in PBS and incubated in blocking buffer for 30 min at room temperature. An antibody to neuronal class III β-tubulin (TuJ1, Covance) was diluted 1:10 in blocking buffer and added to the slides. Following an overnight incubation at 4° C. in a humidified chamber, the slides were washed in PBS. Biotinylated anti-mouse antibody (Vector labs) was added for 30 min at room temperature. The “ABC” reagent was then added and the substrate was developed using a blue chromogen (Vector VIP kit) according to the manufacturer's instructions. Following dehydration in graded ethanols, the slides were mounted and photographed. Specimens were then examined under light microscope and photographed.

Staining with Alexa 647 conjugated bungarotoxin to label neuromuscular junctions. C2C12 myoblasts (10⁵ cells) were seeded onto 1 cm glass coverslips previously coated with 0.1 mg/ml polylysine (Sigma) and 0.1 μg/ml laminin (Invitrogen) and incubated overnight at 37° C. and 5% CO₂ in DMEM (GIBCO) supplemented with 20% fetal bovine serum, 2 mM glutamine and antibiotics. The growth medium was removed 24 hours later and replaced with DMEM containing 2% horse serum, 2 mM Gln and antibiotic to promote differentiation into muscle cells.

Upon myotube formation 96-120 hours later, 10⁵ C2C12RV cells were seeded on the layer of differentiated muscles and grown in DMEM containing 2% horse serum, 2 mM Gln and antibiotic. These conditions allow expression of the REST-VP16 transgene in C2C12RV cells and promote differentiation into neuronal cells. The cells were incubated under these conditions for 12-25 days and then stained with alexa 647-conjugated α-bungarotoxin (Molecular Probes) to visualize formation of neuromuscular junctions. Muscle cells grown in the absence of C2C12RV cells were used as controls.

Both C2C12 myotubes and C2C12 myotube-C2C12RV neuron co-cultures were stained with a solution of alexa 647-conjugated α-bungarotoxin in 1×PBS, pH 7.2, at a final concentration of 80 nm for 30 minutes in the dark. Unbound toxin was removed by 3 washes with 1×PBS, pH 7.2. The cells were fixed with 2% EM grade formaldehyde for 10 minutes, washed with PBS and covered with 50% buffered glycerol. Stained cells were observed under a Zeiss LSM 510 confocal microscope.

Transfection of REST-VP16 into Human Mesenchymal Stem Cell (hMSC). Purified HMSC cells were purchased from Cambrex and seeded at 5000 cells per chamber of CC2-coated 4-well chamber slides in MSCGM (mesenchymal stem cell growth medium, provided by Cambrex)) 24 hrs before transfection. The culture medium was changed to MSCBM (mesenchymal stem cell basal medium), which is a serum-free medium. Cells were then transfected with 1.2 μg of DNA (either control pcDNA 3.1 vector DNA or pcDNA 3.1-REST VP16 DNA. After 4 to 6 hrs, the media was changed back to MSCGM. After 24 hours, the cells were fixed with paraformaldehyde. The cells were subjected to immunocytochemistry with antibody against neuronal β-tubulin (anti-TUJ1 antibody; Convance at a dilution of 1:500 and goat anti-mouse antibody Alexa 488 (1:1000) as secondary antibody). The cell-nuclei were stained with 1 ug/ml DAPI (Molecular Probe). Cells were observed using a Leica Epifluorescence microscope.

B. Results

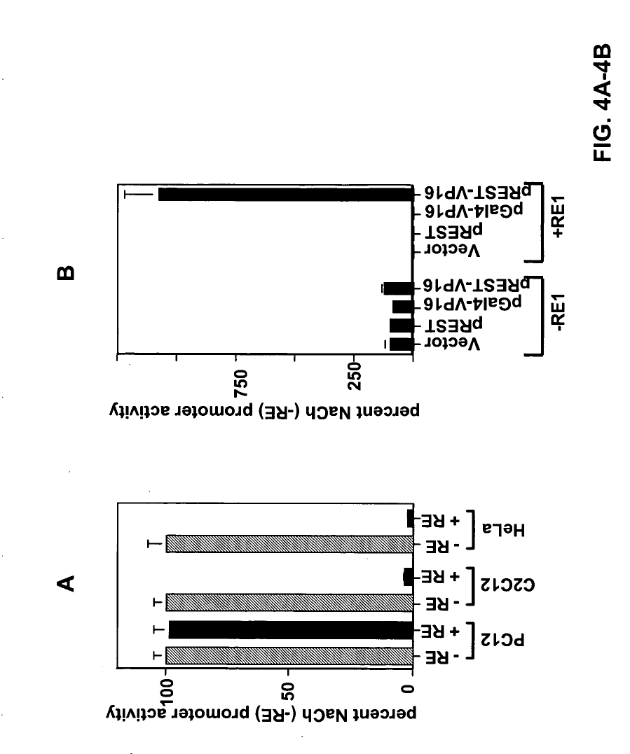

REST-VP16 counters endogenous REST/NRSF activity in myoblasts. To determine whether C2C12 cells express endogenous REST/NRSF activity and, if present, also to quantify this activity, two reporter plasmid DNAs were used (Immaneni et al., 2000; Lawinger et al., 2000). The first is pNaCh (also called pSDK7), which contains the bacterial chloramphenicol acetyl transferase (CAT) reporter gene under the control of sodium channel type II promoter/enhancer (NaCh) elements (Chong et al., 1995). The NaCh elements are mammalian native neuronal promoter/enhancer sequences that contain the REST/NRSF binding site (+RE) and are acted on by functional REST/NRSF (Chong et al., 1995). The second reporter plasmid, pNaChΔRE1 (also called pMB4), contains the CAT reporter gene under the control of the “minimal” NaCh elements without the RE1 sequence (−RE). Functional REST/NRSF does not repress the promoter activity from this plasmid (Chong et al., 1995). Thus, when functional REST/NRSF is present in the cell, the CAT activity from pNaCh (+RE) would be lower than that from pNaCh (−RE). In this experiment, PC12 cells were used, which do not express endogenous REST/NRSF, and HeLa cells, which express endogenous REST/NRSF (Chong et al., 1995; Schoenherr and Anderson, 1995; Immaneni et al., 2000; Lawinger et al., 2000). These cells were transiently cotransfected with the reporter plasmids pNaCh (+RE) or pNaCh (−RE) and an internal control plasmid, pCMV-βgal, which contains the β-galactosidase reporter gene under the control of cytomegalovirus promoter. The average CAT values, normalized to β-galactosidase activity, from three experiments were denoted as a percentage of pNaCh (−RE) promoter activity. As shown in FIG. 4A, whereas PC12 cells did not show REST/NRSF activity, HeLa cells did, as expected. Under these conditions, C2C12 cells showed high endogenous REST/NRSF activity.

To determine if transient expression of REST-VP16 in C2C12 cells could counter endogenous REST/NRSF-dependent repression and cause stimulation of the RE-dependent promoter activity, we carried out the transcriptional assays using pNaCh (+RE) and pNaCh (−RE) in these cells as described above. Vector DNA and plasmid DNA encoding REST/NRSF or Gal4-VP16 were used as control DNA. As shown in FIG. 4B, none of the plasmids affected the NaCh (−RE) promoter activity. In contrast, the NaCh (+RE) promoter activity, as compared to NaCh (−RE), was repressed in the presence of the vector, pREST, and pGal4-VP16 plasmids, indicating endogenous REST/NRSF activity. On the other hand, pREST-VP16 could stimulate NaCh (+RE) promoter activity, indicating that REST-VP16 can functionally conter the endogenous REST/NRSF-dependent repression and stimulate the RE-dependent promoters in C2C12 cells, as was seen in other cell types (Immaneni et al., 2000; Lawinger et al., 2000).

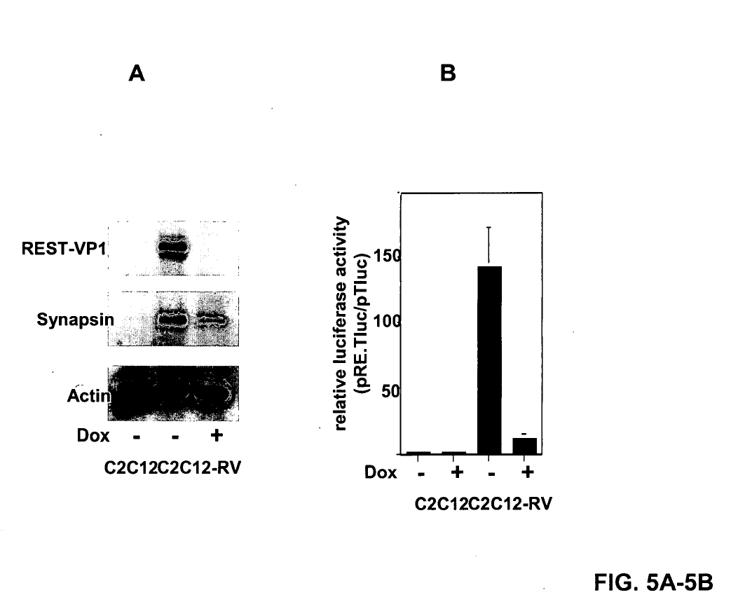

Stable REST-VP16 expression in proliferating myoblasts causes activation of neuronal differentiation genes. To investigate the role of long-term expression of REST-VP16 in myoblasts, C2C12 clonal cells were generated by using the bidirectional doxycycline-regulated vector pBig2r (Strathdee et al., 1999). The clonal cell line C2C12-RV stably expressed the REST-VP16 protein when grown without doxycycline (−Dox) for 2 days, as shown by western blotting (FIG. 5A). In contrast, C2C12 (−Dox) cells and C2C12-RV cells grown with doxycycline (+Dox) did not produce detectable levels of REST-VP16 protein.

To determine whether the REST-VP16 protein was transcriptionally active in the clonal cells, a reporter plasmid system was used (Immaneni et al., 2000; Lawinger et al., 2000). Plasmids containing a luciferase reporter gene were transfected under the control of a TATA box alone (pT.luc) or a TATA box plus two copies of the wild-type RE1/NRSE sequence (pRE.T.luc) into C2C12 and C2C12-RV (+ and −Dox) cells. These studies showed that C2C12-RV (−Dox) cells activated the REST-dependent basal promoter 145-fold more strongly than C2C12 cells (+ or −Dox) did and 20-fold more strongly than C2C12-RV (+Dox) cells (FIG. 5B). Thus, C2C12-RV (−Dox) cells produced high levels of transcriptionally functional REST-VP16 protein.

To determine whether the REST-VP16 protein produced in the clonal cells activated its cellular target, the terminal neuronal differentiation genes, were assayed using Western blot with an anti-synapsin antibody (FIG. 5A). As shown in FIG. 5A, strong synapsin expression was detected only in REST-VP16-expressing cells, indicating that REST-VP16 activated its cellular target genes in C2C12-RV (−Dox) cells. Interestingly, a low level of synapsin expression was also detected in C2C12-RV (+Dox) cells, probably as a result of the leakiness of the doxycycline-regulated system.

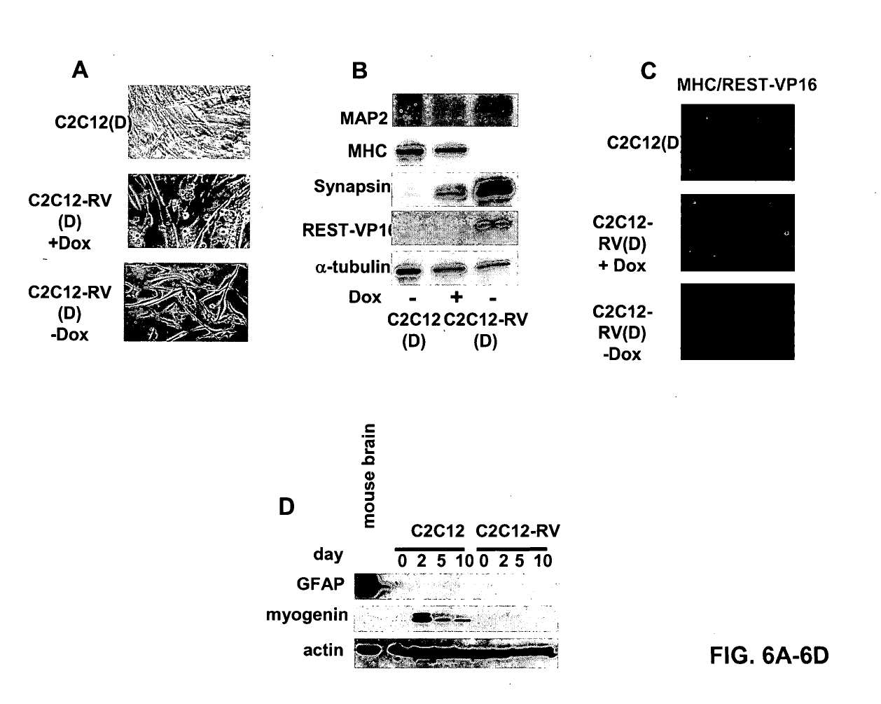

REST-VP16 expression in myoblasts growing under muscle differentiation conditions blocks muscle differentiation and produces neuronal phenotype. To determine the effect of the expression of REST-VP16 in C2C12 cells grown under muscle-differentiation conditions, C2C12 and C2C12-RV cells were grown with low concentrations of growth factors C2C12(D) and C2C12-RV(D) (+ and −Dox) for 6 days. A light-microscopic examination revealed that in the absence of REST-VP16 expression in C2C 12(D) or C2C 12-RV(D) (+Dox) cells, the cells formed multinucleated muscle fibers (FIG. 6A), as expected. In contrast, cells expressing REST-VP16 (C2C12-RV (−Dox) cells) did not form muscles. Instead, they formed mononucleated cells, some of which had long, neurite-like structures. To further characterize these cells, western blot analysis for muscle and neuronal proteins was performed. As shown in FIG. 6B, REST-VP16 was not detected in C2C12(D) cells and C2C12-RV(D)(+Dox) cells, and the myosin heavy chain (MHC) gene, a marker for differentiated muscles, was detected. Expression of synapsin, a target of REST-VP16, was not detected in C2C12(D) cells. A low level of synapsin expression was detected in C2C12-RV(D) (+Dox) cells, presumably because of the leakiness of the doxycycline system, similar to that seen earlier (FIG. 5A). In contrast, C2C12-RV(D) (−Dox) cells produced the REST-VP16 protein and a high level of synapsin but did not express MHC. Interestingly, C2C12-RV(D) (−Dox) cells also produced MAP2, indicating that expression of REST/NRSF-regulated neuronal differentiation genes through REST-VP16 also triggers the activation of other neuronal differentiation genes that are not direct targets of REST/NRSF. This correlation between REST-VP 16 and MAP2 expressions was observed in almost all cells.

To confirm the inverse correlation between REST-VP16 expression and MHC expression, C2C12(D), C2C12-RV(D) (+Dox), and C2C12-RV(D) (−Dox) cells were subjected to double immunofluorescence imaging with anti-VP16 and anti-MHC antibodies. Expression of MHC, but not REST-VP16, was observed in C2C12(D) cells (FIG. 6C). In contrast, C2C12-RV(D) (−Dox) cells did not express MHC but did express REST-VP16. The leaky growth conditions allowed some C2C12-RV(D) (+Dox) cells to express REST-VP16 and some to express MHC. However, the cells that expressed REST-VP16 did not express MHC, and the cells that expressed MHC did not express REST-VP16. Thus, these studies showed that expression of REST-VP16 in C2C12 cells under muscle differentiation conditions blocked muscle formation and MHC gene expression and caused the expression of neuronal terminal differentiation marker genes, some of which are targets of REST/NRSF and others not.

The differentiation of C2C12 cells into muscle follows a very well-defined pathway, expressing several muscle differentiation factors in a sequential fashion (Walsh and Perlman, 1997; Odelberg et al., 2000; McGann et al., 2001; Shen et al., 2003). Myogenin is one such factor that appears at the onset of muscle differentiation in C2C12 cells around two days in muscle differentiation medium (Shen et al., 2003). To examine whether REST-VP16-mediated entry of C2C12-RV cells to the neuronal pathway occurred before they entered the muscle differentiation pathway, or whether C2C12-RV cells had to wait until they entered the muscle differentiation pathway, a western blot was performed with an anti-myogenin antibody (FIG. 6D). The level of actin expression was used as an internal control, as before. As shown in FIG. 6D, the onset of myogenin expression appeared in C2C12 cells at about day 2 of muscle differentiation and then decreased with further differentiation, as expected (Shen et al., 2003). In contrast, C2C12-RV cells did not show myogenin expression during any time of their differentiation, indicating that REST-VP16 expression in C2C12-RV cells growing under muscle differentiation conditions immediately turned them towards the neuronal pathway without entering the muscle differentiation pathway.

To confirm that C2C12-RV(D) cells did not express glial markers, such as glial fibrillary acidic protein (GFAP), cell extracts prepared from C2C12 and C2C12-RV cells at various days in differentiation medium were subjected to western blotting analysis with anti-GFAP antibody. Mouse brain extract was used as a positive control for the expression of GFAP and anti-actin antibody was used as an internal control. As shown in FIG. 6D, GFAP was not expressed in any of these cells, indicating that REST-VP16 expression in C2C12 cells converted them only into neuronal phenotype.

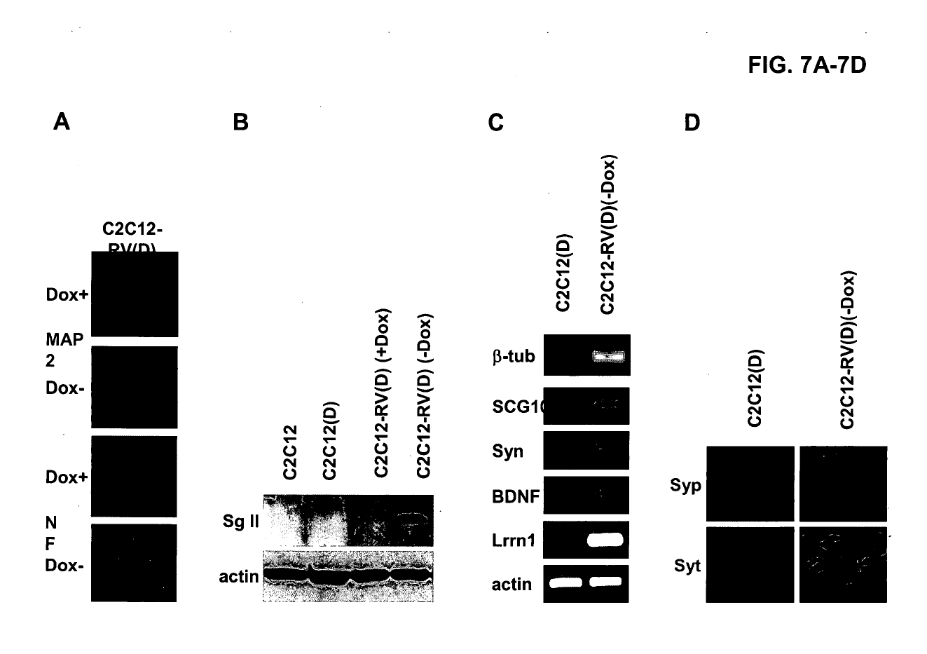

Stable REST-VP16 expression in myoblasts enables them to survive mitotic inhibitors and exhibit neuronal phenotype. To examine whether C2C12-RV(D) (−Dox) cells that are grown under muscle differentiation conditions can survive in the presence of mitotic inhibitors, a property of nondividing neurons, C2C12-RV(D) (−Dox) cells were further cultured for 14 days in the presence of mitotic inhibitors. The cells were then examined by light and immunofluorescence microscopy with anti-MAP2 and anti-neurofilament antibodies. As shown in FIG. 7A, most of the C2C12-RV(D) (−Dox) cells, unlike the C2C12 cells, survived the mitotic inhibitors, expressed the MAP2 and neurofilament proteins.

To determine the changes in the global gene expression patterns of the various cells types, gene expression profiling of C2C12, C2C12(D), and C2C12-RV(D) (−Dox) cells were performed using Affymetrix mouse chips. A large number of genes were differentially expressed among the three cell types. The genes most differentially expressed in the C2C12-RV(D) (neurons) cells compared with the C2C12(D) (muscle) cells are shown in Table 2. Likewise, the genes most differentially expressed in C2C12(D) cells, in comparison to C2C12-RV(D) cells are shown in Table 3. The C2C12(D) cells expressed mostly muscle dfifferentiation genes, as expected, and the C2C12-RV(D) cells expressed a large number of neuronal differentiation genes (Schoenherr et al., 1996; Lunyak et al., 2002). These genes included widely used neuronal markers, such as MAP2 and neuronal β-tubulin, but also neuronal receptors, such as glycine receptor, glutamate receptor, adrenergic receptor, cholinergic receptor, etc. They also included neuronal channels, such as voltage-gated calcium channel, calcium activated chloride channel, voltage-gated potassium channel, voltage-gated sodium channel, etc. Among the topmost upregulated genes in the C2C12-RV(D) cells were the genes that code for components of neuroendocrine secretory vesicles (Tooze et al., 2001), such as secretogranin II, chromogranin A, and brain-specific Purkinje cell protein 4. Western blot analysis with an anti-secretogranin II antibody confirmed that secretogranin II was expressed only in the C2C12-RV(D) (−Dox) cells, not in the C2C12 (−Dox), C2C12(D) (−Dox), or C2C12-RV(D) (+Dox) cells (FIG. 7B).

To further characterize the neuronal properties of C2C12-RV(D) (−Dox) cells, RT-PCR assays were used to determine expression of several neuronal genes in C2C12(D) and C2C12-RV(D) (−Dox) cells. As shown in FIG. 7C, several well-characterized neuronal REST/NRSF-target genes, such as neuronal β-tubulin, SCG10, synapsin, and BDNF were upregulated in C2C12-RV(D) (−Dox) cells compared to C2C12(D) cells. Interestingly, C2C12-RV(D) (−Dox) cells but not C2C12(D) cells also expressed leucine-rich repeat protein 1, neuronal (Lrm 1), a neuronal gene that is not known to be a direct target of REST/NRSF but was observed in the Affymetrix microarray data.

To further characterize the neuronal properties of C2C12-RV(D) (−Dox) cells by immunofluorescence assay, C2C12(D) and C2C12-RV(D) (−Dox) cells were stained with anti-synaptophysin and anti-synaptotagmin antibodies. Synaptophysin is a known target of REST/NRSF (Schoenherr et al., 1996) and synaptotagmin was found to be upregulated in C2C12-RV(D) cells in an Affymetrix microarray. As shown in FIG. 7D, C2C12-RV(D) but not C2C12(D) cells expressed these neuronal markers both in the cell body and in neurites, as expected (Dandoy-Dron et al., 2003; Ogoshi and Weiss, 2003). Thus, these experiments indicated that there was a global change in the gene expression profiles of these cell types and that REST-VP16-expressing myoblasts began expressing a large number of neuronal genes upon differentiation.

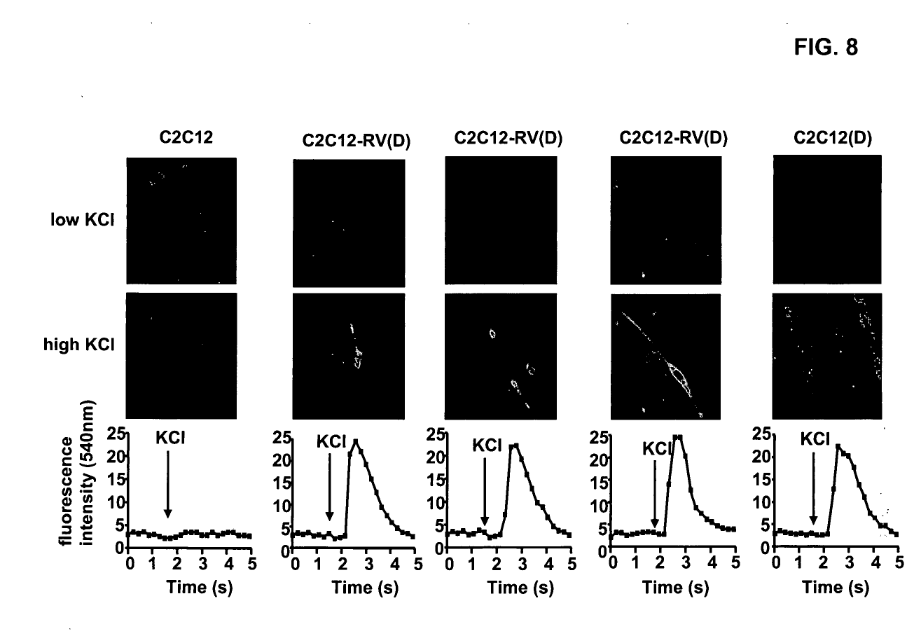

REST-VP16 expression in myoblasts produces neurons with physiological activities. Depolarization-dependent calcium influx is an intrinsic property of synaptic vesicles, occurs in both mature and developing neuronal processes, and is independent of the presence of synaptic contacts (Bischofberger and Schild, 1995; Matteoli et al., 1995; Pincus et al., 1998; Roy et al., 2000; Shen et al., 2003). This influx also occurs in functional muscle fibers (Chawla et al., 2001). To determine whether C2C12-RV(D) (−Dox) cells also undergo depolarization-dependent calcium influx, intracellular Ca++ levels were measured by loading live C2C12, C2C12(D), and C2C12-RV(D) cells with the fluorescent calcium indicator Fluo-3 in the absence of Dox and examined them with a confocal laser system. Fluo-3 is practically nonfluorescent unless bound to Ca++. These cells were then depolarized with high K⁺, and the fluorscence was measured every 0.25 sec for a total of 5 sec. As shown in FIG. 8, there was a rapid, reversible calcium influx in response to K⁺ in C2C12(D) and C2C12-RV(D) cells but not C2C12 cells. Thus, both C2C12(D) and C2C12-RV(D) cells showed physiological activity, namely, depolarization-dependent calcium influx. C2C12(D) and C2C12-RV(D) cells were cultured under similar conditions, which suggests that the neuronal properties of C2C12-RV(D) cells are not due to the differences in culture conditions but are inherent properties of these cells.

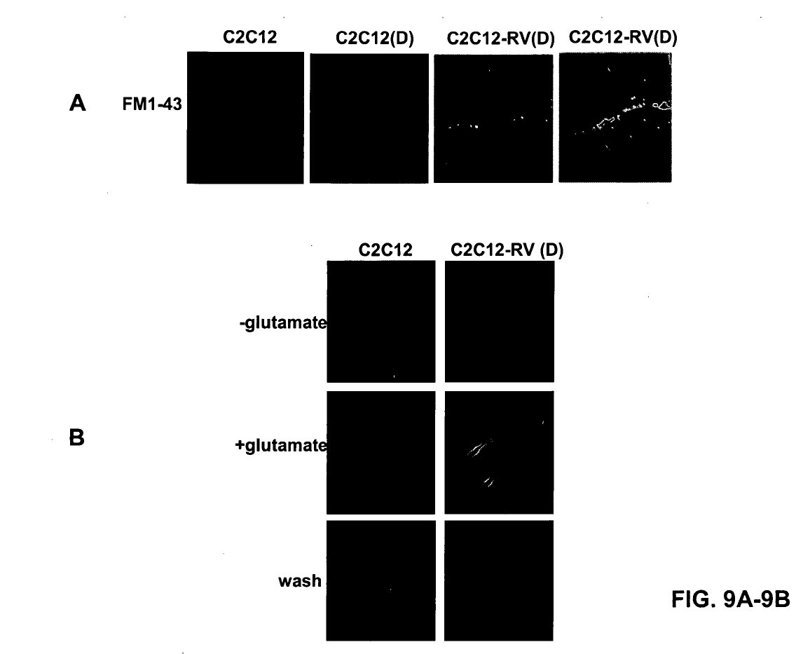

Because depolarization-dependent calcium influx is a property of both neurons and muscles, other physiological properties were examined that are specific for neurons. Synaptic vesicle recycling is a property of living nerve terminals, and the fluorescent dye FM1-43 is frequently used to label nerve terminals undergoing synaptic vesicle recycling (Betz and Bewick, 1992). The dye, which is fluorescent upon excitation at 470 nm, can be internalized from the culture medium during synaptic vesicle cycling with high K+. It does not transfer from internalized vesicles to an endosome-like compartment during the recycling process, and therefore it is possible to measure fluorescence (Murthy and Stevens, 1998). Furthermore, each fluorescent spot is believed to be a cluster of hundreds of synaptic vesicles (Betz and Bewick, 1992). This is an inherent property of neurons and is not observed in muscles. To determine whether C2C12(D) and C2C12-RV(D) cells, both of which underwent depolarization-dependent calcium influx, also experienced synaptic vesicle recycling, these cells were loaded with FM1-43 in high-KCl buffer and then washed to remove excess dye on the plasma membrane. The internalization of FM1-43 was then detected by excitation at 470 nm and the fluorescent spots were then examined by fluorescence microscopy. Fluorescent spots were observed in the neurites of C2C12 RV(D) cells but not in C2C12(D) cells or C2C12 myoblasts (FIG. 9A), indicating that C2C12-RV(D) cells have the properties of functional neuron-specific synapses.

Glutamate-induced calcium influx is another intrinsic property of neurons. To determine whether C2C12-RV(D) cells have this property, the cells' intracellular Ca++ levels were measured with and without glutamate using the fluorescent calcium indicator Fluo-3 as described above. As shown in FIG. 9B, C2C12-RV(D) cells but not C2C12 cells underwent a rapid increase in intracellular Ca++ in the presence of glutamate as evidenced by the fluorescent signal, which returned to baseline upon wash. Thus, taken together, the above experiments showed that C2C12-RV(D) cells exhibit physiological properties of neurons.

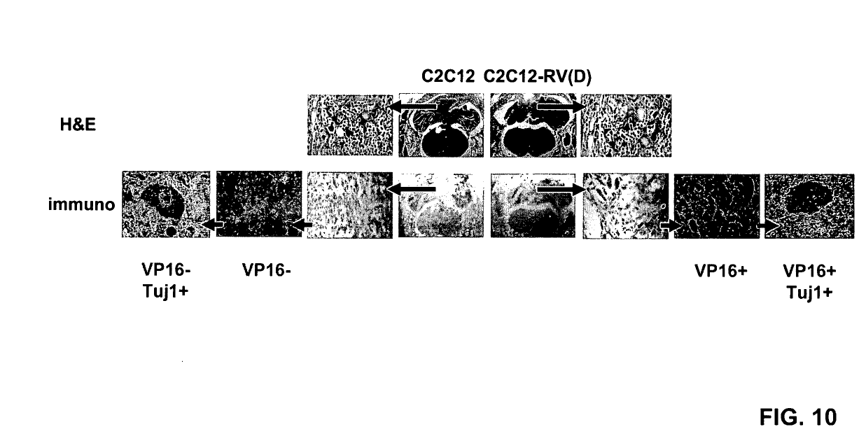

In vitro differentiated neurons produced by REST-VP16-expressing myoblasts incorporate into adult brain without forming tumors. In vitro differentiated neurons produced by REST-VP16-expressing myoblasts were tested to see if the cells could survive in the brain and whether they formed tumors, C2C12 cells and in vitro-differentiated C2C12-RV(D) cells were injected into the cerebella of mice. These studies with differentiated neurons were performed to exclude the possibility of cell fusion in the brain as a mechanism of neuron formation. Mice were sacrificed 8 weeks later, and examined the status of the injected cells in paraffin sections of the brains by hematoxylin-eosin and immunohistochemical staining with anti-VP16 antibodies. As a control, a neuronal tumor (meduloblastoma) cell line was used. However, the latter sets of mice were sacrificed after 4 weeks as they showed signs of morbidity by that time. Whereas the medulloblastoma tumor cell line formed a large tumor within 4 weeks, neither C2C12 nor C2C12-RV(D) cells formed tumors after 8 weeks. As shown in FIG. 10, C2C12-RV(D) cells, which are tagged with VP16 protein, survived in the brain as detected by the presence of signal with anti-VP16 immunohistochemistry and appeared to be distributed in the brain structures (the magnified view of C2C12-RV(D) cells show nuclear localization of VP16 signal). To determine whether the VP16-positive C2C12-RV(D) cells still expressed neuronal differentiation markers, double-immunostaining assay with the anti-neuronal β-tubulin antibody (Tujl) was used. As shown in FIG. 10, whereas the endogenous neuronal cells expressed only neuronal β-tubulin and not VP16, as expected, C2C12-RV(D) cells expressed both VP16 (nuclear) and neuronal β-tubulin (cytoplasmic), indiacting that the neuronal cells can survive in the brain and continue to express neuronal markers.

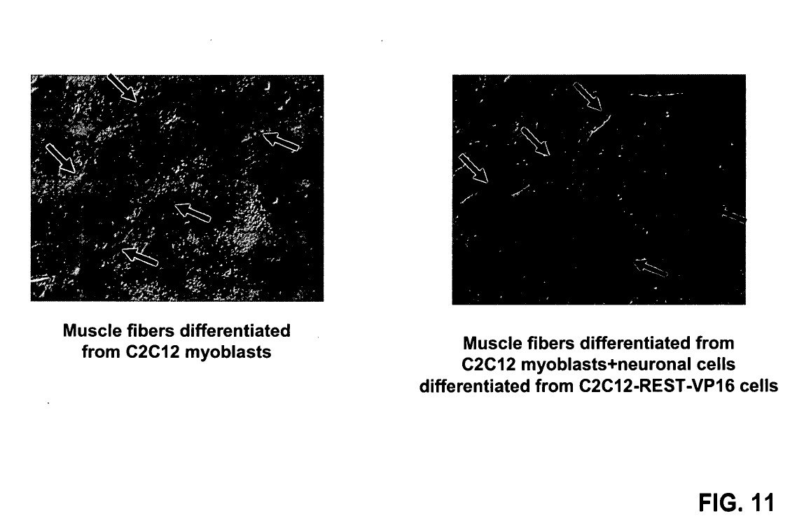

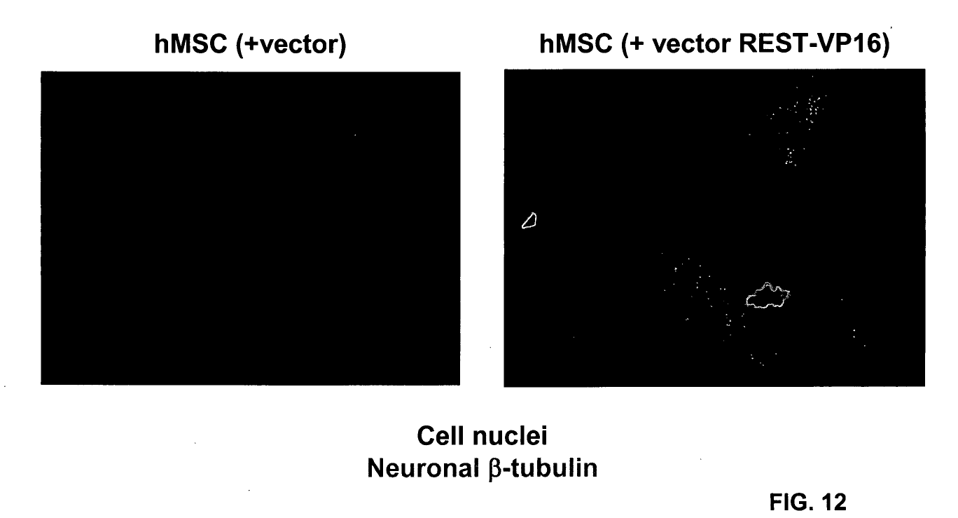

REST-VP16-mediated, Myoblast-derived neuronal cells show functional neuromuscular junction. To determine whether the REST-VP16-mediated, myoblast-derived neuronal cells could produce functional neuromuscular junction, the differentiated C2C12-REST-VP16 neuronal cells were cocultured with differentiated C2C12 muscle fibers. The presence of functional neuromuscular junctions on muscle cells were assayed by acetylcholine receptor (AchR) aggregation and subsequent detection by alexa647 conjugated-bungarotoxin staining. As shown in FIG. 11, AchR aggregation was not observed in C2C12 muscle fibers, but was observed on C2C12 muscle fibers only when they were cocultured with the neuronal cells (FIG. 12), indicating that REST-VP16-mediated, myoblast-derived neuronal cells were capable of producing functional neuromuscular junction (arrows show muscle fiber boundary).

REST-VP16 causes neuronal phenotype in human mesenchymal stem cells (hMSC). To determine whether REST-VP16 could also cause neuronal phenotype in hMSCs, the cells were transfected with pcDNA 3.1 vector DNA (control) or pcDNA 3.1 vector DNA containing REST-VP16, and subjected to immunocytochemistry to detect the production of neuronal differentiation marker, neuronal α-tubulin, using Tuj-1 antibody. As shown in FIG. 12, neuronal α-tubulin (green) was seen in hMSCs only when they were transfected with pcDNA 3.1 vector DNA containing REST-VP16. These data indicate that REST-VP16 is capable of producing neuronal phenotype in hMSCs.

### EXAMPLE 2

### Activation of REST/Nrsf Target Genes 1N Neural Stem Cells is Sufficient to Cause Neuronal Differentiation

A. Materials and Methods

Plasmids. The Nhe I/Xho I fragment of pcDNA3.1-REST-VP16 (7) was subcloned into the Nhe I/Xho I digested plasmid pBig2r (Strathdee et al., 1999). The clone obtained was confirmed by sequencing the junction region. Construction of pNaCh, pNaChΔRE1, pTLuc, pRE.Tluc, pREST/NRSF, pGal4-VP16, and pREST-VP16 has been described elsewhere (Immaneni et al., 2000; Lawinger et al., 1999; Watanabe et al., 2004). The pCMV-PGAL was purchased from Stratagene Co.

Cell culture and differentiation. Mouse multipotent neural stem cells C17.2 (NSCs) were originally described by Snyder et al. (1992) and were used throughout this study. The NSCs were maintained in Dulbecco's modified Eagle's medium (DMEM) supplemented with 10% fetal calf serum (FBS) (GIBCO) and 5% horse serum (GIBCO) (growth medium) and never grown to confluence. For REST-VP16-mediated differentiation, cells were grown on poly-L-lysine (PLL) (Sigma)-coated CC2 (Lab-Tek) chamber slides for 2 days, 8 days, or 16 days in the medium described above. For retinoic acid and mitotic inhibitor treatments, cells were grown in growth medium for 24 hours on 10 μg/ml PLL-coated Corning dishes for 24 hours; the medium was then changed to growth medium with 10 μm retinoic acid (Sigma) for 4 weeks to induce neuronal differentiation. Cells were split 1:4 by trypsinization and subcultured on PLL and laminin (Sigma)-coated Corning dishes for 2 days. Cells were then treated with DMEM containing 5% FBS and the mitotic inhibitors 10 μM 5-fluoro-2′-deoxyuridine, 10 μM uridine, and 1 μM cytosine β-d-arabinofuranoside for 5 days. All media were changed every 2 days.

Poly-L-lysine and laminin coating. Coating of Corning culture dishes or CC2 chamber slides with PLL or PLL plus laminin used 10 μg/ml PLL in distilled water. This solution was applied for 15 min, aspirated, washed five times for 10 min each, aspirated, and air-dried. For laminin coating of PLL-coated Corning dishes or CC2 slides, 15 μg/ml laminin in phosphate-buffered saline containing 1 mM CaCl₂ and 1 mM MgCl₂ at room temperature was applied, and the dishes or slides were and incubated overnight. Excess laminin solution was aspirated from the dishes or slides, and the cells were rinsed twice with culture medium, air-dried, and used immediately.

Stable transfection. C17.2 cells were transfected with pBig2r or pBig2r-REST-VP16 plasmids using Fugene 6 (Roche) according to the manufacturer's instructions. Two days after transfection, 750 μg/ml Hygromycin B (Roche) was added for clone selection. The culture medium was refreshed every 2 days with 4 μg/ml doxycycline (Sigma). Doxycycline-inducible clones, pBig2r-REST-vp16 and vector pBig2r, were selected by induction of REST-VP16 and tTA after removal of doxycycline for 2 days and identified by western blot analysis. After selection, clones were maintained in medium containing 500 μg/ml Hygromycin B. During the assays, the concentration of Hygromycin B was reduced to 250 μg/ml to avoid removal of the transgene.

Calcium imaging. Living-cell microscopy to detect depolarization-dependent calcium influx was performed as previously described (Pincus et al., 1998; Roy et al., 2000; Watanabe et al., 2004). A confocal laser system (Olympus Fluoview FV500) and the fluorescent calcium indicator Fluo-3 (Molecular Probes) were used to measure the intracellular free calcium concentration. Cells were loaded with Fluo-3 and depolarized with high-KCl (100 mM) Ringer solution as previously described (Bischofberger and Schild, 1995). Fluorescence was measured every 0.25 s for a total of 12 s. For the glutamate experiment, 10 μM glutamate was included in Ringer solution.

SDS-polyacrylamide gel electrophoresis and western blot. Cells were cultured on six-well Corning plates for 2 days or on PLL coated six-well Corning plates for 8 days without doxycycline. Cells were then rinsed and lysed directly in laemmli sample buffer (Bio-Rad). Lysates were vortexed vigorously, boiled for 5 min, and electrophoresed on SDS-PAGE-ready gels (Bio-Rad). Gels were transferred to Hybond-P PVDF membranes (Amersham Biosciences, Buckinghamshire, UK), and blots were detected using ECL-plus reagents (Amersham).

Antibodies. The following antibodies were used: anti-VP16 (1:100) (Clontech), anti-unique β-tubulin (western blot 1:500, immunofluorescence 1:1000) (Tujl; Covance Research Products), anti-synaptotagmin I (1:500) (AB5600; Chemicon), anti-MAP2 (1;1000) (HM-2; Sigma), anti-α-tubulin (1:1000) (B-5-1-2; Sigma), anti-actin (1:2000) (1-19; Santa Cruz), horseradish peroxidase-conjugated anti-mouse, anti-rabbit IgG (H+L) (1:20,000) (Amersham) and Cy3-labeled anti-mouse or anti-rabbit IgG (H+L) (1; 1000) (Amersham).

Reporter gene assays. C17.2 cells and c17.2-pBig2r and C17.2-REST-VP16 clones were plated in six-well Corning plates for 3 days in DMEM complete medium without doxycycline. pT-Luc or pRE-T-Luc (2 μg) was transfected by Fugene 6 (Roche) according to the manufacturer's instructions. Cells were collected after 48 h and assayed for protein concentration using the BCA protein assay kit (Pierce) and for luciferase activity as previously described (Immaneni et al., 2000).

Immunofluorescence. Cells were fixed on PLL-coated or PLL plus laminin coated CC2 chamber slides (see Cell culture and differentiation) for 20-30 min at room temperature with either 4% paraformaldehyde in PBS or 10% buffered formalin, rinsed in PBS, permeabilized, and blocked in blocking buffer (5% nonfat dry milk (Nestle)-0.3% TritonX-100 (Sigma)-PBS) for 30 min at room temperature. Primary antibodies were diluted in the blocking buffer and incubated overnight at 4° C. under humidified conditions. Secondary antibodies were diluted in PBS and incubated for 1 h at room temperature. For nuclear staining, 0.1 μg/ml Hoechst 33328 (Molecular Probes) in water was used. Photographs were taken with a Hamamatsu 5880 color CCD camera, equipped with a Leica DMR microscope. Double-label images were assembled in Adobe Photoshop.

B. Results

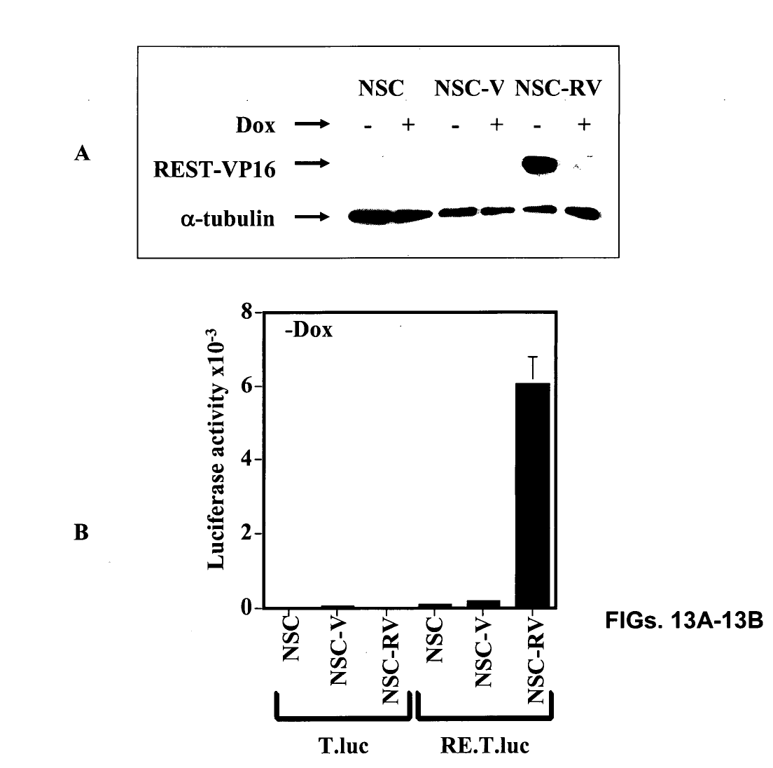

Construction of NSC clones stably expressing doxycycline-regulated REST-VP16. To determine the effect of REST-VP16 expression in NSCs, stable C17.2 mouse clonal NSCs were generated by using a bidirectional doxycycline-regulated vector, pBig2r (Strathdee et al, 1999). These NSC lines were generated by transduction of v-myc into the external granule layer cells of normal mouse cerebellum (Snyder et al., 1992). These cells can differentiate into normal neuronal and glial cells in vitro and in vivo and they do not form tumors in the brain and can be integrated into the normal brain structures, indicating that they have the functional, endogenous machinery to perform normal biological functions of NSCs (Snyder et al., 1992). The clonal cell line NSC-RVs stably expressed the REST-VP16 protein when grown without doxycycline (Dox) for 2 days, as shown by western blotting with anti-VP16 antibodies (FIG. 13A). In contrast, C17.2 cells (NSCs) or C17.2 cells expressing the vector alone (NSC-Vs) and grown with or without doxycycline (+Dox) did not produce detectable levels of the REST-VP16 protein. Similarly, NSC-RVs grown with doxycycline produced a low level of REST-VP16, probably as a result of the leakiness of the doxycycline-regulated system (Strathdee et al., 1999; Watanabe et al., 2004). In these experiments, anti-α-tubulin was used as an internal control. Taken together, these studies showed that NSC-RVs efficiently expressed REST-VP16 in the absence of doxycycline. The behavior of another REST-VP16 clone, NSC-RV′, was similar to that of NSC-RVs in all studies described (data not shown), indicating that the effect of the REST-VP16 clone was independent of the genomic integration site.

To determine whether the REST-VP16 protein was transcriptionally active, we devised a reporter gene assay system to be used before using a reporter plasmid system (Immaneni et al., 2000; Lawinger et al., 2000). Synthetic promoters containing a luciferase reporter gene under the control of a TATA box alone (pT.luc) or a TATA box plus two copies of the wild-type RE1/NRSE sequence (pRE.T.luc) were constructed. A transfection assay was performed in NSCs, NSC-Vs, and NSC-RVs using these plasmids. The plasmid pβ-gal was used as an internal control in these experiments, and the luciferase activity obtained from each experiment was divided by the corresponding β-galactosidase activity. As shown in FIG. 13B, a high level of luciferase activity was observed only from pRE.T.luc and only in NSC-RVs grown without doxycycline, indicating that NSC-RVs produced high amounts of transcriptionally active REST-VP16 protein. When cells were grown with doxycycline, little luciferase activity was observed (data not shown).

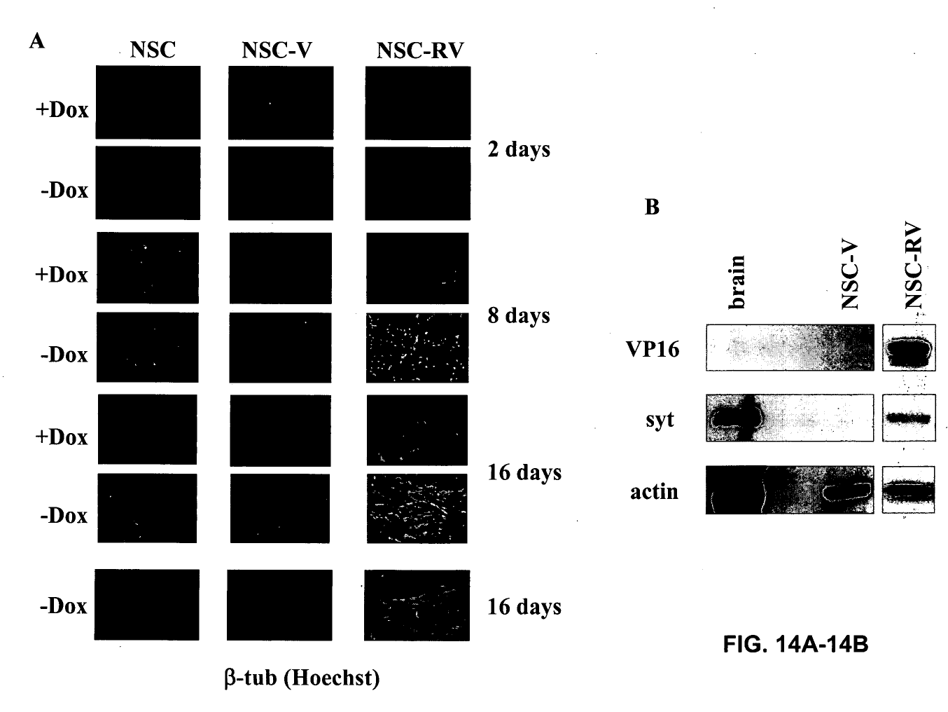

REST-VP16 converts NSCs into mature neuronal phenotype. To determine the ability of stably expressed REST-VP16 protein to induce neuronal differentiation of neural stem cells, NSCs, NSC-Vs, and NSC-RVs were allowed to grow under cycling conditions in the presence of full growth medium as described above with and without doxycycline. As shown in FIG. 14A, by day 8 NSC-RVs grown without doxycycline expressed neuronal β-tubulin, a neuronal differentiation marker, as assayed by the anti-TuJ-1 antibody, but control cells did not. This indicated that the REST-VP16 protein produced in these cells could activate its cellular target genes. By day 16, REST-VP16-expressing NSCs were highly differentiated and expressed markedly high levels of neuronal β-tubulin than the control cells. A low level of neuronal β-tubulin expression was detected in NSC-RVs grown with doxycycline, presumably because of the leakiness of the doxycycline-regulated system, similar to what was observed previously (Strathdee et al., 1999; Watanabe et al., 2004). The magnified panel in FIG. 14A shows characteristic neurite-like structures in these cells. To confirm the differentiation of NSC-RVs, we performed a Western blot analysis of 16-day-differentiated NSC-V and NSC-RV cell extracts with antibodies against another neuronal differentiation marker, synaptotagmin I, a major synaptic vesicle protein; the expression of synaptotagmin I is also directly regulated by REST/NRSF (Watanabe et al., 2004). In this study, anti-actin antibody was used as the internal control and mouse brain extract was used as a positive control for synaptotagmin I expression. As shown in FIG. 14B, NSC-RVs produced the REST-VP16 and synaptotagmin I proteins, but NSC-Vs did not.

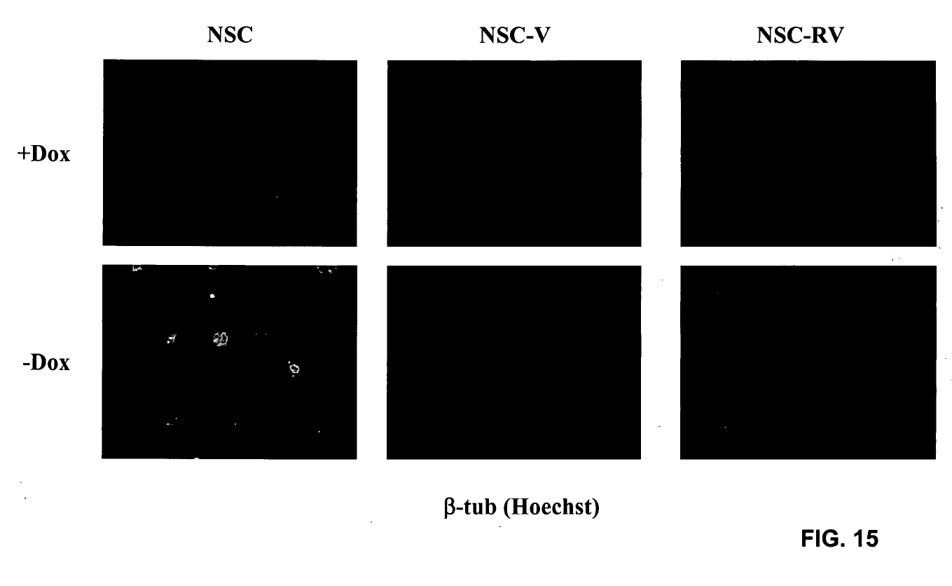

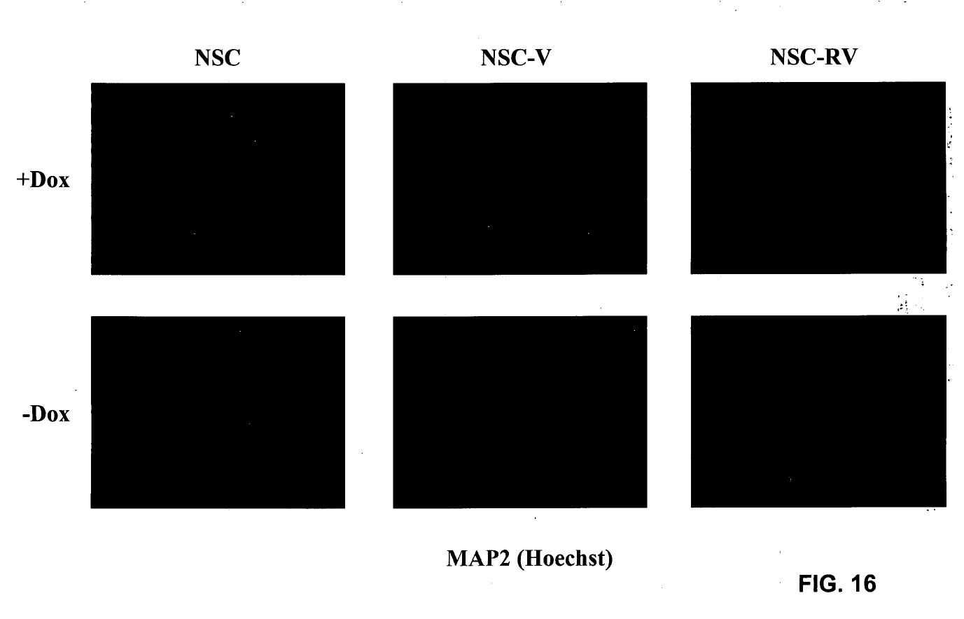

REST-VP16 causes sensitization to retinoic acid and expression of REST/NRSF-target and REST/NRSF-nontarget neuronal differentiation genes in NSCs and converts them into the neuronal phenotype. The vitamin A derivative, retinoic acid (RA), is known to be responsible for embryonic patterning and has been found to induce general neuronal differentiation as well as formation of different types of neurons in vivo and in vitro (Lawinger et al., 1999; Linville et al., 2004; Maden, 2002; Novitch et al., 2003). The inventors previously found that untreated NSCs were not sensitive to RA-dependent neuronal differentiation. The inventors sought to determine whether the expression of REST-VP16 in NSCs would produce RA sensitivity and cause them to differentiate and survive in the presence of mitotic inhibitors, a characteristic of differentiated neurons. NSCs, NSC-Vs, and NSC-RVs were grown with RA, and without and with doxycycline. The cells were then treated with the mitotic inhibitors 5-fluoro-2′-deoxyuridine, uridine, and cytosine-β-d-arabinofuranoside. The cellular nuclei were labeled with Hoechst dye, stained with antibodies for the neuronal differentiation markers neuronal β-tubulin (Tuj 1) and microtubule-associated protein 2 (MAP2), and then examined by immunofluorescence microscopy. Most of the NSC-RVs grown without doxycycline, unlike the NSCs or NSC-Vs grown with or without doxycycline, survived the mitotic inhibitors well, produced neuronal β-tubulin (FIG. 15) and MAP2 (FIG. 16) proteins, and formed neurite-like structures. Under these conditions, only a few control cells were still present, indicating that REST-VP16-expressing NSCs differentiated into neurons and that these cells survived mitotic inhibitors more efficiently than did control cells. When these experiments were repeated with antibodies against synapsin, another direct target of REST-VP16, the results were similar to those obtained for neuronal β-tubulin and MAP2 (data not shown). Interestingly, NSC-RVs produced MAP2, a neuronal differentiation marker not known to be a direct target of REST/NRSF, indicating that expression of REST/NRSF-regulated neuronal differentiation genes in NSCs through REST-VP16 also triggers the activation of other neuronal differentiation genes that are not direct targets of REST/NRSF. Taken together, these studies showed that REST-VP16 could produce neuronal differentiation of NSCs.

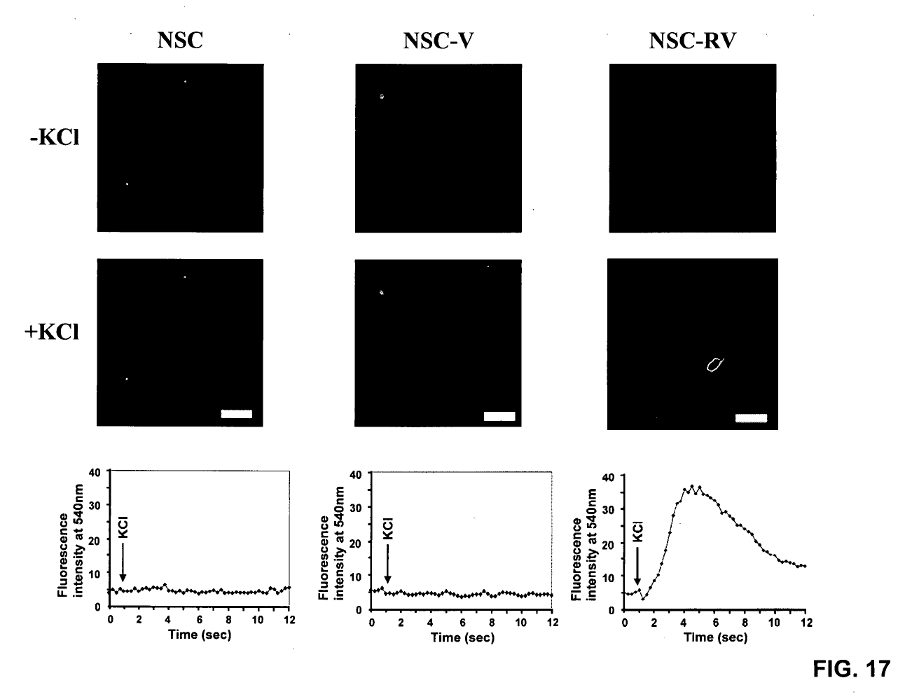

REST-VP16 expression in NSCs produces physiological activity of neurons. Depolarization-dependent calcium influx is an intrinsic property of synaptic vesicles, occuring in both mature and developing neuronal processes, and is independent of the presence of synaptic contacts (Bischofgerger and Schild, 1995; Matteoli et al., 1995; Pincus et al., 1998; Roy et al., 2000; Tooze et al., 2001). To determine whether NSC-RVs grown without doxycycline, which survived mitotic inhibitors, also undergo depolarization-dependent calcium influx, intracellular Ca++ levels were measured by loading live NSCs, NSC-Vs, and NSC-RVs with the fluorescent calcium indicator Fluo-3 in the absence of doxycycline and examined them with a confocal laser system. Fluo-3 is practically non-fluorescent unless bound to Ca++ (1995; Pincus et al., 1998; Roy et al., 2000). These cells were then depolarized with high K⁺, and the fluorescence was measured every 0.25 s for a total of 12 s. As shown in FIG. 17, a rapid, reversible calcium influx occurred specifically in response to K⁺ in NSC-RVs but not NSCs or NSC-Vs. The K+-specific signal returned to baseline upon wash (data not shown). Thus, NSC-RVs that survived mitotic inhibitors were physiologically active.

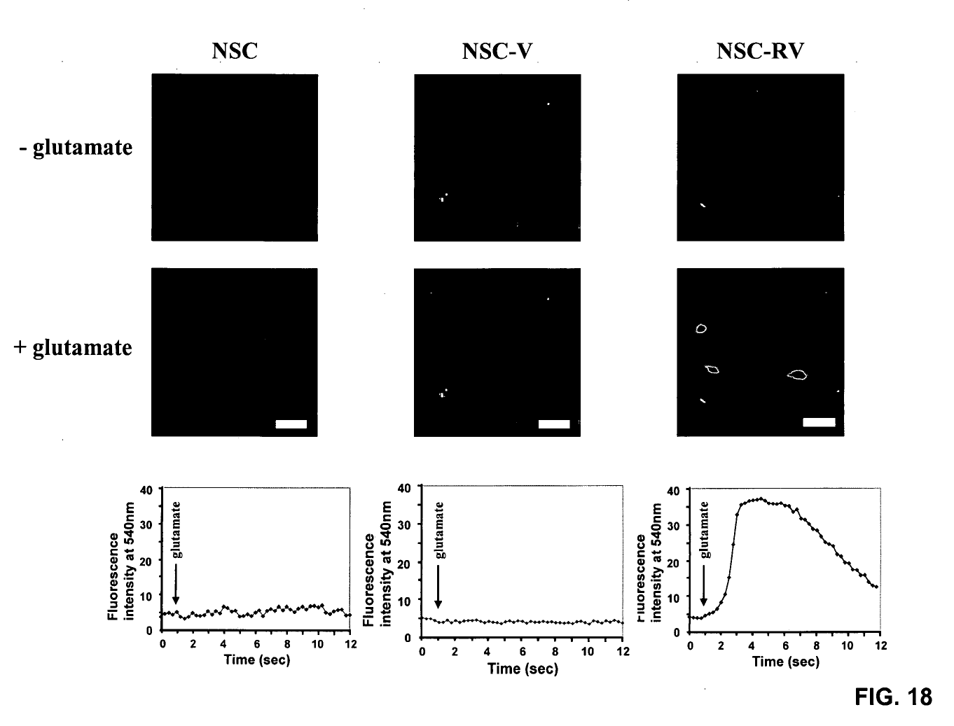

Glutamate-induced calcium influx is another intrinsic property of neurons. To determine whether NSC-RVs have this property, we measured the cells' intracellular Ca++ levels with and without glutamate. As shown in FIG. 18, NSC-RVs but not NSCs or NSC-Vs underwent a rapid increase in intracellular Ca++ in the presence of glutamate. The signal returned to baseline upon wash (data not shown). These experiments showed that NSC-RVs had the physiological properties of neurons.

### EXAMPLE 3

### Identification of Small Molecule Modulators of REST/NRSF

The transcriptional modulating compounds described in this application can be synthesized by art recognized techniques. In certain techniques, the expression of a selective marker is put under the direct control of a promoter repressed by REST/NRSF. In the presence of REST/NRSF, the selective marker is not expressed and cells survive. Inhibition of the repression may lead to expression of a cytotoxic gene, and subsequently results in cell death. Compounds that inhibit REST/NRSF are those that induce cell death under conditions of REST/NRSF expression. In other techniques, the expression of a selective marker (e.g., URA3) is put under the direct control of a promoter repressed by REST/NRSF. Under conditions of constitutive REST/NRSF synthesis the expression of URA3 is silent. Following inhibition of REST/NRSF, and in the presence of 5-FOA the synthesis of URA3 results in cell death.

A. LANCE Screening Assay for Inhibitors of REST/NRSF DNA-Binding

This example describes a method for the identification of test compounds that inhibit the interactions of purified REST/NRSF with a target DNA sequence in an in vitro system. Such molecules will potentially be able to inhibit this interaction in vivo, leading to inhibition of transcriptional repression by REST/NRSF and ultimately in differentiation of a cell into a cell exhibiting a neuronal phenotype.

1. Materials and Methods

The 6×His-tagged REST/NRSF purified according to known methods. The N-term-biotinylated double-stranded DNA has a sequence of 5′ GTGCAAAGCCATTTCAG CACCAC 3′ (SEQ ID NO:5). The antibody that may be used is the LANCE Eu-labeled anti-6×His Antibody (Eu-αHis) (Perkin Elmer cat # AD0110) which may have at least 6 Europium molecules per antibody. Streptavidin conjugated to SureLight™-Allophycocyanin (SA-APC) may be obtained from Perkin Elmer (cat # CR130-100). The Assay buffer contains 20 mM Hepes pH 7.6, 1 mM EDTA, 10 mM (NH₄)₂SO₄, and 30 mM KCl, and 0.2% Tween-20.

The plates or vials of the compounds to be tested are thawed. These stocks may be at a concentration of 10 mg/ml in DMSO or other appropriate solvent. The solutions are allowed to thaw completely, and the plates are briefly shaken on the Titermix to redissolve any precipitated compound. Thawed aliquots of REST/NRSF protein are placed on ice.

Dilutions at 1:100 of the compounds are made into a fresh 96-well polystyrene plate. The dilutions were prepared with 100% DMSO to give a final concentration of 100 μg/ml solutions. The dilutions were vortexed on a Titermix.

Fresh DTT is added to 25-50 mL of assay buffer to produce a 1 mM final concentration. Ninety μl of assay buffer is added to each of the 10 μl protein aliquots, and the solution is mixed by pipetting. These proteins are diluted to give the required amount of each of the diluted proteins, resulting in 20 μl of diluted protein per well. In preparing the solutions, 20% excess is made to allow enough for control wells. Typically, depending on the protein preps and the initial binding curves that are performed, 1000-2000 fmoles of each protein is required per well. The diluted protein solutions were the placed on ice.

Tests plates of compound can be prepared. Using a multichannel pipet, 5 μl of the compound is added to each well. 5 μl of DMSO is added to the blank and control wells, and 5 μl of the control inhibitor is added to the respective wells.

Using the multichannel pipet, 20 μl of protein is added to all wells except those designated “blank”. To these blank wells, 20 μl of assay buffer is added. The plates are covered with a plate sealer and incubated at room temperature, shaking on the Titermix, for 30 minutes.

Next, the DNA solution is prepared, with enough for at least 20% more wells than were tested. 15 μl (0.4 fmoles) is added per well. Then the DNA is diluted in assay buffer, and vortexed briefly to mix. The plate sealer is removed, and 15 μl of DNA solution is added to all of the wells. The plates are then resealed, and returned to the Titermix for a further 30 minutes.

After 25 minutes, the antibody solution is prepared. 0.4 fmoles of SA-APC and 0.125 fmoles of Eu-αHis were added per well in a total volume of 10 μl. Amounts are prepared sufficient for at least 20% excess. The plate sealer is then removed and 10 μl of antibody solution is added to every well. The plates are subsequently resealed, placed on the Titermix, and covered with aluminum foil. The plates are mixed for 1 hour. The plates were then read on the Wallac Victor V, using the LANCE 615/665 protocol.

2. Data Processing

For each plate, the mean control (i.e., signal from protein and DNA without inhibition), mean blank (background signal without protein) and mean inhibitor (P001407) LANCE665 counts are determined. The percentage inhibition by each molecule (each test well) is then determined according to the following equation: % Inhibition=100−(((test−mean blank)/(mean control−mean blank)*100). Compounds that gave 40% or greater inhibition were identified as hits and screened again for IC50.

B. Luciferase Assay

The expression of luciferase is put under the direct control of a promoter repressed by REST/NRSF in a cell constitutively expressing REST/NRSF. In the presence of an inhibitor of REST/NRSF activity, cells luminesce. The luciferase assay is used to determine if any of the compounds tested increase/reduce the luminescent signal. This indicates that the test compounds affect regulation of RE1 elements, which in turn is regulated by REST/NRSF polypeptides.

1. Materials and Methods

The bacteria that can be used are E. coli AG112 KmicF-Luc. The negative control Bacteria are E. coli AG112. The test compounds are prepared in a 10 mg/mL DMSO stock solution.

Preparation of Inoculum. Inoculum (or “Starter Inoculum”) is started the night before the day of the experiment by adding either a colony or stab of a glycerol stock to 2 mL of LB Broth. The Starter Inoculum is then placed in a 37° C. shaker incubator and allowed to grow overnight.

The following day, the Starter Inoculum is removed from the shaker and added to fresh LB Broth. For each plate to be assayed, 6 mL of LB broth is prepared, with 5-10 μL of Starter Inoculum being added per mL of added LB to form the “Test Inoculum”. In this typical example, 6 mL of LB Broth is used for each plate, or 24 mL of LB, followed by the addition of 5 μL/mL of Starter Inoculum, or 120 μL of Starter Inoculum to form the Test Inoculum.

Following preparation of the Test Inoculum, the Test Inoculum is placed in a 37° C. shaker and incubated for about 4 hours. The Test Inoculum is monitored for bacterial growth by taking OD readings at 535 nm on a spectrophotometer. The Test inoculum should be removed when the OD reaches between 0.6 and 1.5.

Preparation of Controls. Positive and negative controls are created by adding 2 uL DMSO to 198 uL LB Broth.

Preparation of Compounds. The compounds are typically screened at 25 ug/mL. Two identical plates of each compound are set up: 1 clear plate for growth (or “Clear Plate”), 1 white plate for luminescence (or “White Plate”). Next, 2 μL of each compound is taken from the daughter plate (containing 10 mg/mL stock) and added to 198 μL of LB Broth. The sample is then mixed. Next, 25 μL of the diluted test compound is added to 25 μL of LB Broth in all of the assay plates. The concentration of the compound at this stage is typically 50 μg/mL.

Preparation of Plate. 50 μL of the Test Inoculum is added to each well of the plates, except for the negative controls. Half of the negative controls receive 50 μL of AG112, while the other half of the negative controls receive 50 μL LB Broth. The final concentration of the test compound is about 251g/mL.

The Clear Plates are placed in the plate reader and read at OD535. This is the “Initial” growth read. The plates are then incubated plates for 5 hours at 37° C. After 5 hours, the plates are removed from the incubator. The Clear Plates are placed in the plate reader and read at OD535. This is the “Final” growth read.

100 μL of Promega Steady-Glo reagent is added to each well (including all controls) in the White Plates. The plates, covered with aluminum foil, are then shaken on a plate shaker set at 10000 rpm for 10 min. The plates are then placed in plate reader and read on luminescence for 1 sec per well. This is the LUMINESCENT read.

Data Analysis. To determine whether the test compound inhibited growth, the Initial growth read was subtracted from the Final growth read. This is the Subtracted Growth. The same calculation was performed for the positive controls. The results for the positive controls are typically averaged. The % Inhibition of Growth is determined using the following formula: 100-(100*Subtracted growth of sample/Average growth of Pos Controls)

To determine whether compound inhibits Luciferase, the following equation may be used: 100-(100*Luminescence for Compound/Average Luminescence of Pos Controls).

### EXAMPLE 4

### Tumor Overexpression of REST/NRSF is Countered by Rest-Transactivator Expression

A. Material and Methods

Histological and Immunohistochemical assays for medulloblastoma samples. For histological examination, surgically excised human brain tumor tissue and adjacent normal tissue samples fixed in 10% buffered formalin and embedded in paraffin were obtained from the Brain Tumor Center tissue bank at M.D. Anderson Cancer Center, stained with hematoxylin-eosine (H&E), and examined under light microscope as previously described (Lawinger et al., 2000). For immunohistochemical assays, the brain sections were stained with an anti-REST antibody (a gift of Gail Mandel) essentially as previously described (Lawinger et al., 2000). In brief, the routinely processed, formalin-fixed, paraffinembedded tissue was sectioned at a thickness of 4-5 μm. The tissue sections were deparaffinized, incubated in 0.3% hydrogen peroxide in methanol for 10 min at room temperature, washed, and treated with 1% acetic acid in phosphate-buffered saline (PBS) for 5 min. Nonspecific background binding was blocked with normal goat serum. The slides were incubated with primary anti-REST antibody overnight at 4° C. The secondary polyclonal antibody (biotinylated goat anti-rabbit antibody, diluted 1:100, Oncogene Science) was applied for 1 h at 37° C. Detection was accomplished after incubation with avidin alkaline phosphatase (Dako Corporation, Calif.), diluted 1:200, for 1 h at 37° C., followed by application of an alkaline phosphatase substrate (Gibco-BRL, Gaithersburg, Md.) and incubation in the dark for 20-30 min. The staining intensity was graded as negative (−), weakly positive (+), or strongly positive (++), and the number of tumor cells that were positively stained was expressed as a percentage of the total number of tumor cells, estimated on the basis of 15-20 representative fields from each section.

Cell culture, viruses, and mouse brain immunohistochemistry. D283 (or Daoy) medulloblastoma cells were purchased from American Type Culture Collection (Rockville, Md.) and grown as previously described (Lawinger et al., 2000). Replication-incompetent adenoviral vectors encoding the vector DNA (Ad), green fluorescent protein cDNA (Ad.GFP) or the REST-VP16 cDNA (Ad.REST-VP16) under the control of cytomegalovirus promoter/enhancer elements were generated and used to infect the Daoy cells as previously described (Lawinger et al., 2000). Deparaffinization and immunohistochemical staining were performed on a Ventana BenchMark XT Autostainer (Tuscon, Ariz.). Initially, antigen retrieval was incorporated using citrate buffer, pH 6.0, in a rice steamer, for 45 minutes, at 92 degrees centigrade. After rinsing in PBS, the sections were incubated with the primary antibody, synaptophysin (Abcam; Cambridge, Mass.; monoclonal mouse clone sy38), at the dilution of 1:50. The Avidin-Biotin-Peroxidase Conjugation method (ABC) was used by the detection kit, LSAB (Dako; Carpinteria, Calif.). The immunoreaction was visualized using 3-amino-9-ethylcarbazole (AEC) as the chromogen. Sections then were counterstained with Mayer's hematoxylin for visualization of nuclear detail.

Intracranial inoculation of cells into mice and assay of tumor formation. These studies were performed following M.D. Anderson Institutional Animal Care and Use guidelines. Daoy cells infected with Ad or Ad.REST-VP16 (2×10⁵ cells present in 5 μl cell growth medium) were inoculated in a group of 6 mice for each condition using an implantable guide-screw system described elsewhere (Lal et al., 2002). This method, which uses a guide screw, a stylet, a modified Hamilton syringe with a 26-gauge needle, and an infusion pump, allows delivery of cells to a very specific location in the brain. Several days before the inoculation, the guide screws were placed in the dorsal cranium of mice. The animals were anesthetized by intraperitoneal injection of 1.2% avertin solution made in sterile PBS per g of body weight (100% avertin solution: 10 g tribromoethylalcohol+10 ml tertiary amyl alcohol) at a dosage of 15-17 μl per g of body weight. The guide screw entry site was marked at a point 2.5 mm lateral and 1 mm anterior to the bregma, which is located directly above the caudate nucleus. A small, hand-controlled twist drill was used to make the hole, and a specially devised screwdriver was used to thread the screw into the hole and secure it. After a recovery period of 5 days after the guide screws were placed, the cells were inoculated. On the day of implantation, the cells were harvested and resuspended in Ca++- and Mg++-free PBS. The cells were kept on ice until implantation. Mice with the guide screws in place were reanesthetized as described above. The tumor cell suspension was drawn into a Hamilton syringe, which was fitted with a cuff to control the depth of injection. The needle of the Hamilton syringe was slowly lowered into the center of the guide screw until the cuff rested on the screw surface. Up to 10 mice attached to 10 syringes were positioned in a row in an Harvard Apparatus (Holliston, Mass.) infusion pump that provided continuously controlled slow injection of the cells into the caudate nucleus at a rate of 1 μl/min. After the entire volume of the cell suspension was injected, the needles were manually removed, and with a fine forcep, the stylets were positioned in the screw holes to close the system and prevent the tumor cells from leaking after inoculation. The mice were sacrificed after 4 weeks by CO₂ inhalation and their brains fixed with formalin and embedded in paraffin; 4-5 μm brain sections were examined as described above for the human medulloblastoma brain specimens. For intratumoral adenovirus injection experiments, intracranially implanted medulloblastoma cells were allowed to grow for two weeks and then each mouse was injected three times at the interval of three days with 2×10⁵ plaque-forming units of Ad.GFP or Ad.REST-VP16 present in 3 μl volume using the guide-screw system. The mice brains were then examined by magnetic resonance imaging (MRI) after an additional 4 weeks, sacrificed, and their brains were analyzed by histological methods as described above. The diameter of each tumor was determined by measuring the average of the largest and the smallest diameters and this value was used to calculate the average volume of each tumor. Because of the small number of independent samples, the p-value was calculated by the Mann-Whitney Test using SPSS statistical package (SPSS Inc., Chicago, Ill.).

Magnetic Resonance Imaging (MRI). The magnetic resonance images were acquired on a clinical 1.5T GE Signa (GE Medical Systems, Waukesha, Wis.) in the Small Animal Cancer Imaging Research Facility at The University of Texas M. D. Anderson Cancer Center (Houston, Tex., USA). Mice were anesthetized for imaging using 1.5% to 5% isoflurane inhalation anesthesia. The intracranial screws were removed from each of the mice prior to imaging. The images of the brains of the mice were acquired using a 3 cm quadrature volume resonator. Axial T1-, T2-, and post contrast T1-weighted images using Gadilinium-DTPA (Magnebist from Berlix Imaging) as a contrast agent were acquired of the brain. The axial head images covered a 4.0 cm square field-of-view. Data was acquired at 256×192 data matrices, resulting in an isotropic in plane resolution of approximately 150×200 μm. All slices were 1.5 mm thick and separated by 0.5 mm gaps. The T1-weighted images were acquired with a spin-echo pulse sequence at TE/TR=14/450 ms while the T2-weighted images were acquired with fast spin-echo pulse sequence at TE/TR=90/5000 ms with an echo train length of 12.

In situ apoptosis detection assay. Apoptosis in tumor sections was determined by using the TACS in situ apoptosis detection kit (R&D Systems, catalog #TA4625) according to the manufacturer's instructions. Formalin-fixed paraffin-embedded mouse brain sections were deparaffinized and then digested with proteinase K for permeabilization. The apoptotic DNA fragments were labeled with biotinylated nucleotides using terminal deoxynucleotidyl transferase and the biotinylated nucleotides were then detected by streptavidin-horseradish peroxidase conjugate and diaminobenzidine.

B. Results

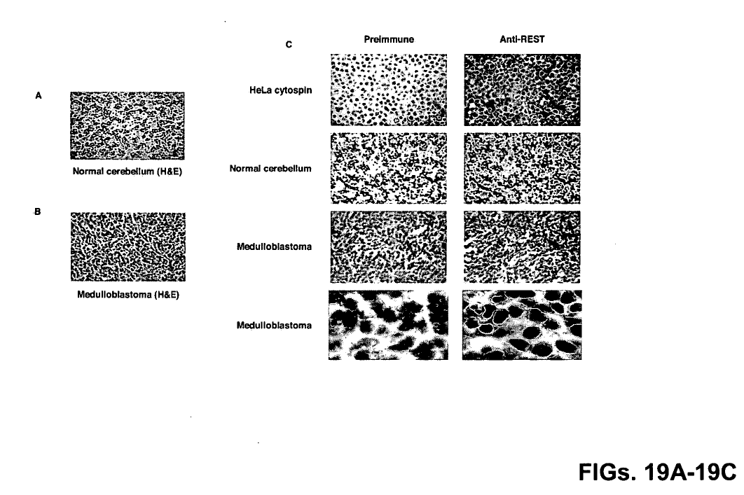

Human medulloblastoma tumors overexpress REST/NRSF. In order to determine whether human medulloblastoma tumors overexpressed REST/NRSF, 21 surgically excised, formalin-fixed, paraffin-embedded tissue specimens organized in a tissue array, were analyzed first by H&E staining (FIGS. 19A and 19B) and then by immunohistochemical analysis with an anti-REST antibody (FIG. 19C, Table 4). All specimens were obtained from the M. D. Anderson Brain Tumor Center tissue bank and were previously diagnosed by histological examination as medulloblastoma. Six normal cerebellar tissue samples adjacent to tumors, which were histopathologically distinct from the tumor tissue, were included in the array as negative controls. Medulloblastoma patient age ranged from 2 to 34 years (Table 5). The morphology shown in FIGS. 19A and 19B shows that normal cerebellar cortex tissue was comprised of granular-cell neurons with monotonously uniform, round nuclei and interspersed pale neuropil islands (glomeruli), whereas the medulloblastoma displayed disheveled, densely packed tumor cells with irregular, hyperchromatic, pleomorphic nuclei, as expected for these tumors. The immunohistochemical analysis (FIG. 19C) performed using anti-REST/NRSF antibodies revealed a strong positive signal with HeLa cells (HeLa cytospin), which express REST/NRSF, and they served as a positive control (Lawinger et al., 2000). These cells were spun down, fixed with formalin, and paraffin-embedded and processed the same way as the brain tissue. As described above, normal cerebellar tissue adjacent to the tumor was used as a negative control. While no immunoreactivity was observed in normal cerebellar tissue, REST/NRSF positivity was observed in 17 of 21 medulloblastoma tumor samples, with strong signals in 6 and weak signals in 11. The 4 specimens that did not show immunoreactivity came from patients aged 2, 8, 12, and 25 years, indicating that immunopositivity is not present in younger patients. These results indicated that REST/NRSF is a major determinant for human medulloblastoma tumors.

TABLE 4
Status of REST/NRSF immunoreactivity in human medulloblastoma
patient samples

| Patient # | Age | REST |
|---|---|---|
| 1 | 2 | − |
| 2 | 9 | + |
| 3 | 34 | + |
| 4 | 8 | − |
| 5 | 4 | + |
| 6 | 15 | ++ |
| 7 | 22 | ++ |
| 8 | 20 | + |
| 9 | 2 | + |
| 10 | 26 | + |
| 11 | 11 | + |
| 12 | 2 | + |
| 13 | 2 | ++ |
| 14 | 12 | − |
| 15 | 25 | − |
| 16 | 26 | ++ |
| 17 | 20 | ++ |
| 18 | 10 | + |
| 19 | 3 | + |
| 20 | 7 | + |
| 21 | 11 | ++ |

−, negative
+, weakly positive
++, strongly positive

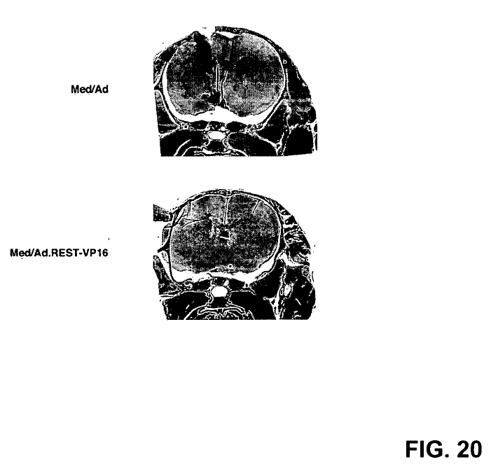

Adenovirus-mediated expression of REST-VP16 blocks the intracranial tumorigenic potential of human medulloblastoma cells in nude mice. Several medulloblastoma cell lines (D283, Daoy, and D341) overexpress REST/NRSF, and they do not express neuronal differentiation genes (Lawinger et al., 2000). These cells cells also form intracranial tumors in nude mice (Li et al., 2004). Previously, the inventors have shown that the recombinant molecule REST-VP16, which can compete with REST/NRSF for binding to RE1/NRSE and can activate neuronal differentiation genes (REST/NRSF target genes) instead of repressing them, could also block tumorigenic potential of various medulloblastoma cells at subcutaneous locations in mice (Lawinger et al., 2000). Because the intracranial environment may be critical for medulloblastoma tumorigenesis, the inventors wanted to determine whether REST-VP16 could also block the tumorigenic potential of medulloblastoma cells in orthotopic mouse models. For this purpose, D283 (or Daoy cells) cells infected with either Ad or Ad.REST-VP16 were inoculated into the brain of six 8-week-old nude mice using the implantable guide-screw system. The mice were sacrificed after 5 weeks and examined their paraffin-embedded brain sections by H&E staining (FIG. 20). Whereas no tumor was visible in any of the Ad.REST-VP16 inoculated mice tumors, large tumors appeared in 5 of the 6 Ad inoculated mice. Similar results were also obtained with Daoy medulloblastoma cells. These experiments suggest that countering REST/NRSF function and the forced expression of neuronal differentiation genes in medulloblastoma cells can interfere with the cells' tumorigenic potential in orthotopic mouse models.

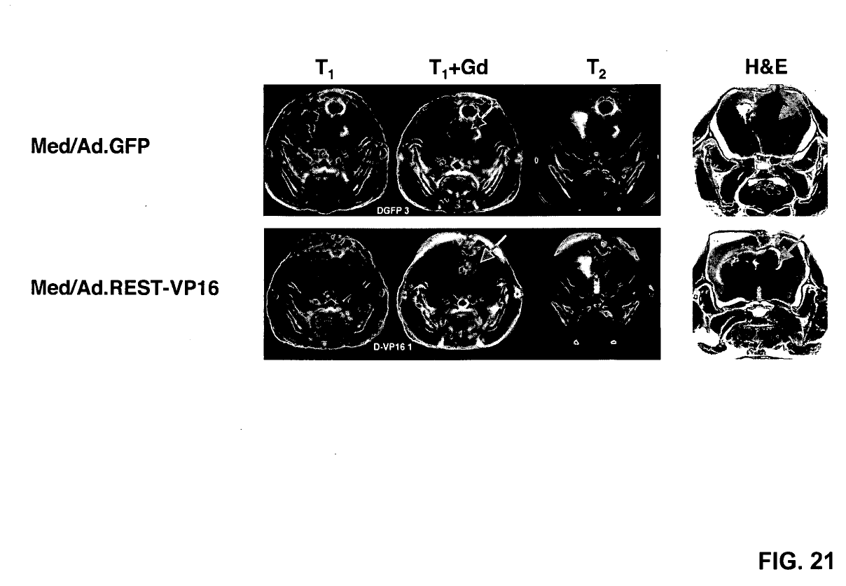

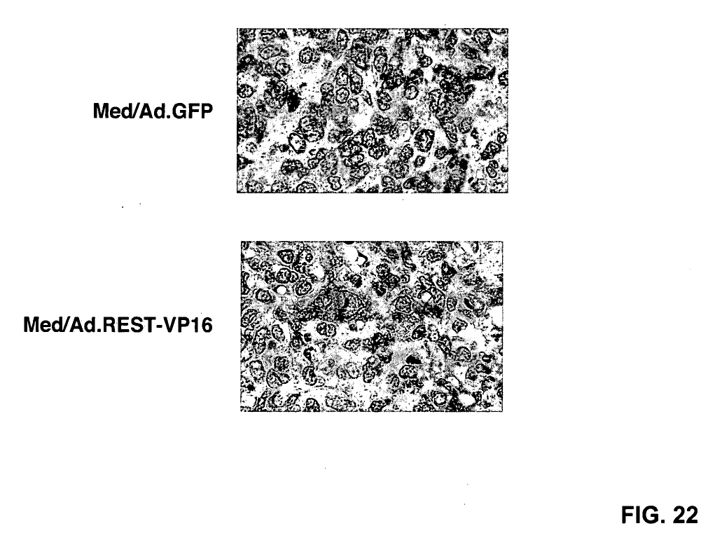

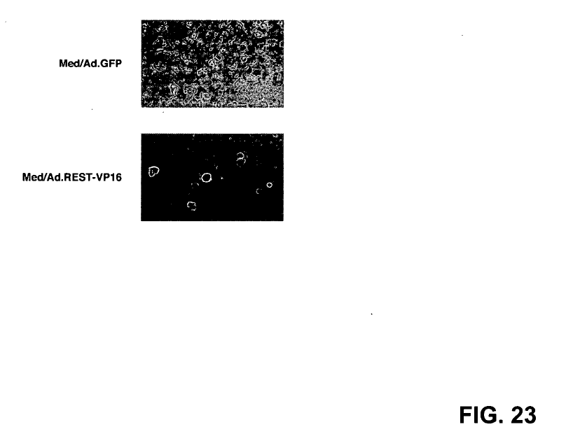

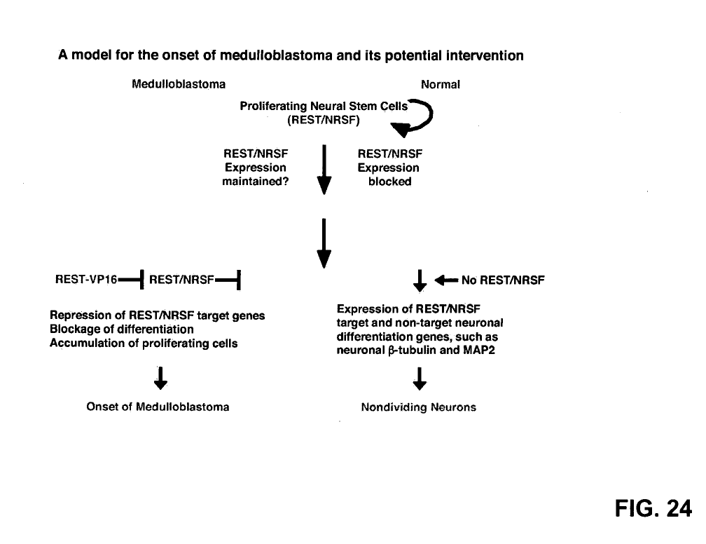

Adenovirus-mediated delivery of REST-VP16 inhibits growth of human medulloblastoma cells at intracranial locations in nude mice. To examine whether the intratumoral injection of adenoviral vectors carrying REST-VP16 would inhibit the growth of preformed medulloblastoma intracranial tumors, uninfected D283 (or Daoy cells) were injected intracranially in nude mice as described above. After 2 weeks, one group of mice was injected with Ad.GFP and the other with Ad.REST-VP16. Three injections were carried out with an interval of three days each, for a total of three injections. At the end of an additional 4 weeks period, all mice were analyzed by MRI, sacrificed and their paraffin-embedded brain sections were analyzed by H&E to detect the tumor volume (FIG. 21). The Ad-REST-VP16-injected mice showed smaller tumors with a volume of 2.10 mm³ (standard error: 0.54) as compared to mice injected with Ad.GFP that showed an average tumor volume of 13.57 mm³ (standard error: 3.07). The p-value for comparing both sets of data is 0.002 using Mann-Whitney test. Again, similar results were obtained with Daoy cells. It is interesting that in the tumorigenicity experiment described in the previous section, in which cells were directly infected with adenovirus in vitro and then inoculated into nude mice, tumors grew in none of the mice receiving Ad.REST-VP16-infected cells. In contrast, the intratumoral injection of Ad.REST-VP16 caused inhibition of tumor growth rather than regression of the tumor. This suggests that intratumoral injection delivers the virus only to a limited number of cells in the tumor, presumably causing them to undergo apoptosis (Lawinger et al., 2000). However, the spread of the virus among neighboring tumor cells was restricted because of the fact that these are replication-defective viruses. Thus, tumor cells that did not receive the virus would grow at a normal rate for the tumor. To determine whether the delivery of REST-VP16 in preformed medulloblastoma tumors indeed caused expression of terminal neuronal differentiation genes and apoptosis, the tumor sections were subjected to immunohistochemical analysis with anti-synaptophysin antibody (FIG. 22) and an in situ apoptosis detection assay that measures apoptotic cells by detecting DNA fragmentation (FIG. 23). As shown, Ad.REST-VP16, and not Ad.GFP, injected tumors showed expression of sypatophysin and distinct signs of apoptosis. A model for the onset of medulloblastoma and its potential to treatment by REST-VP16 is shown in FIG. 24.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A neuronal cell comprising a REST-transactivator.

2. The cell of claim 1, wherein the REST-transactivator comprises a Repressor Element 1 (RE1) binding domain and a transactivation domain.

3. The cell of claim 2, wherein the RE1 binding domain is a synthetic or recombinant RE1 binding domain.

4. The cell of claim 2 wherein the transactivation domain is a synthetic transactivation domain, a recombinant viral transactivation domain, or a recombinant eukaryotic transactivation domain.

5. The cell of claim 1, wherein the REST-transactivator is a polypeptide.

6. The cell of claim 5, wherein the polypepitde comprises a viral transactivation domain.

7. The cell of claim 6, wherein the viral transactivation domain is a VP16 transactivation domain.

8. The cell of claim 5, further comprising a RE1 binding domain.

9. The cell of claim 8, wherein the RE1 binding domain is a REST/NRSF DNA binding domain.

10. The cell of claim 5, wherein the REST-transactivator is encoded by an expression cassette.

11. The cell of claim 10, wherein the expression cassette is integrated into the genome of the cell.

12. The cell of claim 10, wherein the expression cassette is maintained episomally.

13. The cell of claim 1, wherein the cell is derived from a cell line.

14. The cell of claim 1, wherein the cell is derived from a primary cell.

15. The cell of claim 1, wherein the cell is derived from a stem cell.

16. The cell of claim 15, wherein the stem cell is isolated from muscle, cord blood, bone marrow, embryonic tissue or reproductive tissues.

17. The cell of claim 1, wherein the cell is derived from a progenitor cell.

18. The cell of claim 17, wherein the progenitor cell is isolated from muscle, blood, bone marrow, liver, kidney, heart, lung, brain, reproductive tissues, or skin.

19. The cell of claim 1, wherein the cell is derived from a myoblast.

20. The cell of claim 1, wherein the neuronal cell is capable of forming spheroids.

21. The cell of claim 1, wherein the neuronal is capable of forming extensions of long axons.

22. The cell of claim 1, wherein the neuronal cell is capable of forming neuronal connections with other neuronal cells in vivo.

23. A method of abrogating or reducing neuronal damage, neuronal loss or neurodegenerative disease comprising administering to a animal in need thereof a cell comprising an effective amount of a REST-transactivator.

24. The method of claim 23, wherein neuronal damage or neuronal loss is brain, muscle, or spinal cord injury or trauma.

25. The method of claim 23, wherein the REST-transactivator is administered to a cell in vivo, in vitro, or ex vivo.

26. The method of claim 23, wherein the neuronal cell is administered by implantation, injection or perfusion.

27. The method of claim 26, wherein the neuronal cell is administered by intramuscular, intradural or intracranial injection or perfusion.

28. The method of claim 23, wherein the source of the neuronal cell is the animal being treated.

29. The method of claim 23, wherein the source of the neuronal cell is a second animal.

30. The method of claim 23, wherein the animal is a human.

31. The method of claim 23, wherein the animal is diagnosed with Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum defoimans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, ophthalmoplegia, brain trauma or injury, spinal cord trauma or injury, mood disorders, or depression.


SEQUENCE LISTING (15 sequences)

SEQ ID NO: 1 - DNA, 2781 bp, Artificial Sequence
  CDS (1)..(2781)
  Description of Artificial Sequence  Synthetic Primer
atggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttgaggcatcaggtgctccagatatttacagttcaaataaagatcttccccctgaaacacctggagcggaggacaaaggcaagagctcgaagaccaaaccctttcgctgtaagccatgccaatatgaagcagaatctgaagaacagtttgtgcatcacatcagagttcacagtgctaagaaattttttgtggaagagagtgcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacaggagaacgcccatataaatgtgaactttgtccttactcaagttctcagaagactcatctaactagacatatgcgtactcattcaggtgagaagccatttaaatgtgatcagtgcagttatgtggcctctaatcaacatgaagtaacccgccatgcaagacaggttcacaatgggcctaaacctcttaattgcccacactgtgattacaaaacagcagatagaagcaacttcaaaaaacatgtagagctacatgtgaacccacggcagttcaattgccctgtatgtgactatgcagcttccaagaagtgtaatctacagtatcacttcaaatctaagcatcctacttgtcctaataaaacaatggatgtctcaaaagtgaaactaaagaaaaccaaaaaacgagaggctgacttgcctgataatattaccaatgaaaaaacagaaatagaacaaacaaaaataaaaggggatgtggctggaaagaaaaatgaaaagtccgtcaaagcagagaaaagagatgtctcaaaagagaaaaagccttctaataatgtgtcagtgatccaggtgactaccagaactcgaaaatcagtaacagaggtgaaagagatggatgtgcatacaggaagcatggacctgttggtcgacgatctgtttgcggaccgggacggggtttcgccaccgccccccaggccagccgggggtcccaagaacaccccagccgcccctccgctgtacgccaccggtcggctgagtcaggcccagctgatgccctcgccgcccatgcccgtcccccccgcggccctgtttaaccgtctcctcgacgatctgggcttcagcgcgggtcccgcgctgtgtaccatgctagatacctggaacgaggacctgttctctgggttcccgaccaacgccgacatgtaccgggagtgcaagtttctgtcgacgctgcccagcgacgtgatcgactggggggatgcgcacgtccccgagcgctccccgatcgacattcgcgcccacggcgacgtggcgttccccaccctgcccgccacccgcgacgagctgccttcgtactacgaggccatggcgcagtttttccgcggtgagctgcgggcgcgggaggagagctaccggaccgtgttggcaaatttttgctcggccctgtaccggtacctgcgcgccagcgttcggcagctacaccgccaggcacacatgcggggccgcaaccgcgacctgcgggagatgctgcgcaccacgatcgcggacaggtactaccgcgagaccgcgcgcctggcgcgcgtcctgtttctgcatctatacctctttctgagccgcgagatcctatgggccgcgtacgccgagcagatgatgcggcccgatctgttcgacggcctctgctgcgacctggagagctggcgccagttggcgtgtctgtttcagcccctgatgtttatcaacggatcgctcaccgtgcggggagttcccgtggaggcccggcgactgcgggagctaaaccacattcgcgagcacctgaacctcccgctggtgcgaagtgcggcggcggaggaacccggggcgcccctcacgaccccgcccgtcctgcagggcaaccaggcccgctcctctgggtactttatgctgctgatccgggccaagttggactcgtactccagcgtcgcgacctcggagggcgagtccgtcatgcgggagcacgcgtatagccgcgggcggaccagaaacaattacggatcgacaatcgagggcctgctcgacctcccggacgacgatgacgctcctgcggaggccgggctggtggcgccgcgcatgtcgtttctctccgcgggacaacgcccccgcagactgtccaccaccgcccccattaccgacgtcagcctgggagacgaactccgcctggacggcgaggaggtggatatgacgcccgccgacgccctggacgacttcgacttggagatgctgggggacgtggagtccccctccccgggaatgacccacgaccccgtctcgtatggggctttggacgtggacgattttgagtttgaacagatgtttaccgatgccatgggcattgacgactttggggggtag

SEQ ID NO: 2 - protein, 926 aa, Artificial Sequence
  Description of Artificial Sequence  Synthetic Peptide
MAELMPVGDNNFSDSEEGEGLEESADIKGEPHGLENMELRSLELSVVEPQPVFEASGAPDIYSSNKDLPPETPGAEDKGKSSKTKPFRCKPCQYEAESEEQFVHHIRVHSAKKFFVEESAEKQAKARESGSSTAEEGDFSKGPIRCDRCGYNTNRYDHYTAHLKHHTRAGDNERVYKCIICTYTTVSEYHWRKHLRNHFPRKVYTCGKCNYFSDRKNNYVQHVRTHTGERPYKCELCPYSSSQKTHLTRHMRTHSGEKPFKCDQCSYVASNQHEVTRHARQVHNGPKPLNCPHCDYKTADRSNFKKHVELHVNPRQFNCPVCDYAASKKCNLQYHFKSKHPTCPNKTMDVSKVKLKKTKKREADLPDNITNEKTEIEQTKIKGDVAGKKNEKSVKAEKRDVSKEKKPSNNVSVIQVTTRTRKSVTEVKEMDVHTGSMDLLVDDLFADRDGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDAMGIDDFGG

SEQ ID NO: 3 - DNA, 1694 bp, Artificial Sequence
  CDS (1)..(1692)
  Description of Artificial Sequence  Synthetic Primer
atggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttgaggcatcaggtgctccagatatttacagttcaaataaagatcttccccctgaaacacctggagcggaggacaaaggcaagagctcgaagaccaaaccctttcgctgtaagccatgccaatatgaagcagaatctgaagaacagtttgtgcatcacatcagagttcacagtgctaagaaattttttgtggaagagagtgcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacaggagaacgcccatataaatgtgaactttgtccttactcaagttctcagaagactcatctaactagacatatgcgtactcattcaggtgagaagccatttaaatgtgatcagtgcagttatgtggcctctaatcaacatgaagtaacccgccatgcaagacaggttcacaatgggcctaaacctcttaattgcccacactgtgattacaaaacagcagatagaagcaacttcaaaaaacatgtagagctacatgtgaacccacggcagttcaattgccctgtatgtgactatgcagcttccaagaagtgtaatctacagtatcacttcaaatctaagcatcctacttgtcctaataaaacaatggatgtctcaaaagtgaaactaaagaaaaccaaaaaacgagaggctgacttgcctgataatattaccaatgaaaaaacagaaatagaacaaacaaaaataaaaggggatgtggctggaaagaaaaatgaaaagtccgtcaaagcagagaaaagagatgtctcaaaagagaaaaagccttctaataatgtgtcagtgatccaggtgactaccagaactcgaaaatcagtaacagaggtgaaagagatggatgtgcatacaggaagcgaattcagcagaggcagaaccagaaacaactacggctctaccatcgagggcttgctcgacctcccagacgacgacgacgctccagccgaagcaggtctcgttgctccaagaatgtctttcctctccgctggacagagaccaagaagactttctaccaccgctccaatcaccgacgtttctttggttgacgaattgagattggacggcgaggaggttgacatgaccccagccgacgccttggacgacttcgacttggagatgttgggtgacgttgagtccccatccccaggcatgacccacgacccagtttcctacggcgctttggacgttgacgacttcgagtttgagcagatgttcaccgacgccttgggcatcgacgactttag

SEQ ID NO: 4 - protein, 564 aa, Artificial Sequence
  Description of Artificial Sequence  Synthetic Peptide
MAELMPVGDNNFSDSEEGEGLEESADIKGEPHGLENMELRSLELSVVEPQPVFEASGAPDIYSSNKDLPPETPGAEDKGKSSKTKPFRCKPCQYEAESEEQFVHHIRVHSAKKFFVEESAEKQAKARESGSSTAEEGDFSKGPIRCDRCGYNTNRYDHYTAHLKHHTRAGDNERVYKCIICTYTTVSEYHWRKHLRNHFPRKVYTCGKCNYFSDRKNNYVQHVRTHTGERPYKCELCPYSSSQKTHLTRHMRTHSGEKPFKCDQCSYVASNQHEVTRHARQVHNGPKPLNCPHCDYKTADRSNFKKHVELHVNPRQFNCPVCDYAASKKCNLQYHFKSKHPTCPNKTMDVSKVKLKKTKKREADLPDNITNEKTEIEQTKIKGDVAGKKNEKSVKAEKRDVSKEKKPSNNVSVIQVTTRTRKSVTEVKEMDVHTGSEFSRGRTRNNYGSTIEGLLDLPDDDDAPAEAGLVAPRMSFLSAGQRPRRLSTTAPITDVSLVDELRLDGEEVDMTPADALDDFDLEMLGDVESPSPGMTHDPVSYGALDVDDFEFEQMFTDALGIDDF

SEQ ID NO: 5 - DNA, 23 bp, Artificial Sequence
  Description of Artificial Sequence  Synthetic Primer
gtgcaaagccatttcagcaccac

SEQ ID NO: 6 - DNA, 20 bp, Mus musculus
ccggaarrcttttccagtga

SEQ ID NO: 7 - DNA, 20 bp, Mus musculus
aaacacaaagctggggacac

SEQ ID NO: 8 - DNA, 20 bp, Mus musculus
tccaaccgaaaaatgaggtc

SEQ ID NO: 9 - DNA, 20 bp, Mus musculus
ggcaggaagcagattacgag

SEQ ID NO: 10 - DNA, 20 bp, Mus musculus
ttgttttgtgccgtttacca

SEQ ID NO: 11 - DNA, 20 bp, Mus musculus
ggtaagagagccagccactg

SEQ ID NO: 12 - DNA, 20 bp, Mus musculus
tgtcgggtaactggaagacc

SEQ ID NO: 13 - DNA, 20 bp, Mus musculus
agttccacgatgagctgctt

SEQ ID NO: 14 - DNA, 26 bp, Mus musculus
cgctcgttgccaatagtgatgacctg

SEQ ID NO: 15 - DNA, 20 bp, Mus musculus
gtccacacccgccaccagtt